(19) United States
(12) Patent Application Publication
(10) Pub. No.: US 2002/0164687 A1
(43) Pub. Date: Nov. 7, 2002

(54) Platelet-derived growth factor C, DNA coding therefor, and uses thereof

(76) Inventors: Ulf Eriksson, Stockholm (SE); Karin Aase, Stockholm (SE); Xuri Li, Stockholm (SE); Annica Ponten, Stockholm (SE); Marko Uutela, Helsinki (FI); Kari Alitalo, Helsinki (FI); Arne Oestman, Uppsala (SE); Carl-Henrik Heldin, Uppsala (SE); Christer Betsholtz, Goeteborg (SE)

Correspondence Address: CROWELL & MORING LLP, INTELLECTUAL PROPERTY GROUP, P.O. BOX 14300, WASHINGTON, DC, 20044-4300, US

(21) Appl. No.: 09/852,209

(22) Filed: May 10, 2001

Related U.S. Application Data
(63) Continuation-in-part of application No. 09/410,349, filed on Sep. 30, 1999.
(60) Provisional application No. 60/102,461, filed on Sep. 30, 1998.
(60) Provisional application No. 60/108,109, filed on Nov. 12, 1998.
(60) Provisional application No. 60/110,749, filed on Dec. 3, 1998.
(60) Provisional application No. 60/113,002, filed on Dec. 18, 1998.
(60) Provisional application No. 60/135,426, filed on May 21, 1999.
(60) Provisional application No. 60/144,022, filed on Jul. 15, 1999.

(51) Int. Cl. C07K 14/475; C07H 21/04; C12P 21/02; C12N 5/06

(52) U.S. Cl. 435/69.1; 435/320.1; 435/325; 530/350; 536/23.5

(57) ABSTRACT

PDGF-C, a new member of the PDGF/VEGF family of growth factors, is described, as well as the nucleotide sequence encoding it, methods for producing it, antibodies and other antagonists to it, transfected and transformed host cells expressing it, pharmaceutical compositions containing it, and uses thereof in medical and diagnostic applications.

### CROSS REFERENCE TO RELATED APPLICATIONS

This application is a continuation-in-part of copending application Ser. No. 09/410,349, filed Sep. 30, 1999, which in turn claims the benefit of U.S. Provisional Application No. 60/102,461, filed Sep. 30, 1998; U.S. Provisional Application No. 60/108,109, filed Nov. 12, 1998; U.S. Provisional Application No. 60/110,749, filed Dec. 3, 1998; U.S. Provisional Application No. 60/113,002, filed Dec. 18, 1998; U.S. Provisional Application No. 60/135,426, filed May 21, 1999; and U.S. Provisional Application No. 60/144,022, filed Jul. 15, 1999.

### FIELD OF THE INVENTION

This invention relates to growth factors for connective tissue cells, fibroblasts, myofibroblasts and glial cells and/or to growth factors for endothelial cells, and in particular to a novel platelet-derived growth factor/vascular endothelial growth factor-like growth factor, a polynucleotide sequence encoding the factor, and to pharmaceutical and diagnostic compositions and methods utilizing or derived from the factor.

### BACKGROUND OF THE INVENTION

In the developing embryo, the primary vascular network is established by in situ differentiation of mesodermal cells in a process called vasculogenesis. It is believed that all subsequent processes involving the generation of new vessels in the embryo and neovascularization in adults, are governed by the sprouting or splitting of new capillaries from the pre-existing vasculature in a process called angiogenesis (Pepper et al., Enzyme & Protein, 1996 49 138-162; Breier et al., Dev. Dyn. 1995 204 228-239; Risau, Nature, 1997 386 671-674). Angiogenesis is not only involved in embryonic development and normal tissue growth, repair, and regeneration, but is also involved in the female reproductive cycle, establishment and maintenance of pregnancy, and in repair of wounds and fractures. In addition to angiogenesis which takes place in the normal individual, angiogenic events are involved in a number of pathological processes, notably tumor growth and metastasis, and other conditions in which blood vessel proliferation, especially of the microvascular system, is increased, such as diabetic retinopathy, psoriasis and arthropathies. Inhibition of angiogenesis is useful in preventing or alleviating these pathological processes.

On the other hand, promotion of angiogenesis is desirable in situations where vascularization is to be established or extended, for example after tissue or organ transplantation, or to stimulate establishment of collateral circulation in tissue infarction or arterial stenosis, such as in coronary heart disease and thromboangitis obliterans.

The angiogenic process is highly complex and involves the maintenance of the endothelial cells in the cell cycle, degradation of the extracellular matrix, migration and invasion of the surrounding tissue and finally, tube formation. The molecular mechanisms underlying the complex angiogenic processes are far from being understood.

Because of the crucial role of angiogenesis in so many physiological and pathological processes, factors involved in the control of angiogenesis have been intensively investigated. A number of growth factors have been shown to be involved in the regulation of angiogenesis; these include fibroblast growth factors (FGFs), platelet-derived growth factor (PDGF), transforming growth factor alpha (TGFα), and hepatocyte growth factor (HGF). See for example Folkman et al., J. Biol. Chem., 1992 267 10931-10934 for a review.

It has been suggested that a particular family of endothelial cell-specific growth factors, the vascular endothelial growth factors (VEGFs), and their corresponding receptors is primarily responsible for stimulation of endothelial cell growth and differentiation, and for certain functions of the differentiated cells. These factors are members of the PDGF family, and appear to act primarily via endothelial receptor tyrosine kinases (RTKs).

Nine different proteins have been identified in the PDGF family, namely two PDGFs (A and B), VEGF and six members that are closely related to VEGF. The six members closely related to VEGF are: VEGF-B, described in International Patent Application PCT/US96/02957 (WO 96/26736) and in U.S. Pat. Nos. 5,840,693 and 5,607,918 by Ludwig Institute for Cancer Research and The University of Helsinki; VEGF-C, described in Joukov et al., EMBO J., 1996 15 290-298 and Lee et al., Proc. Natl. Acad. Sci. USA, 1996 93 1988-1992; VEGF-D, described in International Patent Application No. PCT/US97/14696 (WO 98/07832), and Achen et al., Proc. Natl. Acad. Sci. USA, 1998 95 548-553; the placenta growth factor (PlGF), described in Maglione et al., Proc. Natl. Acad. Sci. USA, 1991 88 9267-9271; VEGF2, described in International Patent Application No. PCT/US94/05291 (WO 95/24473) by Human Genome Sciences, Inc; and VEGF3, described in International Patent Application No. PCT/US95/07283 (WO 96/39421) by Human Genome Sciences, Inc. Each VEGF family member has between 30% and 45% amino acid sequence identity with VEGF. The VEGF family members share a VEGF homology domain which contains the six cysteine residues which form the cysteine knot motif. Functional characteristics of the VEGF family include varying degrees of mitogenicity for endothelial cells, induction of vascular permeability and angiogenic and lymphangiogenic properties.

Vascular endothelial growth factor (VEGF) is a homodimeric glycoprotein that has been isolated from several sources. VEGF shows highly specific mitogenic activity for endothelial cells. VEGF has important regulatory functions in the formation of new blood vessels during embryonic vasculogenesis and in angiogenesis during adult life (Carmeliet et al., Nature, 1996 380 435-439; Ferrara et al., Nature, 1996 380 439-442; reviewed in Ferrara and Davis-Smyth, Endocrine Rev., 1997 18 4-25). The significance of the role played by VEGF has been demonstrated in studies showing that inactivation of a single VEGF allele results in embryonic lethality due to failed development of the vasculature (Carmeliet et al., Nature, 1996 380 435-439; Ferrara et al., Nature, 1996 380 439-442). In addition VEGF has strong chemoattractant activity towards monocytes, can induce the plasminogen activator and the plasminogen activator inhibitor in endothelial cells, and can also induce microvascular permeability. Because of the latter activity, it is sometimes referred to as vascular permeability factor (VPF). The isolation and properties of VEGF have been reviewed; see Ferrara et al., J. Cellular Biochem., 1991 47 211-218 and Connolly, J. Cellular Biochem., 1991 47 219-223. Alterative mRNA splicing of a single VEGF gene gives rise to five isoforms of VEGF.

VEGF-B has similar angiogenic and other properties to those of VEGF, but is distributed and expressed in tissues differently from VEGF. In particular, VEGF-B is very strongly expressed in heart, and only weakly in lung, whereas the reverse is the case for VEGF. This suggests that VEGF and VEGF-B, despite the fact that they are co-expressed in many tissues, may have functional differences.

VEGF-B was isolated using a yeast co-hybrid interaction trap screening technique by screening for cellular proteins which might interact with cellular resinoid acid-binding protein type I (CRABP-I). Its isolation and characteristics are described in detail in PCT/US96/02957 and in Olofsson et al., Proc. Natl. Acad. Sci. USA, 1996 93 2576-2581.

VEGF-C was isolated from conditioned media of the PC-3 prostate adenocarcinoma cell line (CRL1435) by screening for ability of the medium to produce tyrosine phosphorylation of the endothelial cell-specific receptor tyrosine kinase VEGFR-3 (Flt4), using cells transfected to express VEGFR-3. VEGF-C was purified using affinity chromatography with recombinant VEGFR-3, and was cloned from a PC-3 cDNA library. Its isolation and characteristics are described in detail in Joukov et al., EMBO J., 1996 15 290-298.

VEGF-D was isolated from a human breast cDNA library, commercially available from Clontech, by screening with an expressed sequence tag obtained from a human cDNA library designated “Soares Breast 3NbHBst” as a hybridization probe (Achen et al., Proc. Natl. Acad. Sci. USA, 1998 95 548-553). Its isolation and characteristics are described in detail in International Patent Application No. PCT/US97/14696 (WO98/07832).

The VEGF-D gene is broadly expressed in the adult human, but is certainly not ubiquitously expressed. VEGF-D is strongly expressed in heart, lung and skeletal muscle. Intermediate levels of VEGF-D are expressed in spleen, ovary, small intestine and colon, and a lower expression occurs in kidney, pancreas, thymus, prostate and testis. No VEGF-D mRNA was detected in RNA from brain, placenta, liver or peripheral blood leukocytes.

PlGF was isolated from a term placenta cDNA library. Its isolation and characteristics are described in detail in Maglione et al., Proc. Natl. Acad. Sci. USA, 1991 88 9267-9271. Presently its biological function is not well understood. 34

VEGF2 was isolated from a highly tumorgenic, oestrogen-independent human breast cancer cell line. While this molecule is stated to have about 22% homology to PDGF and 30% homology to VEGF, the method of isolation of the gene encoding VEGF2 is unclear, and no characterization of the biological activity is disclosed.

VEGF3 was isolated from a cDNA library derived from colon tissue. VEGF3 is stated to have about 36% identity and 66% similarity to VEGF. The method of isolation of the gene encoding VEGF3 is unclear and no characterization of the biological activity is disclosed.

Similarity between two proteins is determined by comparing the amino acid sequence and conserved amino acid substitutions of one of the proteins to the sequence of the second protein, whereas identity is determined without including the conserved amino acid substitutions.

PDGF/VEGF family members act primarily by binding to receptor tyrosine kinases. Five endothelial cell-specific receptor tyrosine kinases have been identified, namely VEGFR-1 (Flt-1), VEGFR-2 (KDR/Flk-1), VEGFR-3 (Flt4), Tie and Tek/Tie-2. All of these have the intrinsic tyrosine kinase activity which is necessary for signal transduction. The essential, specific role in vasculogenesis and angiogenesis of VEGFR-1, VEGFR-2, VEGFR-3, Tie and Tek/Tie-2 has been demonstrated by targeted mutations inactivating these receptors in mouse embryos.

The only receptor tyrosine kinases known to bind VEGFs are VEGFR-1, VEGFR-2 and VEGFR-3. VEGFR-1 and VEGFR-2 bind VEGF with high affinity, and VEGFR-1 also binds VEGF-B and PlGF. VEGF-C has been shown to be the ligand for VEGFR-3, and it also activates VEGFR-2 (Joukov et al., The EMBO Journal, 1996 15 290-298). VEGF-D binds to both VEGFR-2 and VEGFR-3. A ligand for Tek/Tie-2 has been described in International Patent Application No. PCT/US95/12935 (WO 96/11269) by Regeneron Pharmaceuticals, Inc. The ligand for Tie has not yet been identified.

Recently, a novel 130-135 kDa VEGF isoform specific receptor has been purified and cloned (Soker et al., Cell, 1998 92 735-745). The VEGF receptor was found to specifically bind the VEGF₁₆₅ isoform via the exon 7 encoded sequence, which shows weak affinity for heparin (Soker et al., Cell, 1998 92 735-745). Surprisingly, the receptor was shown to be identical to human neuropilin-1 (NP1), a receptor involved in early stage neuromorphogenesis. PlGF-2 also appears to interact with NP-1 (Migdal et al., J. Biol. Chem., 1998 273 22272-22278).

VEGFR-1, VEGFR-2 and VEGFR-3 are expressed differently by endothelial cells. Both VEGFR-1 and VEGFR-2 are expressed in blood vessel endothelia (Oelrichs et al., Oncogene, 1992 8 11-18; Kaipainen et al., J. Exp. Med., 1993 178 2077-2088; Dumont et al., Dev. Dyn., 1995 203 80-92; Fong et al., Dev. Dyn., 1996 207 1-10) and VEGFR-3 is mostly expressed in the lymphatic endothelium of adult tissues (Kaipainen et al., Proc. Natl. Acad. Sci. USA, 1995 9 3566-3570). VEGFR-3 is also expressed in the blood vasculature surrounding tumors.

Disruption of the VEGFR genes results in aberrant development of the vasculature leading to embryonic lethality around midgestation. Analysis of embryos carrying a completely inactivated VEGFR-1 gene suggests that this receptor is required for functional organization of the endothelium (Fong et al., Nature, 1995 376 66-70). However, deletion of the intracellular tyrosine kinase domain of VEGFR-1 generates viable mice with a normal vasculature (Hiratsuka et al., Proc. Natl. Acad. Sci. USA 1998 95 9349-9354). The reasons underlying these differences remain to be explained but suggest that receptor signalling via the tyrosine kinase is not required for the proper function of VEGFR-1. Analysis of homozygous mice with inactivated alleles of VEGFR-2 suggests that this receptor is required for endothelial cell proliferation, hematopoesis and vasculogenesis (Shalaby et al., Nature, 1995 376 62-66; Shalaby et al., Cell, 1997 89 981-990). Inactivation of VEGFR-3 results in cardiovascular failure due to abnormal organization of the large vessels (Dumont et al. Science, 1998 282 946-949).

Although VEGFR-1 is mainly expressed in endothelial cells during development, it can also be found in hematopoetic precursor cells during early stages of embryogenesis (Fong et al., Nature, 1995 376 66-70). In adults, monocytes and macrophages also express this receptor (Barleon et al., Blood, 1996 87 3336-3343). In embryos, VEGFR-1 is expressed by most, if not all, vessels (Breier et al., Dev. Dyn., 1995 204 228-239; Fong et al., Dev. Dyn., 1996 207 1-10).

The receptor VEGFR-3 is widely expressed on endothelial cells during early embryonic development but as embryogenesis proceeds becomes restricted to venous endothelium and then to the lymphatic endothelium (Kaipainen et al., Cancer Res., 1994 54 6571-6577; Kaipainen et al., Proc. Natl. Acad. Sci. USA, 1995 92 3566-3570). VEGFR-3 is expressed on lymphatic endothelial cells in adult tissues. This receptor is essential for vascular development during embryogenesis. Targeted inactivation of both copies of the VEGFR-3 gene in mice resulted in defective blood vessel formation characterized by abnormally organized large vessels with defective lumens, leading to fluid accumulation in the pericardial cavity and cardiovascular failure at post-coital day 9.5. On the basis of these findings it has been proposed that VEGFR-3 is required for the maturation of primary vascular networks into larger blood vessels. However, the role of VEGFR-3 in the development of the lymphatic vasculature could not be studied in these mice because the embryos died before the lymphatic system emerged. Nevertheless it is assumed that VEGFR-3 plays a role in development of the lymphatic vasculature and lymphangiogenesis given its specific expression in lymphatic endothelial cells during embryogenesis and adult life. This is supported by the finding that ectopic expression of VEGF-C, a ligand for VEGFR-3, in the skin of transgenic mice, resulted in lymphatic endothelial cell proliferation and vessel enlargement in the dermis. Furthermore this suggests that VEGF-C may have a primary function in lymphatic endothelium, and a secondary function in angiogenesis and permeability regulation which is shared with VEGF (Joukov et al., EMBO J., 1996 15 290-298).

Some inhibitors of the VEGF/VEGF-receptor system have been shown to prevent tumor growth via an anti-angiogenic mechanism; see Kim et al., Nature, 1993 362 841-844 and Saleh et al., Cancer Res., 1996 56 393-401.

As mentioned above, the VEGF family of growth factors are members of the PDGF family. PDGF plays a important role in the growth and/or motility of connective tissue cells, fibroblasts, myofibroblasts and glial cells (Heldin et al., “Structure of platelet-derived growth factor: Implications for functional properties”, Growth Factor, 1993 8 245-252). In adults, PDGF stimulates wound healing (Robson et al., Lancet, 1992 339 23-25). Structurally, PDGF isoforms are disulfide-bonded dimers of homologous A- and B-polypeptide chains, arranged as homodimers (PDGF-AA and PDGF-BB) or a heterodimer (PDGF-AB).

PDGF isoforms exert their effects on target cells by binding to two structurally related receptor tyrosine kinases (RTKs). The alpha-receptor binds both the A- and B-chains of PDGF, whereas the beta-receptor binds only the B-chain. These two receptors are expressed by many in vitro grown cell lines, and are mainly expressed by mesenchymal cells in vivo. The PDGFs regulate cell proliferation, cell survival and chemotaxis of many cell types in vitro (reviewed in Heldin et al., Biochim Biophys Acta., 1998 1378 F79-113). In vivo, they exert their effects in a paracrine mode since they often are expressed in epithelial (PDGF-A) or endothelial cells (PDGF-B) in close apposition to the PDGFR expressing mesenchyme. In tumor cells and in cell lines grown in vitro, coexpression of the PDGFs and the receptors generate autocrine loops which are important for cellular transformation (Betsholtz et al., Cell, 1984 39 447-57; Keating et al., J. R. Coll Surg Edinb., 1990 35 172-4). Overexpression of the PDGFs have been observed in several pathological conditions, including maligancies, arteriosclerosis, and fibroproliferative diseases (reviewed in Heldin et al., The Molecular and Cellular Biology of Wound Repair, New York: Plenum Press, 1996, 249-273).

The importance of the PDGFs as regulators of cell proliferation and survival are well illustrated by recent gene targeting studies in mice that have shown distinct physiological roles for the PDGFs and their receptors despite the overlapping ligand specificities of the PDGFRs. Homozygous null mutations for either of the two PDGF ligands or the receptors are lethal. Approximately 50% of the homozygous PDGF-A deficient mice have an early lethal phenotype, while the surviving animals have a complex postnatal phenotype with lung emphysema due to improper alveolar septum formation because of a lack of alveolar myofibroblasts (Boström et al., Cell, 1996 85 863-873). The PDGF-A deficient mice also have a dermal phenotype characterized by thin dermis, misshapen hair follicles and thin hair (Karlsson et al., Development, 1999 126 2611-2). PDGF-A is also required for normal development of oligodendrocytes and subsequent myelination of the central nervous system (Fruttiger et al., Development, 1999 126 457-67). The phenotype of PDGFR-alpha deficient mice is more severe with early embryonic death at E10, incomplete cephalic closure, impaired neural crest development, cardiovascular defects, skeletal defects, and odemas [Soriano et al., Development, 1997 124 2691-70). The PDGF-B and PDGFR-beta deficient mice develop similar phenotypes that are characterized by renal, hematological and cardiovascular abnormalities (Leveen et al., Genes Dev., 1994 8 1875-1887; Soriano et al., Genes Dev., 1994 8 1888-96; Lindahl et al., Science, 1997 277 242-5; Lindahl, Development, 1998 125 3313-2), where the renal and cardiovascular defects, at least in part, are due to the lack of proper recruitment of mural cells (vascular smooth muscle cells, pericytes or mesangial cells) to blood vessels (Leveen et al., Genes Dev., 1994 8 1875-1887; Lindahl et al., Science, 1997 277 242-5; Lindahl et al., Development, 1998 125 3313-2).

Administration of growth factors such as VEGF and FGF-2 has been considered a possible approach for the therapeutic treatment of ischemic heart and limb disorders. However, both animal studies and early clinical trials with VEGF angiogenesis have encountered severe problems (Carmeliet, Nat Med 2000 6 1102-3; Yancopoulos et al., Nature 2000 407 242-8; Veikkola et al., Semin Cancer Biol 1999 9 211-20; Dvorak et al., Semin Perinatol 2000 24 75-8; Lee et al., Circulation 2000 102 898-901). VEGF-stimulated microvessels are disorganized, sinusoidal and dilated, much like those found in tumors. Moreover, these vessels are usually leaky, poorly perfused, torturous and likely to rupture and regress. Thus, these vessels have limited ability to improve the ischemic conditions of myocardium. In addition, the leakage of blood vessels induced by VEGF (also known as Vascular Permeability Factor) could cause cardiac edema that leads to heart failure. Unregulated VEGF expression in the myocardium also could lead to the development of hemangioma or the growth of micrometastases in distal organs instead of functional vessels. Thus, despite the efforts of the prior art, there remains a substantial need for new angiogenic factors and new methods of angiogenic therapy.

### SUMMARY OF THE INVENTION

The invention generally provides an isolated novel growth factor which has the ability to stimulate and/or enhance proliferation or differentiation and/or growth and/or motility of cells expressing a PDGF-C receptor including, but not limited to, endothelial cells, connective tissue cells, myofibroblasts and glial cells, an isolated polynucleotide sequence encoding the novel growth factor, and compositions useful for diagnostic and/or therapeutic applications.

According to one aspect, the invention provides an isolated and purified nucleic acid molecule which comprises a polynucleotide sequence having at least 85% identity, more preferably at least 90%, and most preferably at least 95% identity to at least nucleotides 37-1071 of the sequence set out in FIG. 1 (SEQ ID NO:2), at least nucleotides 6-956 of the sequence set out in FIG. 3 (SEQ ID NO:3) or at least nucleotides 196 to 1233 of the sequence set out in FIG. 5 (SEQ ID NO:6). The sequence of at least nucleotides 37-1071 of the sequence set out in FIG. 1 (SEQ ID NO:2) or at least nucleotides 196 to 1233 of the sequence set out in FIG. 5 (SEQ ID NO:6) encodes a novel polypeptide, designated PDGF-C (formally designated “VEGF-F”), which is structurally homologous to PDGF-A, PDGF-B, VEGF, VEGF-B, VEGF-C and VEGF-D. In a preferred embodiment, the nucleic acid molecule is a cDNA which comprises at least nucleotides 37-1071 of the sequence set out in FIG. 1 (SEQ ID NO:2), at least nucleotides 6-956 of the sequence set out in FIG. 3 (SEQ ID NO:3) or at least nucleotides 196 to 1233 of the sequence set out in FIG. 5 (SEQ ID NO:6). This aspect of the invention also encompasses DNA molecules having a sequence such that they hybridize under stringent conditions with at least nucleotides 37-1071 of the sequence set out in FIG. 1 (SEQ ID NO:2), at least nucleotides 6-956 of the sequence set out in FIG. 3 (SEQ ID NO:3) or at least nucleotides 196 to 1233 of the sequence set out in FIG. 5 (SEQ ID NO:6) or fragments thereof.

According to a second aspect, the polypeptide of the invention has the ability to stimulate and/or enhance proliferation and/or differentiation and/or growth and/or motility of cells expressing a PDGF-C receptor including, but not limited to, endothelial cells, connective tissue cells, myofibroblasts and glial cells and comprises a sequence of amino acids corresponding to the amino acid sequence set out in FIG. 2 (SEQ ID NO:3), FIG. 4 (SEQ ID NO:5) or FIG. 6 (SEQ ID NO: 7), or a fragment or analog thereof which has the ability to stimulate and/or enhance proliferation and/or differentiation and/or growth and/or motility of cells expressing a PDGF-C receptor including, but not limited to, endothelial cells, connective tissue cells (such as fibroblasts), myofibroblasts and glial cells. Preferably the polypeptides have at least 85% identity, more preferably at least 90%, and most preferably at least 95% identity to the amino acid sequence of in FIG. 2 (SEQ ID NO:3), FIG. 4 (SEQ ID NO:5) or FIG. 6 (SEQ ID NO:7), or a fragment or analog thereof having the biological activity of PDGF-C. A preferred fragment is a truncated form of PDGF-C comprising a portion of the PDGF/VEGF homology domain (PVHD) of PDGF-C. The minimal domain is residues 230-345. However, the domain can extend towards the N terminus up to residue 164. Herein the PVHD is defined as truncated PDGF-C. The truncated PDGF-C is an activated form of PDGF-C.

As used in this application, percent sequence identity is determined by using the alignment tool of “MEGALIGN” from the Lasergene package (DNASTAR, Ltd. Abacus House, Manor Road, West Ealing, London W130AS United Kingdom) and using its preset conditions. The alignment is then refined manually, and the number of identities are estimated in the regions available for a comparison.

Preferably the polypeptide or the encoded polypeptide from a polynucleotide has the ability to stimulate one or more of proliferation, differentiation, motility, survival or vascular permeability of cells expressing a PDGF-C receptor including, but not limited to, vascular endothelial cells, lymphatic endothelial cells, connective tissue cells (such as fibroblasts), myofibroblasts and glial cells. Preferably the polypeptide or the encoded polypeptide from a polynucleotide has the ability to stimulate wound healing. PDGF-C can also have antagonistic effects on cells, but are included in the biological activities of PDGF-C. These abilities are referred to hereinafter as “biological activities of PDGF-C” and can be readily tested by methods known in the art.

As used herein, the term “PDGF-C” collectively refers to the polypeptides of FIG. 2 (SEQ ID NO:3), FIG. 4 (SEQ ID NO:5) or FIG. 6 (SEQ ID NO:7), and fragments or analogs thereof which have the biological activity of PDGF-C as defined above, and to a polynucleotide which can code for PDGF-C, or a fragment or analog thereof having the biological activity of PDGF-C. The polynucleotide can be naked and/or in a vector or liposome.

In another preferred aspect, the invention provides a polypeptide possessing an amino acid sequence:

PXCLLVXRCGGXCXCC (SEQ ID NO:1)

which is unique to PDGF-C and differs from the other members of the PDGF/VEGF family of growth factors because of the insertion of the three amino acid residues (NCA) between the third and fourth cysteines (see FIG. 9—SEQ ID NOs:8-17).

Polypeptides comprising conservative substitutions, insertions, or deletions, but which still retain the biological activity of PDGF-C are clearly to be understood to be within the scope of the invention. Persons skilled in the art will be well aware of methods which can readily be used to generate such polypeptides, for example the use of site-directed mutagenesis, or specific enzymatic cleavage and ligation. The skilled person will also be aware that peptidomimetic compounds or compounds in which one or more amino acid residues are replaced by a non-naturally occurring amino acid or an amino acid analog may retain the required aspects of the biological activity of PDGF-C. Such compounds can readily be made and tested by methods known in the art, and are also within the scope of the invention.

In addition, possible variant forms of the PDGF-C polypeptide which may result from alternative splicing, as are known to occur with VEGF and VEGF-B, and naturally-occurring allelic variants of the nucleic acid sequence encoding PDGF-C are encompassed within the scope of the invention. Allelic variants are well known in the art, and represent alternative forms or a nucleic acid sequence which comprise substitution, deletion or addition of one or more nucleotides, but which do not result in any substantial functional alteration of the encoded polypeptide.

Such variant forms of PDGF-C can be prepared by targeting non-essential regions of the PDGF-C polypeptide for modification. These non-essential regions are expected to fall outside the strongly-conserved regions indicated in FIG. 9 (SEQ ID NOs:8-17). In particular, the growth factors of the PDGF family, including VEGF, are dimeric, and VEGF, VEGF-B, VEGF-C, VEGF-D, PlGF, PDGF-A and PDGF-B show complete conservation of eight cysteine residues in the N-terminal domains, i.e. the PDGF/VEGF-like domains (Olofsson et al., Proc. Natl. Acad. Sci. USA, 1996 93 2576-2581; Joukov et al., EMBO J., 1996 15 290-298). These cysteines are thought to be involved in intra- and inter-molecular disulfide bonding. In addition there are further strongly, but not completely, conserved cysteine residues in the C-terminal domains. Loops 1, 2 and 3 of each subunit, which are formed by intra-molecular disulfide bonding, are involved in binding to the receptors for the PDGF/VEGF family of growth factors (Andersson et al., Growth Factors, 1995 12 159-164).

Persons skilled in the art thus are well aware that these cysteine residues should be preserved in any proposed variant form, and that the active sites present in loops 1, 2 and 3 also should be preserved. However, other regions of the molecule can be expected to be of lesser importance for biological function, and therefore offer suitable targets for modification. Modified polypeptides can readily be tested for their ability to show the biological activity of PDGF-C by routine activity assay procedures such as the fibroblast proliferation assay of Example 6.

It is contemplated that some modified PDGF-C polypeptides will have the ability to bind to PDGF-C receptors on cells including, but not limited to, endothelial cells, connective tissue cells, myofibroblasts and/or glial cells, but will be unable to stimulate cell proliferation, differentiation, migration, motility or survival or to induce vascular proliferation, connective tissue development or wound healing. These modified polypeptides are expected to be able to act as competitive or non-competitive inhibitors of the PDGF-C polypeptides and growth factors of the PDGF/VEGF family, and to be useful in situations where prevention or reduction of the PDGF-C polypeptide or PDGF/VEGF family growth factor action is desirable. Thus such receptor-binding but non-mitogenic, non-differentiation inducing, non-migration inducing, non-motility inducing, non-survival promoting, non-connective tissue development promoting, non-wound healing or non-vascular proliferation inducing variants of the PDGF-C polypeptide are also within the scope of the invention, and are referred to herein as “receptor-binding but otherwise inactive variant”. Because PDGF-C forms a dimer in order to activate its only known receptor, it is contemplated that one monomer comprises the receptor-binding but otherwise inactive variant modified PDGF-C polypeptide and a second monomer comprises a wild-type PDGF-C or a wild-type growth factor of the PDGF/VEGF family. These dimers can bind to its corresponding receptor but cannot induce downstream signaling.

It is also contemplated that there are other modified PDGF-C polypeptides that can prevent binding of a wild-type PDGF-C or a wild-type growth factor of the PDGF/VEGF family to its corresponding receptor on cells including, but not limited to, endothelial cells, connective tissue cells (such as fibroblasts), myofibroblasts and/or glial cells. Thus these dimers will be unable to stimulate endothelial cell proliferation, differentiation, migration, survival, or induce vascular permeability, and/or stimulate proliferation and/or differentiation and/or motility of connective tissue cells, myofibroblasts or glial cells. These modified polypeptides are expected to be able to act as competitive or non-competitive inhibitors of the PDGF-C growth factor or a growth factor of the PDGF/VEGF family, and to be useful in situations where prevention or reduction of the PDGF-C growth factor or PDGF/VEGF family growth factor action is desirable. Such situations include the tissue remodeling that takes place during invasion of tumor cells into a normal cell population by primary or metastatic tumor formation. Thus such the PDGF-C or PDGF/VEGF family growth factor-binding but non-mitogenic, non-differentiation inducing, non-migration inducing, non-motility inducing, non-survival promoting, non-connective tissue promoting, non-wound healing or non-vascular proliferation inducing variants of the PDGF-C growth factor are also within the scope of the invention, and are referred to herein as “the PDGF-C growth factor-dimer forming but otherwise inactive or interfering variants”.

An example of a PDGF-C growth factor-dimer forming but otherwise inactive or interfering variant is where the PDGF-C has a mutation which prevents cleavage of CUB domain from the protein. It is further contemplated that a PDGF-C growth factor-dimer forming but otherwise inactive or interfering variant could be made to comprise a monomer, preferably an activated monomer, of VEGF, VEGF-B, VEGF-C, VEGF-D, PDGF-C, PDGF-A, PDGF-B or PlGF linked to a CUB domain that has a mutation which prevents cleavage of CUB domain from the protein. Dimers formed with the above mentioned PDGF-C growth factor-dimer forming but otherwise inactive or interfering variants and the monomers linked to the mutant CUB domain would be unable to bind to their corresponding receptors.

A variation on this contemplation would be to insert a proteolytic site between an activated monomer of VEGF, VEGF-B, VEGF-C, VEGF-D, PDGF-C, PDGF-A, PDGF-B or PlGF and the mutant CUB domain linkage which is dimerized to an activated monomer of VEGF, VEGF-B, VEGF-C, VEGF-D, PDGF-C, PDGF-A, PDGF-B or PlGF. An addition of the specific protease(s) for this proteolytic site would cleave the CUB domain and thereby release an activated dimer that can then bind to its corresponding receptor. In this way, a controlled release of an activated dimer is made possible.

According to a third aspect, the invention provides a purified and isolated nucleic acid encoding a polypeptide or polypeptide fragment of the invention as defined above. The nucleic acid may be DNA, genomic DNA, cDNA or RNA, and may be single-stranded or double stranded. The nucleic acid may be isolated from a cell or tissue source, or of recombinant or synthetic origin. Because of the degeneracy of the genetic code, the person skilled in the art will appreciate that many such coding sequences are possible, where each sequence encodes the amino acid sequence shown in FIG. 2 (SEQ ID NO:3), FIG. 4 (SEQ ID NO:5) or FIG. 6 (SEQ ID NO:7), a bioactive fragment or analog thereof, a receptor-binding but otherwise inactive or partially inactive variant thereof or a PDGF-C-dimer forming but otherwise inactive or interfering variants thereof.

A fourth aspect of the invention provides vectors comprising the cDNA of the invention or a nucleic acid molecule according to the third aspect of the invention, and host cells transformed or transfected with nucleic acids molecules or vectors of the invention. These may be eukaryotic or prokaryotic in origin. These cells are particularly suitable for expression of the polypeptide of the invention, and include insect cells such as Sf9 cells, obtainable from the American Type Culture Collection (ATCC SRL-171), transformed with a baculovirus vector, and the human embryo kidney cell line 293-EBNA transfected by a suitable expression plasmid. Preferred vectors of the invention are expression vectors in which a nucleic acid according to the invention is operatively connected to one or more appropriate promoters and/or other control sequences, such that appropriate host cells transformed or transfected with the vectors are capable of expressing the polypeptide of the invention. Other preferred vectors are those suitable for transfection of mammalian cells, or for gene therapy, such as adenoviral-, vaccinia- or retroviral-based vectors or liposomes. A variety of such vectors is known in the art.

The invention also provides a method of making a vector capable of expressing a polypeptide encoded by a nucleic acid according to the invention, comprising the steps of operatively connecting the nucleic acid to one or more appropriate promoters and/or other control sequences, as described above.

The invention further provides a method of making a polypeptide according to the invention, comprising the steps of expressing a nucleic acid or vector of the invention in a host cell, and isolating the polypeptide from the host cell or from the host cell's growth medium.

In yet a further aspect, the invention provides an antibody specifically reactive with a polypeptide of the invention or a fragment of the polypeptide. This aspect of the invention includes antibodies specific for the variant forms, immunoreactive fragments, analogs and recombinants of PDGF-C. Such antibodies are useful as inhibitors or agonists of PDGF-C and as diagnostic agents for detecting and quantifying PDGF-C. Polyclonal or monoclonal antibodies may be used. Monoclonal and polyclonal antibodies can be raised against polypeptides of the invention or fragment or analog thereof using standard methods in the art. In addition the polypeptide can be linked to an epitope tag, such as the FLAG® octapeptide (Sigma, St. Louis, Mo.), to assist in affinity purification. For some purposes, for example where a monoclonal antibody is to be used to inhibit effects of PDGF-C in a clinical situation, it may be desirable to use humanized or chimeric monoclonal antibodies. Such antibodies may be further modified by addition of cytotoxic or cytostatic drugs. Methods for producing these, including recombinant DNA methods, are also well known in the art.

This aspect of the invention also includes an antibody which recognizes PDGF-C and is suitably labeled.

Polypeptides or antibodies according to the invention may be labeled with a detectable label, and utilized for diagnostic purposes. Similarly, the thus-labeled polypeptide of the invention may be used to identify its corresponding receptor in situ. The polypeptide or antibody may be covalently or non-covalently coupled to a suitable supermagnetic, paramagnetic, electron dense, ecogenic or radioactive agent for imaging. For use in diagnostic assays, radioactive or non-radioactive labels may be used. Examples of radioactive labels include a radioactive atom or group, such as ¹²⁵I or ³²P. Examples of non-radioactive labels include enzymatic labels, such as horseradish peroxidase or fluorimetric labels, such as fluorescein-5-isothiocyanate (FITC). Labeling may be direct or indirect, covalent or non-covalent.

Clinical applications of the invention include diagnostic applications, acceleration of angiogenesis in tissue or organ transplantation, or stimulation of wound healing, or connective tissue development, or to establish collateral circulation in tissue infarction or arterial stenosis, such as coronary artery disease, and inhibition of angiogenesis in the treatment of cancer or of diabetic retinopathy and inhibition of tissue remodeling that takes place during invasion of tumor cells into a normal cell population by primary or metastatic tumor formation. Quantitation of PDGF-C in cancer biopsy specimens may be useful as an indicator of future metastatic risk.

PDGF-C may also be relevant to a variety of lung conditions. PDGF-C assays could be used in the diagnosis of various lung disorders. PDGF-C could also be used in the treatment of lung disorders to improve blood circulation in the lung and/or gaseous exchange between the lungs and the blood stream. Similarly, PDGF-C could be used to improve blood circulation to the heart and O₂ gas permeability in cases of cardiac insufficiency. In a like manner, PDGF-C could be used to improve blood flow and gaseous exchange in chronic obstructive airway diseases.

Thus the invention provides a method of stimulation of angiogenesis, lymphangiogenesis, neovascularization, connective tissue development and/or wound healing in a mammal in need of such treatment, comprising the step of administering an effective dose of PDGF-C, or a fragment or an analog thereof which has the biological activity of PDGF-C to the mammal. Optionally the PDGF-C, or fragment or analog thereof may be administered together with, or in conjunction with, one or more of VEGF, VEGF-B, VEGF-C, VEGF-D, PlGF, PDGF-A, PDGF-B, FGF and/or heparin.

Conversely, PDGF-C antagonists (e.g. antibodies and/or competitive or noncompetitive inhibitors of binding of PDGF-C in both dimer formation and receptor binding) could be used to treat conditions, such as congestive heart failure, involving accumulation of fluid in, for example, the lung resulting from increases in vascular permeability, by exerting an offsetting effect on vascular permeability in order to counteract the fluid accumulation. PDGF-C can also be used to treat fibrotic conditions including those found in the lung, kidney and liver. Administrations of PDGF-C could be used to treat malabsorptive syndromes in the intestinal tract, liver or kidneys as a result of its blood circulation increasing and vascular permeability increasing activities.

Thus, the invention provides a method of inhibiting angiogenesis, lymphangiogenesis, neovascularization, connective tissue development and/or wound healing in a mammal in need of such treatment, comprising the step of administering an effective amount of an antagonist of PDGF-C to the mammal. The antagonist may be any agent that prevents the action of PDGF-C, either by preventing the binding of PDGF-C to its corresponding receptor on the target cell, or by preventing activation of the receptor, such as using receptor-binding PDGF-C variants. Suitable antagonists include, but are not limited to, antibodies directed against PDGF-C; competitive or non-competitive inhibitors of binding of PDGF-C to the PDGF-C receptor(s), such as the receptor-binding or PDGF-C dimer-forming but non-mitogenic PDGF-C variants referred to above; compounds that bind to PDGF-C and/or modify or antagonize its function, and anti-sense nucleotide sequences as described below.

A method is provided for determining agents that bind to an activated truncated form of PDGF-C. The method comprises contacting an activated truncated form of PDGF-C with a test agent and monitoring binding by any suitable means. Agents can include both compounds and other proteins.

The invention provides a screening system for discovering agents that bind an activated truncated form of PDGF-C. The screening system comprises preparing an activated truncated form of PDGF-C, exposing the activated truncated form of PDGF-C to a test agent, and quantifying the binding of said agent to the activated truncated form of PDGF-C by any suitable means. This screening system can also be used to identify agents which inhibit the proteolytic cleavage of the full length PDGF-C protein and thereby prevent the release of the activated truncated form of PDGF-C. For this use, the full length PDGF-C must be prepared.

Use of this screen system provides a means to determine compounds that may alter the biological function of PDGF-C. This screening method may be adapted to large-scale, automated procedures such as a PANDEX® (Baxter-Dade Diagnostics) system, allowing for efficient high-volume screening of potential therapeutic agents.

For this screening system, an activated truncated form of PDGF-C or full length PDGF-C is prepared as described herein, preferably using recombinant DNA technology. A test agent, e.g. a compound or protein, is introduced into a reaction vessel containing the activated truncated form of or full length PDGF-C. Binding of the test agent to the activated truncated form of or full length PDGF-C is determined by any suitable means which include, but is not limited to, radioactively- or chemically-labeling the test agent. Binding of the activated truncated form of or full length PDGF-C may also be carried out by a method disclosed in U.S. Pat. No. 5,585,277, which is incorporated by reference. In this method, binding of the test agent to the activated truncated form of or full length PDGF-C is assessed by monitoring the ratio of folded protein to unfolded protein. Examples of this monitoring can include, but are not limited to, monitoring the sensitivity of the activated truncated form of or full length PDGF-C to a protease, or amenability to binding of the protein by a specific antibody against the folded state of the protein.

Those of skill in the art will recognize that IC₅₀ values are dependent on the selectivity of the agent tested. For example, an agent with an IC₅₀ which is less than 10 nM is generally considered an excellent candidate for drug therapy. However, an agent which has a lower affinity, but is selective for a particular target, may be an even better candidate. Those skilled in the art will recognize that any information regarding the binding potential, inhibitory activity or selectivity of a particular agent is useful toward the development of pharmaceutical products.

Where a PDGF-C or a PDGF-C antagonist is to be used for therapeutic purposes, the dose(s) and route of administration will depend upon the nature of the patient and condition to be treated, and will be at the discretion of the attending physician or veterinarian. Suitable routes include oral, subcutaneous, intramuscular, intraperitoneal or intravenous injection, parenteral, topical application, implants etc. Topical application of PDGF-C may be used in a manner analogous to VEGF. For example, where used for wound healing or other use in which enhanced angiogenesis is advantageous, an effective amount of the truncated active form of PDGF-C is administered to an organism in need thereof in a dose between about 0.1 and 1000 μg/kg body weight.

The PDGF-C or a PDGF-C antagonist may be employed in combination with a suitable pharmaceutical carrier. The resulting compositions comprise a therapeutically effective amount of PDGF-C or a PDGF-C antagonist, and a pharmaceutically acceptable non-toxic salt thereof, and a pharmaceutically acceptable solid or liquid carrier or adjuvant. Examples of such a carrier or adjuvant include, but are not limited to, saline, buffered saline, Ringer's solution, mineral oil, talc, corn starch, gelatin, lactose, sucrose, microcrystalline cellulose, kaolin, mannitol, dicalcium phosphate, sodium chloride, alginic acid, dextrose, water, glycerol, ethanol, thickeners, stabilizers, suspending agents and combinations thereof. Such compositions may be in the form of solutions, suspensions, tablets, capsules, creams, salves, elixirs, syrups, wafers, ointments or other conventional forms. The formulation to suit the mode of administration. Compositions which comprise PDGF-C may optionally further comprise one or more of PDGF-A, PDGF-B, VEGF, VEGF-B, VEGF-C, VEGF-D, PlGF and/or heparin. Compositions comprising PDGF-C will contain from about 0.1% to 90% by weight of the active compound(s), and most generally from about 10% to 30%.

For intramuscular preparations, a sterile formulation, preferably a suitable soluble salt form of the truncated active form of PDGF-C, such as hydrochloride salt, can be dissolved and administered in a pharmaceutical diluent such as pyrogen-free water (distilled), physiological saline or 5% glucose solution. A suitable insoluble form of the compound may be prepared and administered as a suspension in an aqueous base or a pharmaceutically acceptable oil base, e.g. an ester of a long chain fatty acid such as ethyl oleate.

According to yet a further aspect, the invention provides diagnostic/prognostic devices typically in the form of test kits. For example, in one embodiment of the invention there is provided a diagnostic/prognostic test kit comprising an antibody to PDGF-C and a means for detecting, and more preferably evaluating, binding between the antibody and PDGF-C. In one preferred embodiment of the diagnostic/prognostic device according to the invention, a second antibody (the secondary antibody) directed against antibodies of the same isotype and animal source of the antibody directed against PDGF-C (the primary antibody) is provided. The secondary antibody is coupled to a detectable label, and then either an unlabeled primary antibody or PDGF-C is substrate-bound so that the PDGF-C/primary antibody interaction can be established by determining the amount of label bound to the substrate following binding between the primary antibody and PDGF-C and the subsequent binding of the labeled secondary antibody to the primary antibody. In a particularly preferred embodiment of the invention, the diagnostic/prognostic device may be provided as a conventional enzyme-linked immunosorbent assay (ELISA) kit.

In another alternative embodiment, a diagnostic/prognostic device may comprise polymerase chain reaction means for establishing sequence differences of a PDGF-C of a test individual and comparing this sequence structure with that disclosed in this application in order to detect any abnormalities, with a view to establishing whether any aberrations in PDGF-C expression are related to a given disease condition.

In addition, a diagnostic/prognostic device may comprise a restriction length polymorphism (RFLP)generating means utilizing restriction enzymes and genomic DNA from a test individual to generate a pattern of DNA bands on a gel and comparing this pattern with that disclosed in this application in order to detect any abnormalities, with a view to establishing whether any aberrations in PDGF-C expression are related to a given disease condition.

In accordance with a further aspect, the invention relates to a method of detecting aberrations in PDGF-C gene in a test subject which may be associated with a disease condition in the test subject. This method comprises providing a DNA or RNA sample from said test subject; contacting the DNA sample or RNA with a set of primers specific to PDGF-C DNA operatively coupled to a polymerase and selectively amplifying PDGF-C DNA from the sample by polymerase chain reaction, and comparing the nucleotide sequence of the amplified PDGF-C DNA from the sample with the nucleotide sequences shown in FIG. 1 (SEQ ID NO:2) or FIG. 3 (SEQ ID NO:5). The invention also includes the provision of a test kit comprising a pair of primers specific to PDGF-C DNA operatively coupled to a polymerase, whereby said polymerase is enabled to selectively amplify PDGF-C DNA from a DNA sample.

The invention also provides a method of detecting PDGF-C in a biological sample, comprising the step of contacting the sample with a reagent capable of binding PDGF-C, and detecting the binding. Preferably the reagent capable of binding PDGF-C is an antibody directed against PDGF-C, particularly preferably a monoclonal antibody. In a preferred embodiment the binding and/or extent of binding is detected by means of a detectable label; suitable labels are discussed above.

In another aspect, the invention relates to a protein dimer comprising the PDGF-C polypeptide, particularly a disulfide-linked dimer. The protein dimers of the invention include both homodimers of PDGF-C polypeptide and heterodimers of PDGF-C and VEGF, VEGF-B, VEGF-C, VEGF-D, PlGF, PDGF-A or PDGF-B.

According to a yet further aspect of the invention there is provided a method for isolation of PDGF-C comprising the step of exposing a cell which expresses PDGF-C to heparin to facilitate release of PDGF-C from the cell, and purifying the thus-released PDGF-C.

Another aspect of the invention involves providing a vector comprising an anti-sense nucleotide sequence which is complementary to at least a part of a DNA sequence which encodes PDGF-C or a fragment or analog thereof that has the biological activity of PDGF-C. In addition the anti-sense nucleotide sequence can be to the promoter region of the PDGF-C gene or other non-coding region of the gene which may be used to inhibit, or at least mitigate, PDGF-C expression.

According to a yet further aspect of the invention such a vector comprising an anti-sense sequence may be used to inhibit, or at least mitigate, PDGF-C expression. The use of a vector of this type to inhibit PDGF-C expression is favored in instances where PDGF-C expression is associated with a disease, for example where tumors produce PDGF-C in order to provide for angiogenesis, or tissue remodeling that takes place during invasion of tumor cells into a normal cell population by primary or metastatic tumor formation. Transformation of such tumor cells with a vector containing an anti-sense nucleotide sequence would inhibit or retard growth of the tumor or tissue remodeling.

Another aspect of the invention relates to the discovery that the full length PDGF-C protein is likely to be a latent growth factor that needs to be activated by proteolytic processing to release an active PDGF/VEGF homology domain. A putative proteolytic site is found in residues 231-234 in the full length protein, residues —RKSR—. This is a dibasic motif. This site is structurally conserved in the mouse PDGF-C. The —RKSR— putative proteolytic site is also found in PDGF-A, PDGF-B, VEGF-C and VEGF-D. In these four proteins, the putative proteolytic site is also found just before the minimal domain for the PDGF/VEGF homology domain. Together these facts indicate that this is the proteolytic site.

Preferred proteases include, but are not limited, to plasmin, Factor X and enterokinase. The N-terminal CUB domain may function as an inhibitory domain which might be used to keep PDGF-C in a latent form in some extracellular compartment and which is removed by limited proteolysis when PDGF-C is needed.

According to this aspect of the invention, a method is provided for producing an activated truncated form of PDGF-C or for regulating receptor-binding specificity of PDGF-C. These methods comprise the steps of expressing an expression vector comprising a polynucleotide encoding a polypeptide having the biological activity of PDGF-C and supplying a proteolytic amount of at least one enzyme for processing the expressed polypeptide to generate the activated truncated form of PDGF-C.

This aspect also includes a method for selectively activating a polypeptide having a growth factor activity. This method comprises the step expressing an expression vector comprising a polynucleotide encoding a polypeptide having a growth factor activity, a CUB domain and a proteolytic site between the polypeptide and the CUB domain, and supplying a proteolytic amount of at least one enzyme for processing the expressed polypeptide to generate the activated polypeptide having a growth factor activity.

In addition, this aspect includes the isolation of a nucleic acid molecule which codes for a polypeptide having the biological activity of PDGF-C and a polypeptide thereof which comprises a proteolytic site having the amino acid sequence RKSR or a structurally conserved amino acid sequence thereof.

Also this aspect includes an isolated dimer comprising an activated monomer of PDGF-C and an activated monomer of VEGF, VEGF-B, VEGF-C, VEGF-D, PDGF-C, PDGF-A, PDGF-B or PlGF linked to a CUB domain, or alternatively, an activated monomer of VEGF, VEGF-B, VEGF-C, VEGF-D, PDGF-C, PDGF-A, PDGF-B or PlGF and an activated monomer of PDGF-C linked to a CUB domain. The isolated dimer may or may not include a proteolytic site between the activator monomer and the CUB domain linkage.

Polynucleotides of the invention such as those described above, fragments of those polynucleotides, and variants of those polynucleotides with sufficient similarity to the non-coding strand of those polynucleotides to hybridize thereto under stringent conditions all are useful for identifying, purifying, and isolating polynucleotides encoding other, non-human, mammalian forms of PDGF-C. Thus, such polynucleotide fragments and variants are intended as aspects of the invention. Exemplary stringent hybridization conditions are as follows: hybridization at 42° C. in 5×SSC, 20 mM NaPO₄, pH 6.8, 50% formamide; and washing at 42° C. in 0.2×SSC. Those skilled in the art understand that it is desirable to vary these conditions empirically based on the length and the GC nucleotide base content of the sequences to be hybridized, and that formulas for determining such variation exist. See for example Sambrook et al, “Molecular Cloning: A Laboratory Manual”, Second Edition, pages 9.47-9.51, Cold Spring Harbor, N.Y.: Cold Spring Harbor Laboratory (1989).

Moreover, purified and isolated polynucleotides encoding other, non-human, mammalian PDGF-C forms also are aspects of the invention, as are the polypeptides encoded thereby and antibodies that are specifically immunoreactive with the non-human PDGF-C variants. Thus, the invention includes a purified and isolated mammalian PDGF-C polypeptide and also a purified and isolated polynucleotide encoding such a polypeptide.

It will be clearly understood that nucleic acids and polypeptides of the invention may be prepared by synthetic means or by recombinant means, or may be purified from natural sources.

It will be clearly understood that for the purposes of this specification the word “comprising” means “included but not limited to”. The corresponding meaning applies to the word “comprises”.

### BRIEF DESCRIPTION OF THE DRAWINGS

FIG. 1 (SEQ ID NO:2) shows the complete nucleotide sequence of cDNA encoding a human PDGF-C (hPDGF-C)(2108 bp);

FIG. 2 (SEQ ID NO:3) shows the deduced amino acid sequence of full-length hPDGF-C which consists of 345 amino acid residues (the translated part of the cDNA corresponds to nucleotides 37 to 1071 of FIG. 1);

FIG. 3 (SEQ ID NO:4) shows a cDNA sequence encoding a fragment of human PDGF-C (hPDGF-C)(1536 bp);

FIG. 4 (SEQ ID NO:5) shows a deduced amino acid sequence of a fragment of hPDGF-C(translation of nucleotides 3 to 956 of the nucleotide sequence of FIG. 3);

FIG. 5 (SEQ ID NO:6) shows a nucleotide sequence of a murine PDGF-C (mPDGF-C) cDNA;

FIG. 6 (SEQ ID NO:7) shows the deduced amino acid sequence of a fragment of mPDGF-C(the translated part of the cDNA corresponds to nucleotides 196 to 1233 of FIG. 5);

FIG. 7 shows a comparative sequence alignment of the hPDGF-C amino acid sequence of FIG. 2 (SEQ ID NO:3) with the mPDGF-C amino acid sequence of FIG. 6 (SEQ ID NO:7);

FIG. 8 shows a schematic structure of mPDGF-C with a signal sequence (striped box), a N-terminal C1r/C1s/embryonic sea urchin protein Uegf/bone morphogenetic protein 1 (CUB) domain and the C-terminal PDGF/VEGF-homology domain (open boxes);

FIG. 9 shows a comparative sequence alignment of the PDGF/VEGF-homology domains in human and mouse PDGF-C with other members of the VEGF/PDGF family of growth factors (SEQ ID NOs:8-17, respectively);

FIG. 10 shows a phylogenetic tree of several growth factors belonging to the VEGF/PDGF family;

FIG. 11 provides the amino acid sequence alignment of the CUB domain present in human and mouse PDGF-Cs (SEQ ID NOs:18 and 19, respectively) and other CUB domains present in human bone morphogenic protein-1 (hBMP-1, 3 CUB domains CUB1-3)(SEQ ID NOs:20-22, respectively) and in human neuropilin-1 (2 CUB domains)(SEQ ID NOs:23 and 24, respectively);

FIG. 12 shows a Northern blot analysis of the expression of PDGF-C transcripts in several human tissues;

FIG. 13 shows the regulation of PDGF-C mRNA expression by hypoxia; and

FIG. 14 shows the expression of PDGF-C in human tumor cell lines.

FIG. 15 shows the results of immunoblot detection of full length human PDGF-C in transfected COS-1 cells.

FIG. 16 shows isolation and partial characterization of full length PDGF-C.

FIG. 17 shows isolation and partial characterization of a truncated form of human PDGF-C containing the PDGF/VEGF homology domain only.

FIG. 18 provides a standard curve for the binding of labeled PDGF-BB homodimers to PAE-1 cells expressing PDGF alpha receptor.

FIG. 19 provides a graphic representation of the inhibition of binding of labeled PDGF-BB to PAE-1 cells expressing PDGF alpha receptor by increasing amounts of purified full length and truncated PDGF-CC proteins.

FIG. 20 shows the effects of the full length and truncated PDGF-CC homodimers on the phosphorylation of PDGF alpha-receptor.

FIG. 21 shows the mitogenic activities of the full length and truncated PDGF-CC homodimers on fibroblasts.

FIG. 22 graphically presents the results of the binding assay of truncated PDGF-C to the PDGF receptors.

FIG. 23 shows the immunoblot of the undigested full length PDGF-C protein and the plasmin-generated 26-28 kDa species.

FIG. 24 graphically presents the results of the competitive binding assay of full-length PDGF-C and truncated PDGF-C for PDGFR-alpha receptors.

FIG. 25 shows the analyses by SDS-PAGE of the human PDGF-C CUB domain under reducing and non-reducing conditions.

FIGS. 26A-26V show PDGF-C expression in the developing mouse embryo.

FIGS. 27A-27F show PDGF-C, PDGF-A and PDGFR-alpha expression in the developing kidney.

FIGS. 28A-28F show histology of E 16.5 kidneys from wildtype (FIGS. 28A and 28C), PDGFR-alpha −/− (FIGS. 28B and 28F, PDGF-A −/− (FIG. 28D) and PDGF-A/PDGF-B double −/− (FIG. 28E) kidneys.

FIG. 29 shows a polyacrylamide gel analysis of dimeric and monomeric forms of PDGF-C.

FIGS. 30A-D show results of a chick embryo chorioallantoic membrane assay demonstrating stimulation of angiogenesis and vessel sprouts by PDGF-CC.

FIGS. 31A-G show a comparison of corneal neovascularization induced by PDGF-CC, FGF-2 and VEGF.

FIGS. 32A-G show a comparison of angiogenic responses induced by various members of the PDGF growth factor family.

FIGS. 33A-E show the results of immunochemical analyses of mouse corneas implanted with members of the PDGF family.

### DETAILED DESCRIPTION OF PREFERRED EMBODIMENTS

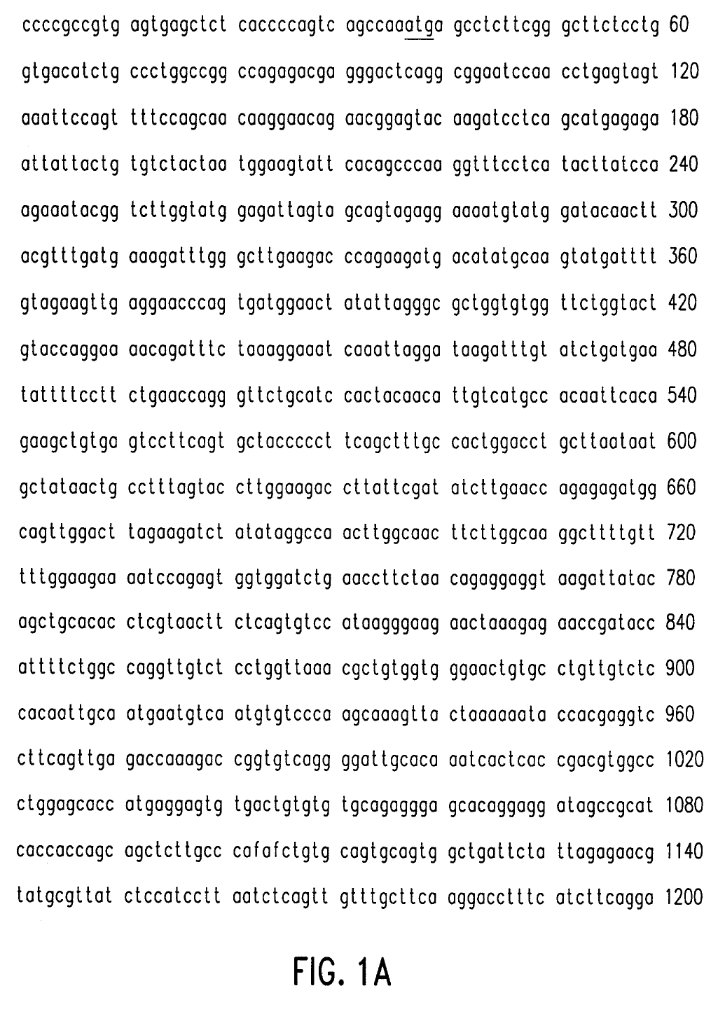

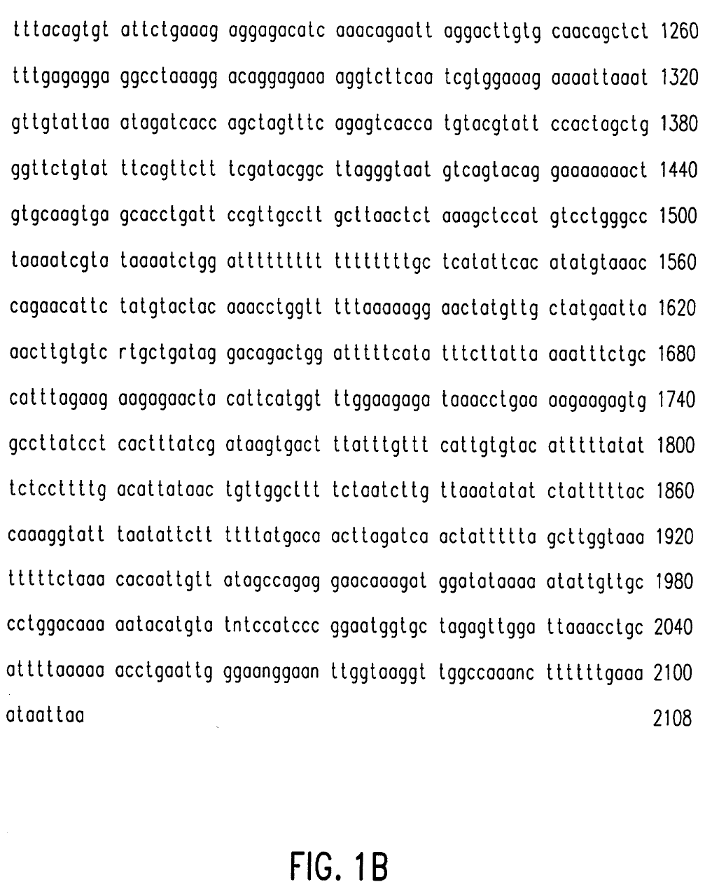

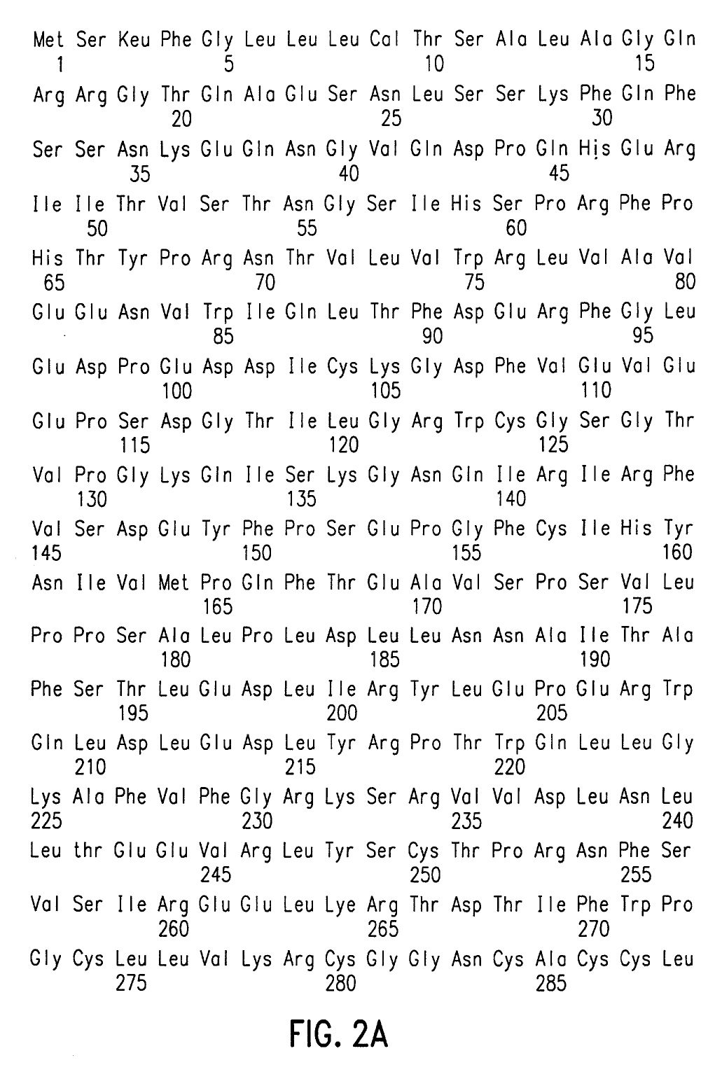

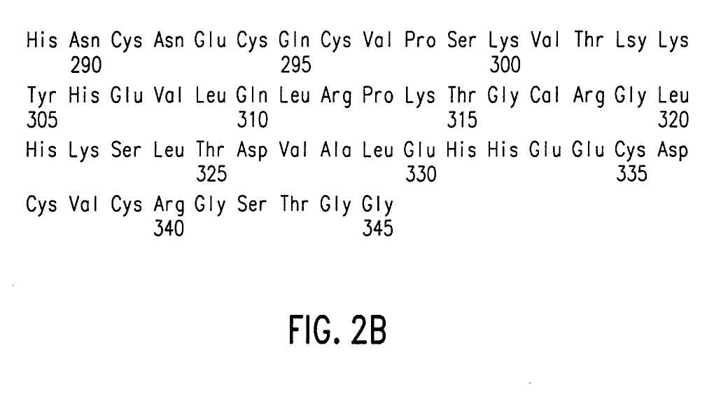

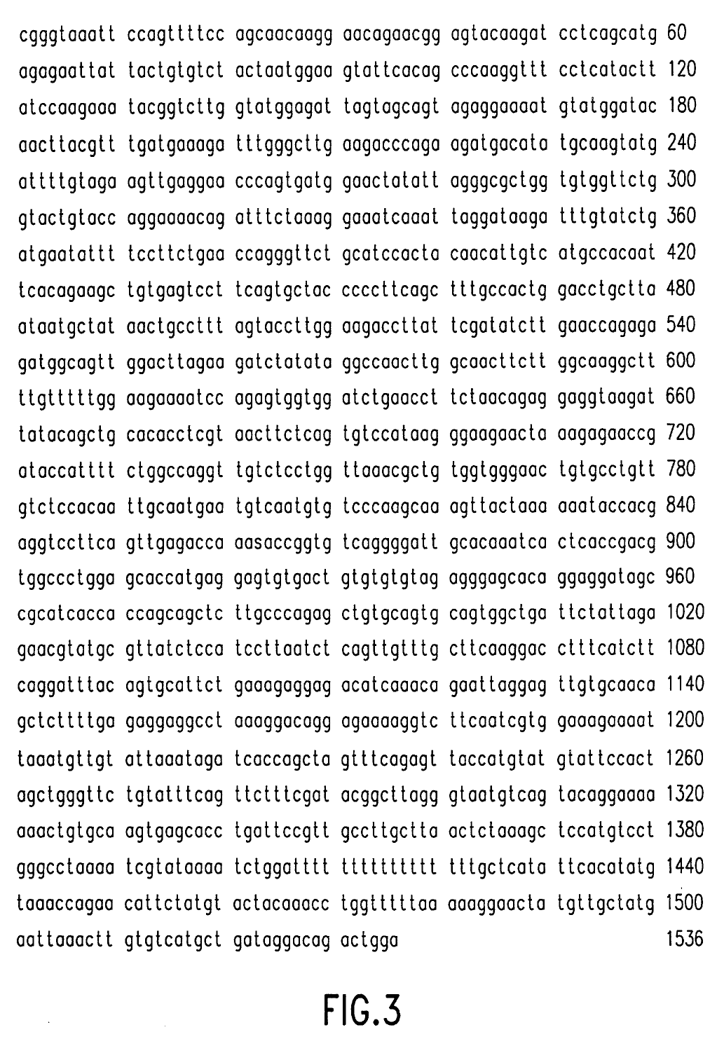

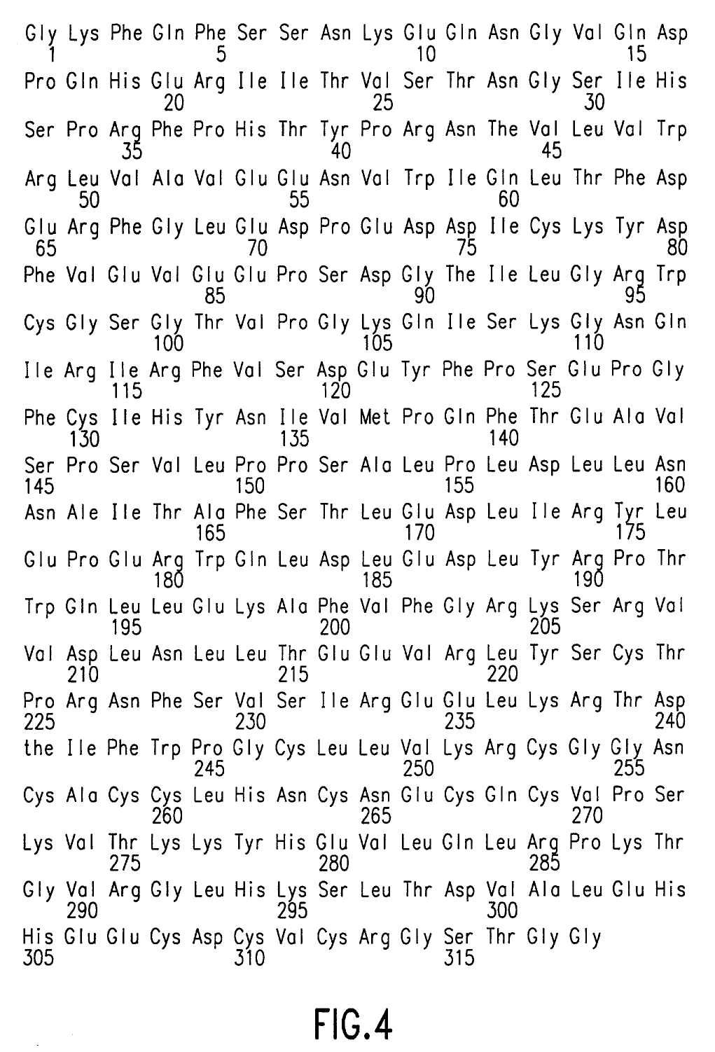

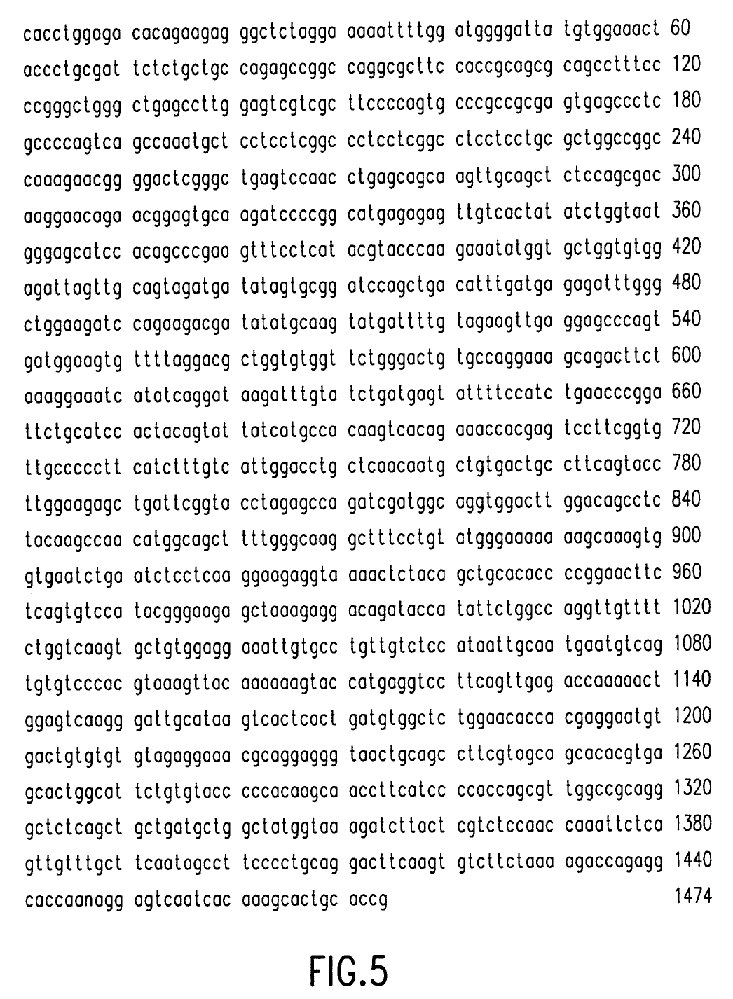

FIG. 1 (SEQ ID NO:2) shows the complete nucleotide sequence of cDNA encoding a human PDGF-C (hPDGF-C)(2108 bp), which is a new member of the VEGF/PDGF family. A clone #4 (see FIGS. 3 and 4—SEQ ID NOs:4 and 5) encoding hPDGF-C was not full length and lacked approximately 80 base pairs of coding sequence when compared to the mouse protein (corresponding to 27 amino acids). Additional cDNA clones were isolated from a human fetal lung cDNA library to obtain an insert which included this missing sequence. Clone #10 had a longer insert than clone #4. The insert of clone #10 was sequenced in the 5′ region and it was found to contain the missing sequence. Clone #10 was found to include the full sequence of human PDGF-C. Some 5′-untranslated sequence, the translated part of the cDNA encoding human PDGF-C and some 3′-untranslated nucleotide sequence are shown in FIG. 1 (SEQ ID NO:2). A stop codon in frame is located 21 bp upstream of the initiation ATG (the initiation ATG is underlined in FIG. 1).

Work to isolate this new human PDGF/VEGF began after a search of the expressed sequence tag (EST) database, dbEST, at the National Center for Biotechnology Information (NCBI) in Washington, DC, identified a human EST sequence (W21436) which appears to encode part of the human homolog of the mouse PDGF-C. Based on the human EST sequence, two oligonucleotides were designed:

5′-GAA GTT GAG GAA CCC AGT G-3′ forward (SEQ ID NO:25)

5′-CTT GCC AAG AAG TTG CCA AG-3′ reverse (SEQ ID NO:26).

These oligonucleotides were used to amplify by polymerase chain reaction (PCR) a polynucleotide of 348 bps from a Human Fetal Lung 5′-STRETCH PLUS λgt10 cDNA library, which was obtained commercially from Clontech. The PCR product was cloned into the pCR 2.1-vector of the Original TA Cloning Kit (Invitrogen). Subsequently, the 348 bps cloned PCR product was used to construct a hPDGF-C probe according to standard techniques.

10⁶ lambda-clones of the Human Fetal Lung 5′-STRETCH PLUS λgt10 cDNA Library (Clontech) were screened with the hPDGF-C probe according to standard procedures. Among several positive clones, one, clone #4 was analyzed more carefully and the nucleotide sequence of its insert was determined according to standard procedures using internal and vector oligonucleotides. The insert of clone #4 contains a partial nucleotide sequence of the cDNA encoding the full length human PDGF-C (hPDGF-C). The nucleotide sequence (1536 bp) of the clone #4 insert is shown in FIG. 3 (SEQ ID NO:4). The translated portion of this cDNA includes nucleotides 6 to 956. The deduced amino acid sequence of the translated portion of the insert is illustrated in FIG. 4 (SEQ ID NO:5). A polypeptide of this deduced amino acid sequence would lack the first 28 amino acid residues found in the full length hPDGF-C polypeptide. However, this polypeptide includes a proteolytic fragment which is sufficient to activate the PDGF alpha receptors. It should be noted that the first glycine (Gly) of SEQ ID NO:5 is not found in the full length hPDGF-C.

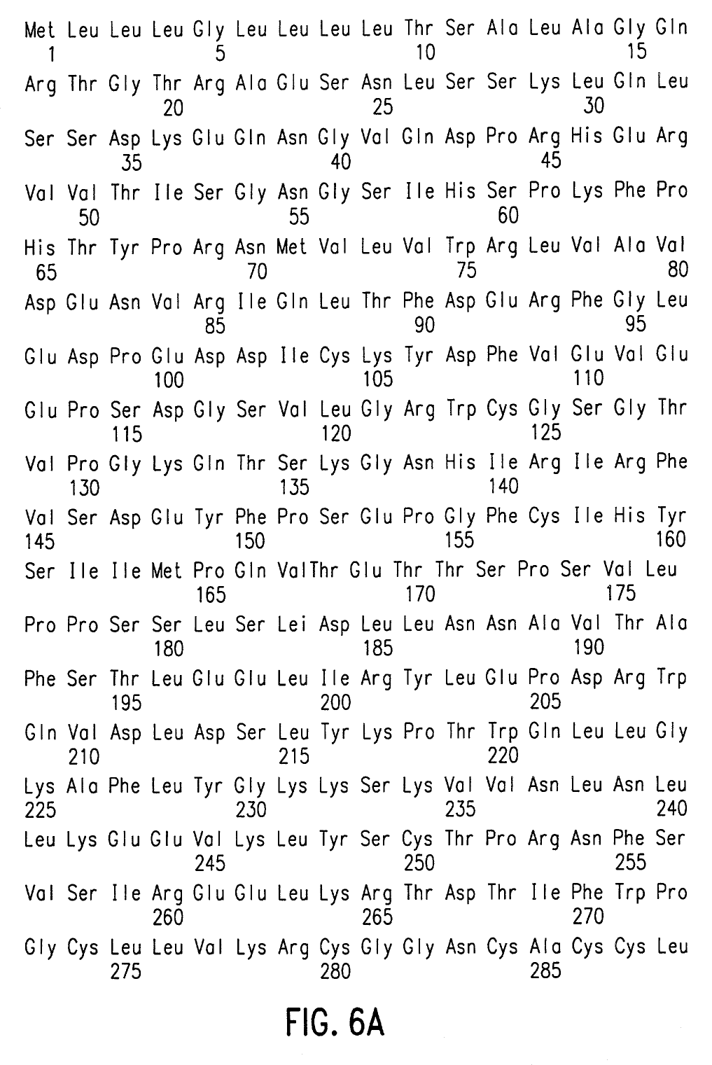

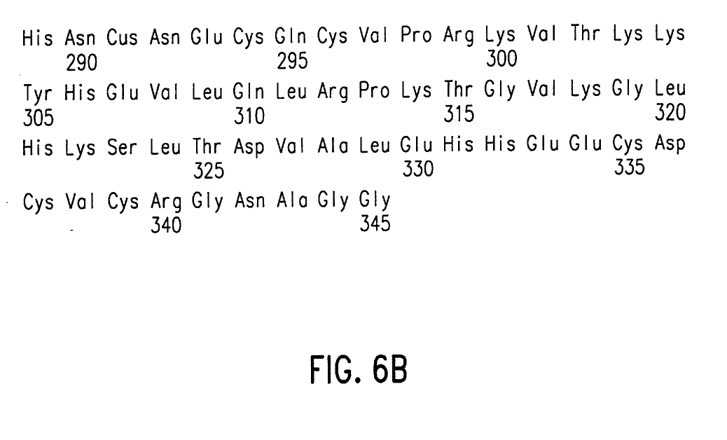

A mouse EST sequence (AI020581) was identified in a database search of the dbEST database at the NCBI in Washington, D.C., which appears to encode part of a new mouse PDGF, PDGF-C. Large parts of the mouse cDNA was obtained by PCR amplification using DNA from a mouse embryo λgt10 cDNA library as the template. To amplify the 3′ end of the cDNA, a sense primer derived from the mouse EST sequence was used (the sequence of this primer was 5′-CTT CAG TAC CTT GGA AGA G, primer 1 (SEQ ID NO:27)) To amplify the 5′ end of the cDNA, an antisense primer derived from the mouse EST was used (the sequence of this primer was 5′-CGC TTG ACC AGG AGA CAA C, primer 2 (SEQ ID NO:28)). The λgt10 vector primers were sense 5′-ACG TGA ATT CAG CAA GTT CAG CCT GGT TAA (primer 3 (SEQ ID NO:29)) and antisense 5′-ACG TGG ATC CTG AGT ATT TCT TCC AGG GTA (primer 4 (SEQ ID NO:30)). Combinations of the vector primers and the internal primers obtained from the mouse EST were used in standard PCR reactions. The sizes of the amplified fragments were approx. 750 bp (3′-fragment) and 800 bp (5′-fragment), respectively. These fragments were cloned into the pCR 2.1 vector and subjected to nucleotide sequences analysis using vector primers and internal primers. Since these fragments did not contain the full length sequence of mPDGF-C, a mouse liver ZAP cDNA library was screened using standard conditions. A 261 bp ³²P-labeled PCR fragment was generated for use as a probe using primers 1 and 2 and using DNA from the mouse embryo λgt10 library as the template (see above). Several positive plaques were purified and the nucleotide sequence of the inserts were obtained following subcloning into pBluescript. Vector specific primers and internal primers were used. By combining the nucleotide sequence information of the generated PCR clones and the isolated clone, the full length amino acid sequence of mPDGF-C could be deduced (see FIG. 6)(SEQ ID NO:7).

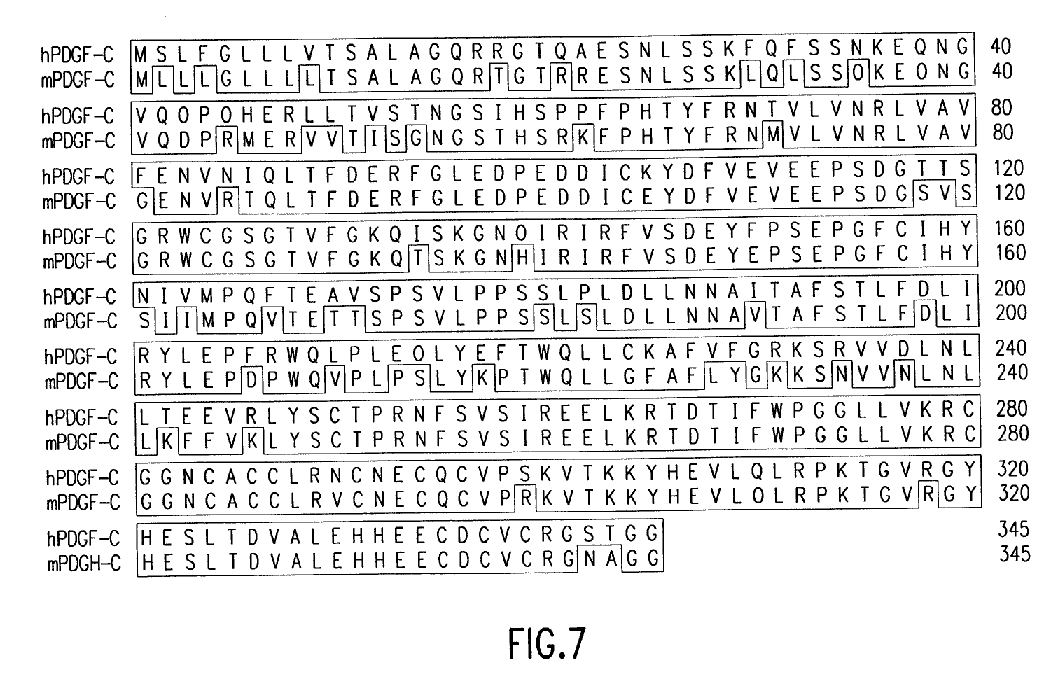

FIG. 7 shows a comparative sequence alignment of the mouse and human amino acid sequences of PDGF-C (SEQ ID NOS:6 and 2, respectively). The alignment shows that human and mouse PDGF-Cs display an identity of about 87% with 45 amino acid replacements found among the 345 residues of the full length proteins. Almost all of the observed amino acid replacements are conservative in nature. The predicted cleavage site in mPDGF-C for the signal peptidase is between residues G19 and T20. This would generate a secreted mouse peptide of 326 amino acid residues.

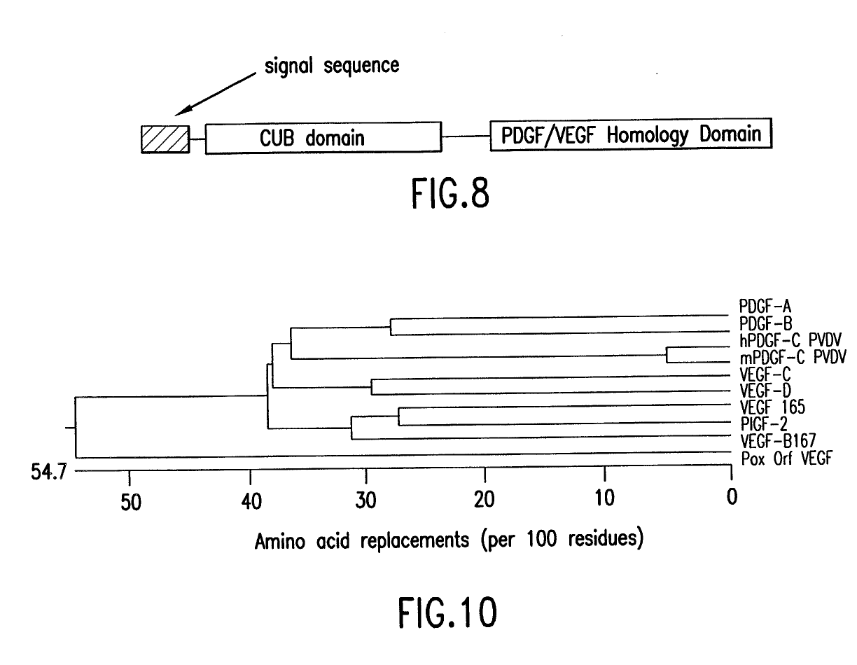

FIG. 8 provides a schematic domain structure of mouse PDGF-C with a signal sequence (striped box), a N-terminal CUB domain and the C-terminal PDGF/VEGF-homology domain (open boxes). The amino acid sequences denoted by the lines have no obvious similarities to CUB domains or to VEGF-homology domains.

The high sequence identity suggests that human and mouse PDGF-C have an almost identical domain structure. Amino acid sequence comparisons revealed that both mouse and human PDGF-C display a novel domain structure. Apart from the PDGF/VEGF-homology domain located in the C-terminal region in both proteins (residues 164 to 345), the N-terminal region in both PDGF-Cs have a domain referred to as a CUB domain (Bork and Beckmann, J. Mol. Biol., 1993 231, 539-545). This domain of about 110 amino acids (amino acid residues 50-160) was originally identified in complement factors C1r/C1s, but has recently been identified in several other extracellular proteins including signaling molecules such as bone morphogenic protein 1 (BMP-1) (Wozney et al., Science, 1988 242, 1528-1534) as well as in several receptor molecules such as neuropilin-1 (NP-1) (Soker et al., Cell, 1998 92 735-745). The functional roles of CUB domains are not clear but it may participate in protein-protein interactions or in interactions with carbohydrates including heparin sulfate proteoglycans.

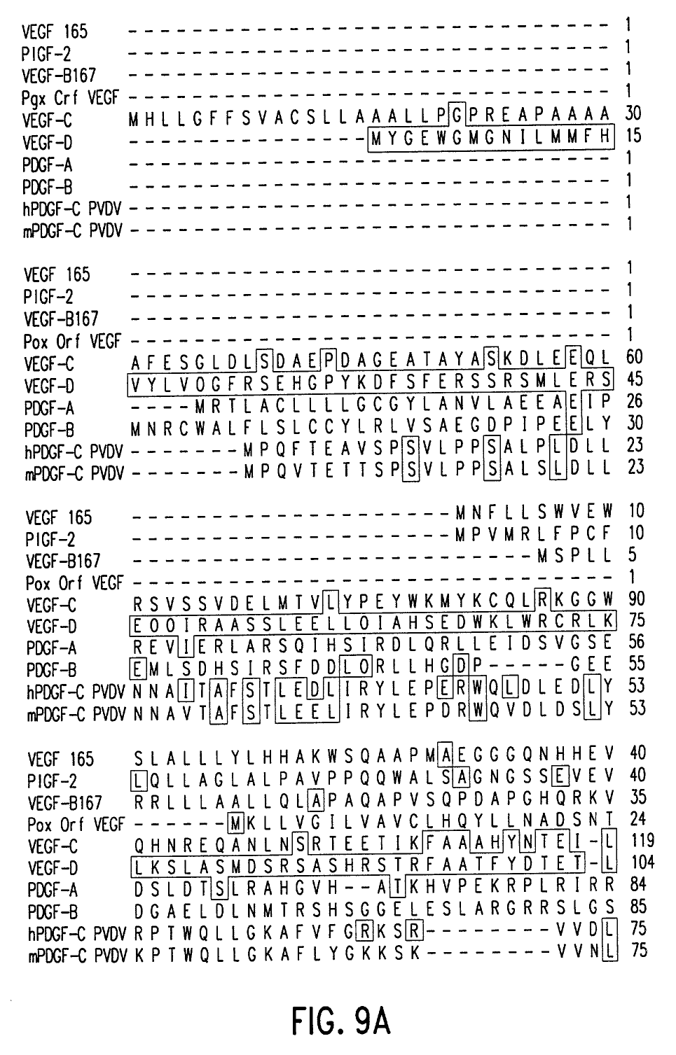

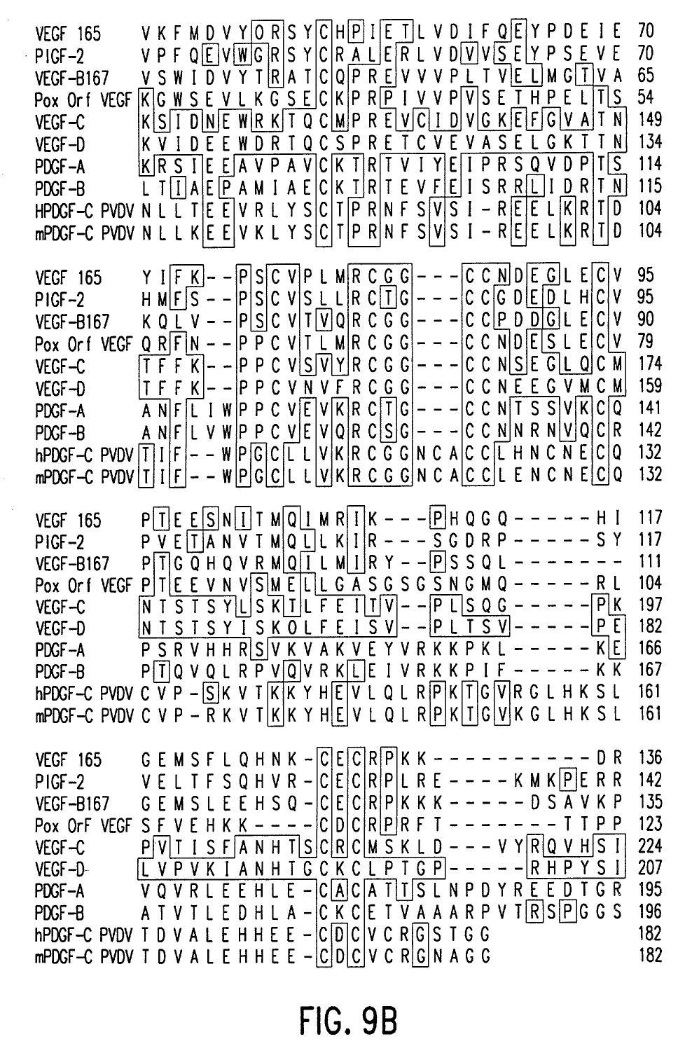

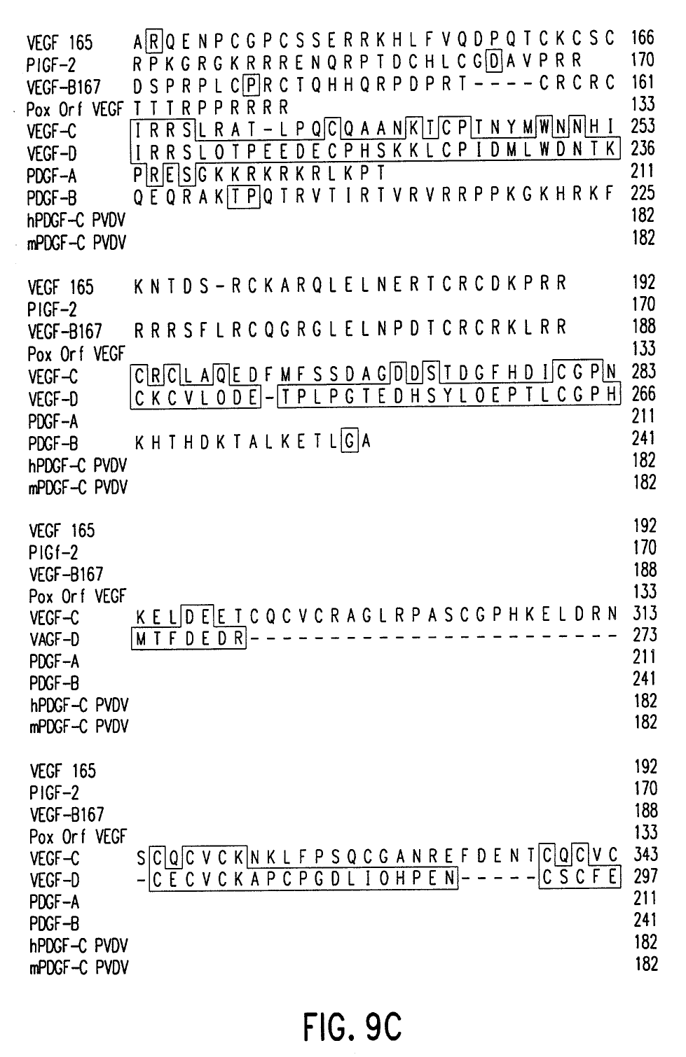

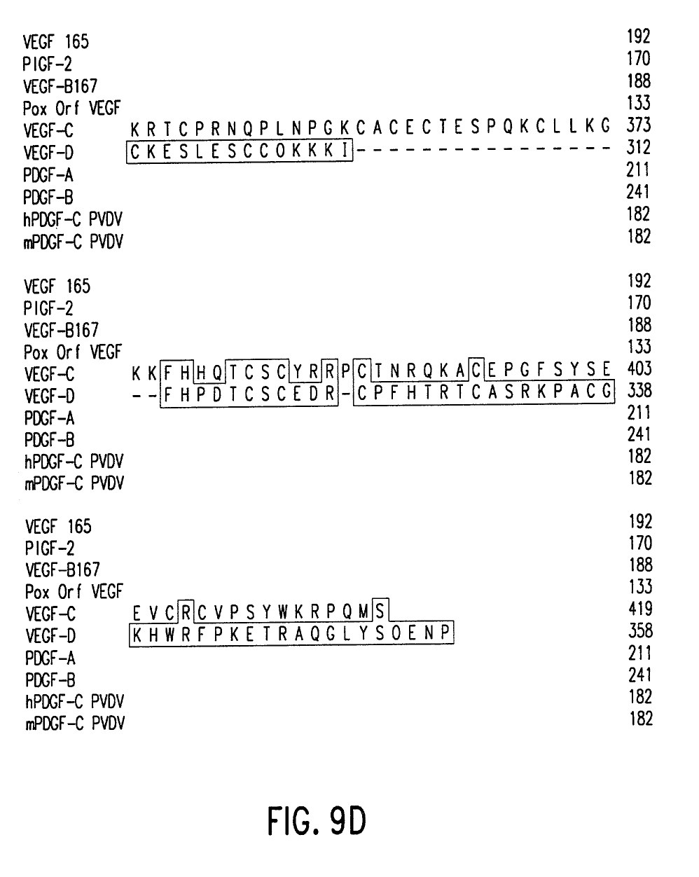

FIG. 9 shows the amino acid sequence alignment of the C-terminal PDGF/VEGF-homology domains of human and mouse PDGF-Cs with the C-terminal PDGF/VEGF-homology domains of PDGF/VEGF family members, VEGF₁₆₅, PlGF-2, VEGF-B₁₆₇, Pox Orf VEGF, VEGF-C, VEGF-D, PDGF-A and PDGF-B (SEQ ID NOs:8-17). Some of the amino acid sequences in the N- and C-terminal regions in VEGF-C and VEGF-D have been deleted in this figure. Gaps were introduced to optimize the alignment. This alignment was generated using the method of J. Hein, (Methods Enzymol. 1990 183 626-45) with PAM250 residue weight table. The boxed residues indicate amino acids which match the PDGF-Cs within two distance units.

The alignment shows that PDGF-C has the expected pattern of invariant cysteine residues, a hallmark of members of this family, with one exception. Between cysteine 3 and 4, normally spaced by 2 residues there is an insertion of three extra amino acids (NCA). This feature of the sequence in PDGF-C was highly unexpected.

Based on the amino acid sequence alignments in FIG. 9, a phylogenetic tree was constructed and is shown in FIG. 10. The data show that the PDGF-C homology domain is closely related to the PDGF/VEGF-homology domains of VEGF-C and VEGF-D.

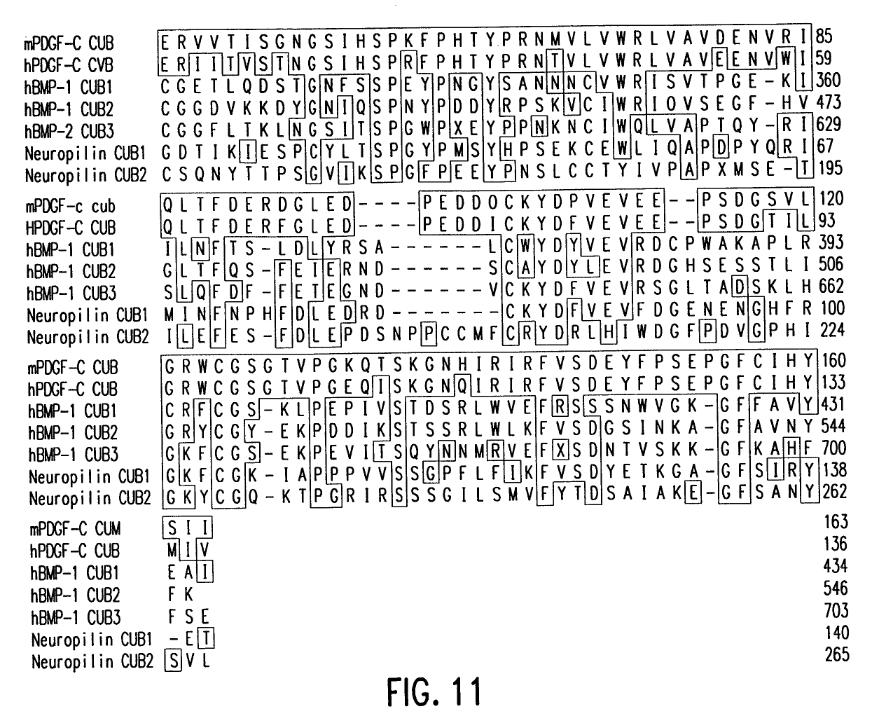

As shown in FIG. 11, the amino acid sequences from several CUB-containing proteins were aligned (SEQ ID NOs:18-24). The results show that the single CUB domain in human and mouse PDGF-C (SEQ ID NOs:18 and 19, respectively) displays a significant identify with the most closely related CUB domains. Sequences from human BMP-1, with 3 CUB domains (CUB1-3 (SEQ ID NOs:20-22)) and human neuropilin-1 with 2 CUB domains (CUB1-2) (SEQ ID NOs:23 and 24, respectively) are shown. Gaps were introduced to optimize the alignment. This alignment was generated using the method of J. Hein, (Methods Enzymol., 1990 183 626-45) with PAM250 residue weight table.

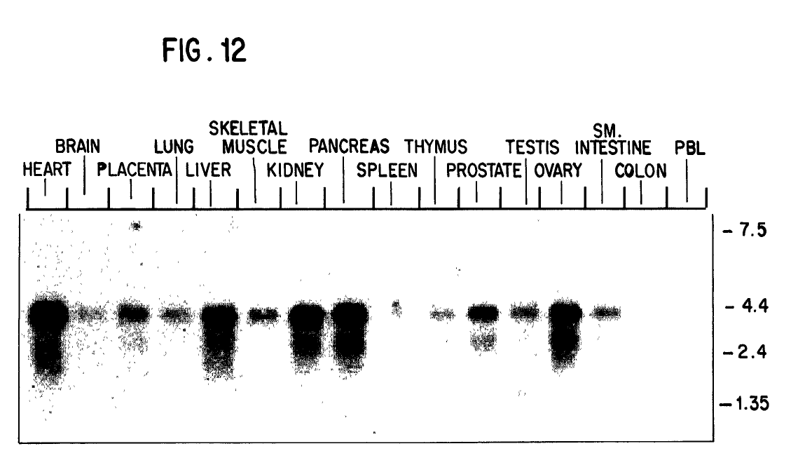

FIG. 12 shows a Northern blot analysis of the expression of PDGF-C transcripts in several human tissues. The analysis shows that PDGF-C is encoded by a major transcript of approximately 3.8-3.9 kb, and a minor of 2.8 kb. The numbers to the right refer to the size of the mRNAs (in kb). The tissue expression of PDGF-C was determined by Northern blotting using a commercial Multiple Tissue Northern blot (MTN, Clontech). The blots were hybridized at according to the instructions from the supplier using ExpressHyb solution at 68° C. for one hour (high stringency conditions), and probed with a 353 bp hPDGF-C EST probe from the fetal lung cDNA library screening as described above. The blots were subsequently washed at 50° C. in 2×SSC with 0.05% SDS for 30 minutes and at 50° C. in 0.1×SSC with 0.1% SDS for an additional 40 minutes. The blots were then put on film and exposed at −70° C. The blots show that PDGF-C transcripts are most abundant in heart, liver, kidney, pancreas and ovary while lower levels of transcripts are present in most other tissues, including placenta, skeletal muscle and prostate. PDGF-C transcripts were below the level of detection in spleen, colon and peripheral blood leucocytes.

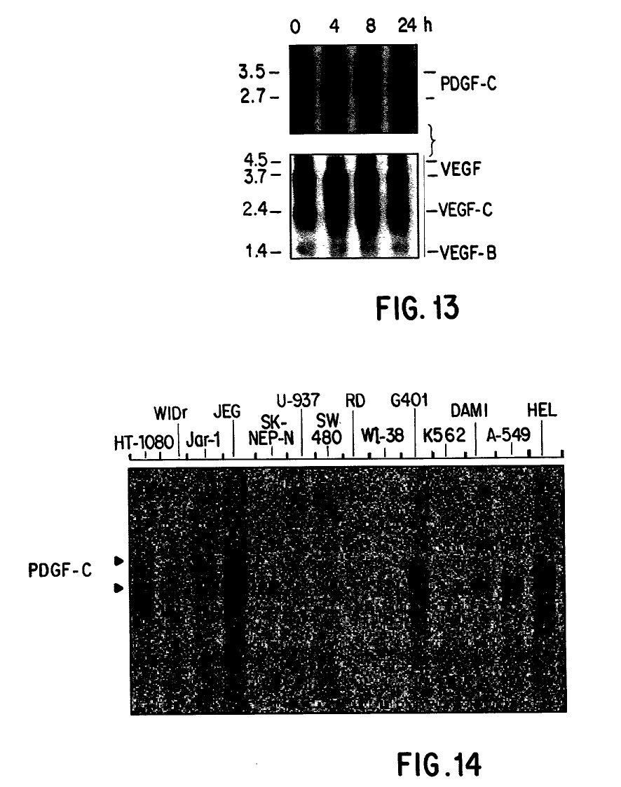

FIG. 13 shows the regulation of PDGF-C mRNA expression by hypoxia. Size markers (in kb) are indicated to the left in the lower panel. The estimated sizes of PDGF-C mRNAs is indicated to the left in the upper panel (2.7 and 3.5 kbs, respectively). To explore whether PDGF-C is induced by hypoxia, cultured human skin fibroblasts were exposed to hypoxia for 0, 4, 8 and 24 hours. Poly(A)+mRNA was isolated from cells using oligo-dT cellulose affinity purification. Isolated mRNAs were electrophoresed through 12% agarose gels using 4 μg of mRNA per line. A Northern blot was made and hybridized with a probe for PDGF-C. The sizes of the two bands were determined by hybridizing the same filter with a mixture of hVEGF, hVEGF-B and hVEGF-C probes (Enholm et al. Oncogene, 1997 14 2475-2483), and interpolating on the basis of the known sizes of these mRNAs. The results shown in FIG. 13 indicate that PDGF-C is not regulated by hypoxia in human skin fibroblasts.

FIG. 14 shows the expression of PDGF-C mRNA in human tumor cells lines. To explore whether PDGF-C was expressed in human tumor cell lines, poly(A)+mRNA was isolated from several known tumor cell lines, the mRNAs were electrophoresed through a 12% agarose gel and analyzed by Northern blotting and hybridization with the PDGF-C probe. The results shown in FIG. 14 demonstrate that PDGF-C mRNA is expressed in several types of human tumor cell lines such as JEG3 (a human choriocarcinoma, ATCC #HTB-36), G401 (a Wilms tumor, ATCC #CRL-1441), DAMI (a megakaryoblastic leukemia), A549 (a human lung carcinoma, ATCC #CCL-185) and HEL (a human erythroleukemia, ATCC #TID-180). It is contemplated that further growth of these PDGF-C expressing tumors can be inhibited by inhibiting PDGF-C. As well as using PDGF-C expression as a means of identifying specific types of tumors.

### Example 1

### Generation of Specific Antipeptide Antibodies to Human PDGF-C

Two synthetic peptides were generated and then used to raise antibodies against human PDGF-C. The first synthetic peptide corresponds to residues 29-48 of the N-terminus of full length PDGF-C and includes an extra cysteine residue at the N- and C-terminus: CKFQFSSNKEQNGVQDPQHERC (SEQ ID NO:31). The second synthetic peptide corresponds to residues 230-250 of the internal region of full length PDGF-C and includes an extra cysteine residue at the C-terminus: GRKSRVVDLNLLTEEVRLYSC (SEQ ID NO:32). The two peptides were each conjugated to the carrier protein keyhole limpet hemocyanin (KLH, Calbiochem) using N-succinimidyl 3-(2-pyridyldithio)propionate (SPDP) (Pharmacia Inc.) according to the instructions of the supplier. 200-300 micrograms of the conjugates in phosphate buffered saline (PBS) were separately emulsified in Freunds Complete Adjuvant and injected subcutaneously at multiple sites in rabbits. The rabbits were boostered subcutaneously at biweekly intervals with the same amount of the conjugates emulsified in Freunds Incomplete Adjuvant. Blood was drawn and collected from the rabbits. The sera were prepared using standard procedures known to those skilled in the art.

### Example 2

### Expression of Full Length Human PDGF-C in Mammalian Cells

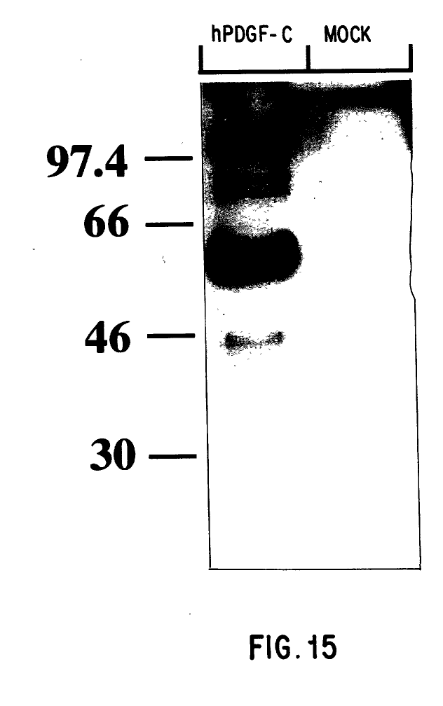

The full length cDNA encoding human PDGF-C was cloned into the mammalian expression vector, pSG5 (Stratagene, La Jolla, Calif.) that has the SV40 promoter. COS-1 cells were transfected with this construct and in separate transfections, with a pSG5 vector without the cDNA insert for a control, using the DEAE-dextran procedure. Serum free medium was added to the transfected COS-1 cells 24 hours after the transfections and aliquots containing the secreted proteins were collected for a 24 hour period after the addition of the medium. These aliquots were subjected to precipitation using ice cold 10% trichloroacetic acid for 30 minutes, and the precipitates were washed with acetone. The precipitated proteins were dissolved in SDS loading buffer under reducing conditions and separated on a SDS-PAGE gel using standard procedures. The separated proteins were electrotransferred onto Hybond filter and immunoblotted using a rabbit antiserum against the internal peptide of full length PDGF-C, the preparation of which is described above. Bound antibodies were detected using enhanced chemiluminescence (ECL, Amersham Inc.). FIG. 15 shows the results of this immunoblot. The sample was only partially reduced and the monomer of the human PDGF-C migrated as a 55 kDa species (the lower band) and the dimer migrated as a 100 kDa species (upper band). This indicates that the protein is secreted intact and that no major proteolytic processing occurs during secretion of the molecule in mammalian cells.

### Example 3

### Expression of Full Length and Truncated Human PDGF-C in Baculovirus Infected Sf9 Cells

The full length coding part of the human PDGF-C cDNA (970 bp) was amplified by PCR using Deep Vent DNA polymerase (Biolabs) using standard conditions and procedures. The full length PDGF-C was amplified for 30 cycles, where each cycle consisted of one minute denaturization at 94° C., one minute annealing at 56° C. and two minutes extension at 72° C. The forward primer used was 5′CGGGATCCCGAATCCAACCTGAGTAG3′ (SEQ ID NO:33). This primer includes a BamHI site (underlined) for in frame cloning. The reverse primer used was 5′GGAATTCCTAATGGTGATGGTGATGATGTTTGTCATCGTCATCTCCTCCTGTGCTC CCTCT3′ (SEQ ID NO:34). This primer includes an EcoRI site (underlined) and sequences coding for a C-terminal 6×His tag preceded by an enterokinase site. In addition, residues 230-345 of the PDGF/VEGF homology domain (PVHD) of human PDGF-C were amplified by PCR using Deep Vent DNA polymerase (Biolabs) using standard conditions and procedures. The residues 230-345 of the PVHD of PDGF-C were amplified for 25 cycles, where each cycle consisted of one minute denaturization at 94° C., four minutes annealing at 56° C. and four minutes extension at 72° C. The forward primer used was 5′CGGATCCCGGAAGAAAATCCA GAGTGGTG3′ (SEQ ID NO:35). This primer includes a BamHI site (underlined) for in frame cloning. The reverse primer used was 5′GGAATTCCTAATGGTGATGGTGATGATGTTTGTCATCGTCATCTCCTCCTGTG CTCCCTCT-3′ (SEQ ID NO:36). This primer includes an EcoRI site (underlined) and sequences coding for a C-terminal 6×His tag preceded by an enterokinase site. The PCR products were digested with BamHI and EcoRI and subsequently cloned into the baculovirus expression vector, pAcGP67A. Verification of the correct sequence of the PCR products cloned into the constructs was by nucleotide sequencing. The expression vectors were then co-transfected with BaculoGold linearized baculovirus DNA into Sf9 insect cells according to the manufactures protocol (Pharmingen). Recombined baculovirus were amplified several times before beginning large scale protein production and protein purification according to the manual (Pharmingen).

Sf9 cells, adapted to serum free medium, were infected with recombinant baculovirus at a multiplicity of infection of about 7. Media containing the recombinant proteins were harvested 4 days after infection and were incubated with Ni-NTA-Agarose beads (Qiagen). The beads were collected in a column and after extensive washing with 50 mM sodium phosphate buffer pH 8, containing 300 mM NaCl (the washing buffer), the bound proteins were eluted with increasing concentrations of imidazole (from 100 mM to 500 mM) in the washing buffer. The eluted proteins were analyzed by SDS-PAGE using 12.5% polyacrylamide gels under reducing and non-reducing conditions. For immunoblotting analyses, the proteins were electrotransferred onto Hybond filters for 45 minutes.

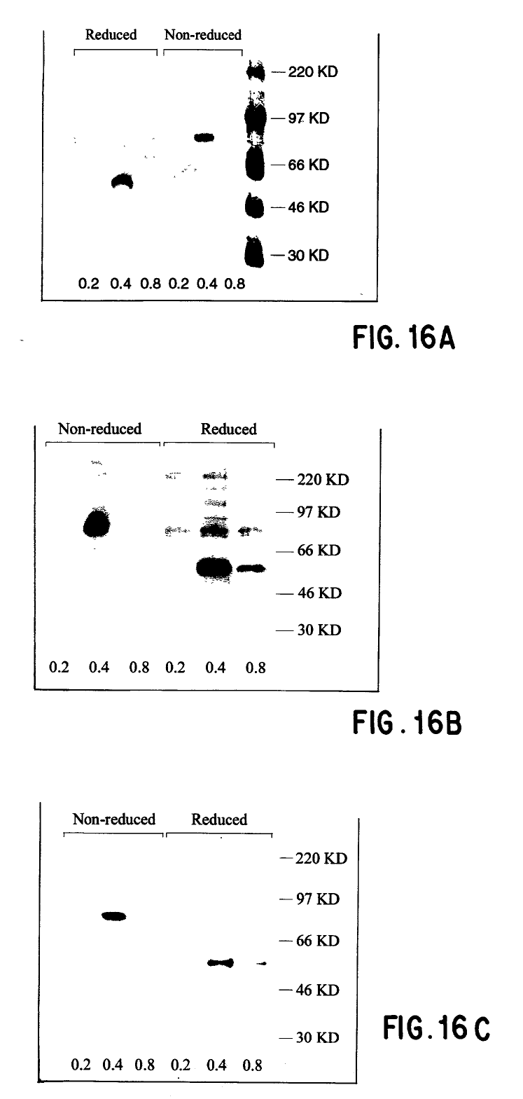

FIGS. 16A-C show the isolation and partial characterization of full length human PDGF-C protein. In FIG. 16A, the recombinant full length protein was visualized on the blot using antipeptide antibodies against the N-terminal peptide(described above). In FIG. 16B, the recombinant full length protein was visualized on the blot using antipeptide antibodies against the internal peptide (described above). The separated proteins were visualized by staining with Coomassie Brilliant Blue (FIG. 16C). The numbers at the bottom of FIGS. 16A-C refer to the concentration of imidazole used to elute the protein from the Ni-NTA column and are expressed in molarity (M). FIGS. 16A-C also show that the full length protein migrates as a 90 kDa species under non-reducing conditions and as a 55 kDa species under reducing conditions. This indicates that the full length protein was expressed as a disulfide-linked dimer.

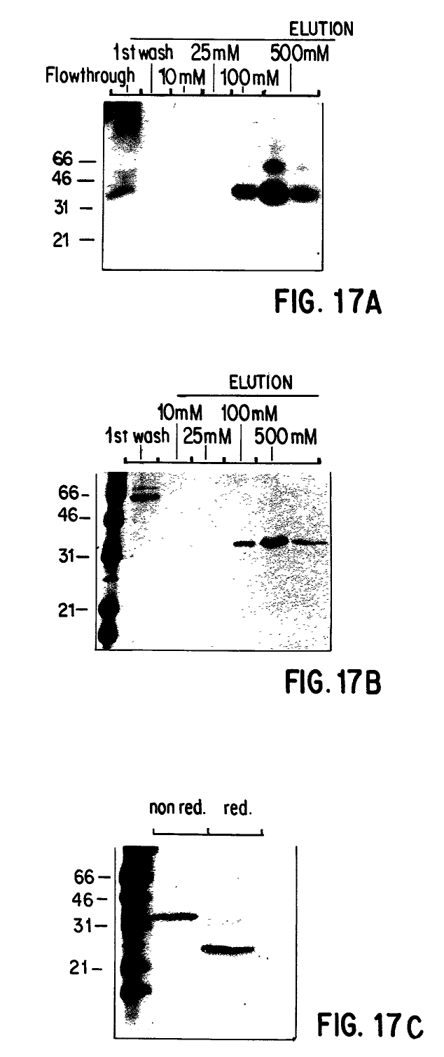

FIGS. 17A-C show the analysis of the isolation and partial characterization of a truncated form of human PDGF-C containing the PDGF/VEGF homology domain only. In FIG. 17A, the immunoblot analysis of fractions eluted from the Ni-agarose column demonstrates that the protein could be eluted at imidazole concentrations ranging between 100-500 mM. The eluted fractions were analyzed under non-reducing conditions, and the truncated human PDGF-C was visualized on the blot using antipeptide antibodies against the internal peptide (described above). FIG. 17B shows the Coomassie Brilliant Blue staining of the same fractions as in FIG. 17A. This shows that the procedure generates highly purified material migrating as a 36 kDa species. FIG. 17C shows the Coomassie Brilliant Blue staining of non-reduced (non-red.) and reduced (red.) truncated human PDGF-C protein. The data show that the protein is a secreted dimer held together by disulfide bonds and that the monomer migrates as a 24 kDa species.

### Example 4

### Receptor Binding Properties of Full Length and Truncated PDGF-C

To assess the interactions between full length and truncated PDGF-C and the VEGF receptors, full length and truncated PDGF-C were tested for their capacity to bind to soluble Ig-fusion proteins containing the extracellular domains of human VEGFR-1, VEGFR-2 and VEGFR-3 (Olofsson et al., Proc. Natl. Acad. Sci. USA, 1998 95 11709-11714). The fusion proteins, designated VEGFR-1-Ig, VEGFR-2-Ig and VEGFR-3-Ig, were transiently expressed in human 293 EBNA cells. All Ig fusion proteins were human VEGFRs. Cells were incubated for 24 hours after transfection, washed with Dulbecco's Modified Eagle Medium (DMEM) containing 0.2% bovine serum albumin and starved for 24 hours. The fusion proteins were then precipitated from the clarified conditioned medium using protein A-Sepharose beads (Pharmacia). The beads were combined with 100 microliters of 10×binding buffer (5% bovine serum albumin, 0.2% Tween 20 and 10 μg/ml heparin) and 900 microliter of conditioned medium from 293 cells that had been transfected with mammalian expression plasmids encoding full length or truncated PDGF-C or control vector, then metabolically labeled with ³⁵S-cysteine and methionine (Promix, Amersham) for 4 to 6 hours. After 2.5 hours, at room temperature, the Sepharose beads were washed 3 times with binding buffer at 4° C., once with phosphate buffered saline and boiled in SDS-PAGE buffer. Labeled proteins that were bound to the Ig-fusion proteins were analyzed by SDS-PAGE under reducing conditions. Radiolabeled proteins were detected using a phosphorimager analyzer. In all these analyses, radiolabeled PDGF-C failed to show any interaction with any of the VEGF receptors.

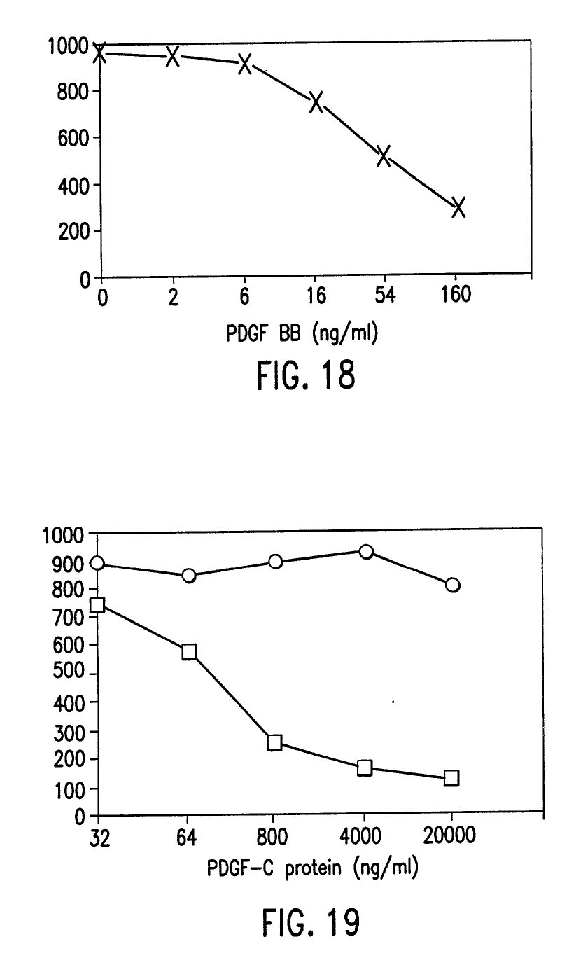

Next, full length and truncated PDGF-C were tested for their capacity to bind to human PDGF receptors alpha and beta by analyzing their abilities to compete with PDGF-BB for binding to PDGF receptors. The binding experiments were performed on porcine aortic endothelial-1 (PAE-1) cells stably expressing the human PDGF receptors alpha and beta (Eriksson et al., EMBO J, 1992, 11, 543-550). Binding experiments were performed essentially as in Heldin et al. (EMBO J, 1988, 7 1387-1393). Different concentrations of human full-length and truncated PDGF-C, or human PDGF-BB were mixed with 5 ng/ml of ¹²⁵I-PDGF-BB in binding buffer (PBS containing 1 mg/ml of bovine serum albumin). Aliquots were incubated with the receptor expressing PAE-1 cells plated in 24-well culture dishes on ice for 90 minutes. After three washes with binding buffer, cell-bound ¹²⁵I-PDGF-BB was extracted by lysis of cells in 20 mM Tris-HCl, pH 7.5, 10% glycerol, 1% Triton X-100. The amount of cell bound radioactivity was determined in a gamma-counter. A standard curve for the binding of ¹²⁵I-labeled PDGF BB homodimers to PAE-1 cells expressing PDGF alpha-receptor is shown in FIG. 18. An increasing excess of the unlabeled protein added to the incubations competed efficiently with cell association of the radiolabeled tracer.

FIG. 19 graphically shows that the truncated PDGF-C efficiently competed for binding to the PDGF alpha-receptor, while the full length protein did not. Both the full length and truncated proteins failed to compete for binding to the PDGF beta-receptor.

### Example 5

### PDGF Alpha-receptor Phosphorylation

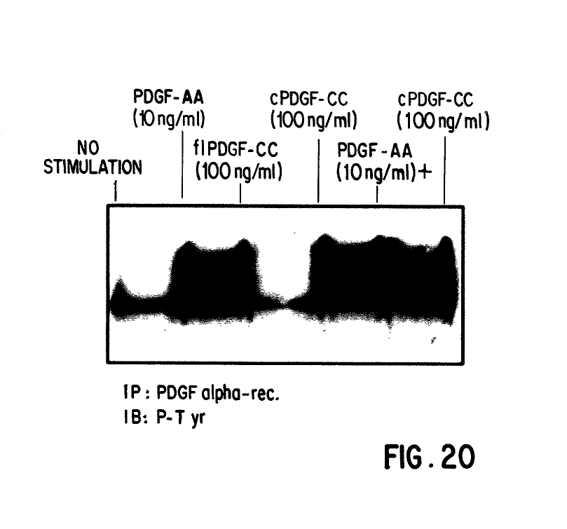

To test if PDGF-C causes increased phosphorylation of the PDGF alpha-receptor, full length and truncated PDGF-C were tested for their capacity to bind to the PDGF alpha-receptor and stimulate increased phosphorylation. Serum-starved porcine aortic endothelial (PAE) cells stably expressing the human PDGF alpha-receptor were incubated on ice for 90 minutes with PBS supplemented with 1 mg/ml BSA and 10 ng/ml of PDGF-AA, 100 ng/ml of full length human PDGF-CC homodimers (flPDGF-CC), 100 ng/ml of truncated PDGF-CC homodimers (cPDGF-CC), or a mixture of 10 ng/ml of PDGF-AA and 100 ng/ml of truncated PDGF-CC. Full length and truncated PDGF-CC homodimers were produced as described above. Sixty minutes after the addition of the polypeptides, the cells were lysed in lysis buffer (20 mM tris-HCl, pH 7.5, 0.5% Triton X-100, 0.5% deoxycholic acid, 10 mM EDTA, 1 mM orthovanadate, 1 mM PMSF 1% Trasylol). The PDGF alpha-receptors were immunoprecipitated from cleared lysates with rabbit antisera against the human PDGF alpha-receptor (Eriksson et al., EMBO J, 1992 11 543-550). The precipitated receptors were applied to a SDS-PAGE gel. After SDS gel electrophoresis, the precipitated receptors were transferred to nitrocellulose filters, and the filters were probed with anti-phosphotyrosine antibody PY-20, (Transduction Laboratories). The filters were then incubated with horseradish peroxidase-conjugated anti-mouse antibodies. Bound antibodies were detected using enhanced chemiluminescence (ECL, Amersham Inc). The filters were then stripped and reprobed with the PDGF alpha-receptor rabbit antisera, and the amount of receptors was determined by incubation with horseradish peroxidase-conjugated anti-rabbit antibodies. Bound antibodies were detected using enhanced chemiluminescence (ECL, Amersham Inc). The probing of the filters with PDGF alpha-receptor antibodies confirmed that equal amounts of the receptor were present in all lanes. PDGF-AA is included in the experiment as a control. FIG. 20 shows that truncated, but not full length PDGF-CC, efficiently induced PDGF alpha-receptor tyrosine phosphorylation. This indicates that truncated PDGF-CC is a potent PDGF alpha-receptor agonist.

### Example 6

### Mitogenicity of PDGF-C for Fibroblasts

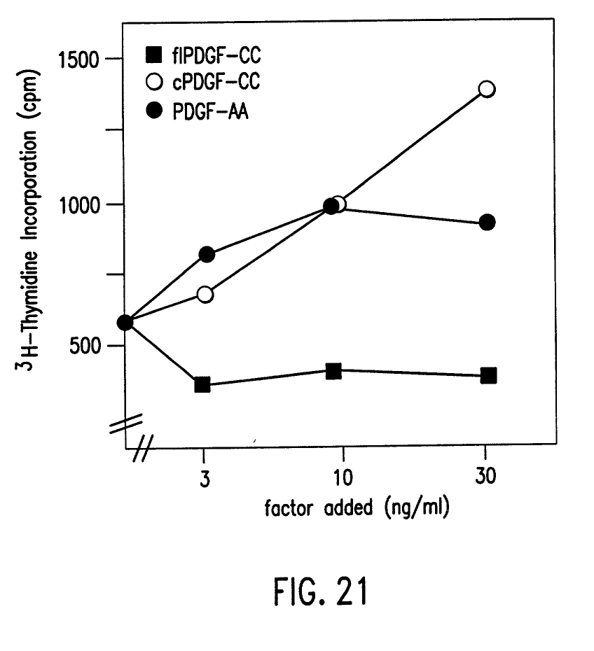

FIG. 21 shows the mitogenic activities of truncated and full length PDGF-CC on fibroblasts. The assay was performed essentially as described in Mori et al., J. Biol. Chem., 1991 266 21158-21164. Serum starved human foreskin fibroblasts were incubated for 24 hours with 1 ml of serum-free medium supplemented with 1 mg/ml BSA and 3 ng/ml, 10 ng/ml or 30 ng/ml of full length PDGF-CC (flPDGF-CC), truncated PDGF-CC (cPDGF-CC) or PDGF-AA in the presence of 0.2 μmCi [3H]thymidine. After trichloroacetic acid (TCA) precipitation, the incorporation of [3H]thymidine into DNA was determined using a beta-counter. The results show that truncated PDGF-CC, but not full length PDGF-CC, is a potent mitogen for fibroblasts. PDGF-AA is included in the experiment as a control.

PDGF-C does not bind to any of the known VEGF receptors. PDGF-C is the only VEGF family member, thus far, which can bind to and increase phosphorylation of the PDGF alpha-receptor. PDGF-C is also the only VEGF family member, thus far, to be a potent mitogen of fibroblasts. These characteristics indicate that the truncated form of PDGF-C may not be a VEGF family member, but instead a novel PDGF. Furthermore, the full length protein is likely to be a latent growth factor that needs to be activated by proteolytic processing to release the active PDGF/VEGF homology domain. A putative proteolytic site is the dibasic motif found in residues 231-234 in the full length protein, residues -R-K-S-R-. This site is structurally conserved in a comparison between mouse and human PDGF-Cs (FIG. 7). Preferred proteases include, but are not limited to, Factor X and enterokinase. The N-terminal CUB domain may be expressed as an inhibitory domain which might be used to localize this latent growth factor in some extracellular compartment (for example the extracellular matrix) and which is removed by limited proteolysis when need, for example during embryonic development, tissue regeneration, tissue remodelling including bone remodelling, active angiogenesis, tumor progression, tumor invasion, metastasis formation and/or wound healing.

### Example 7

### PDGF Receptors Binding of Truncated PDGF-C

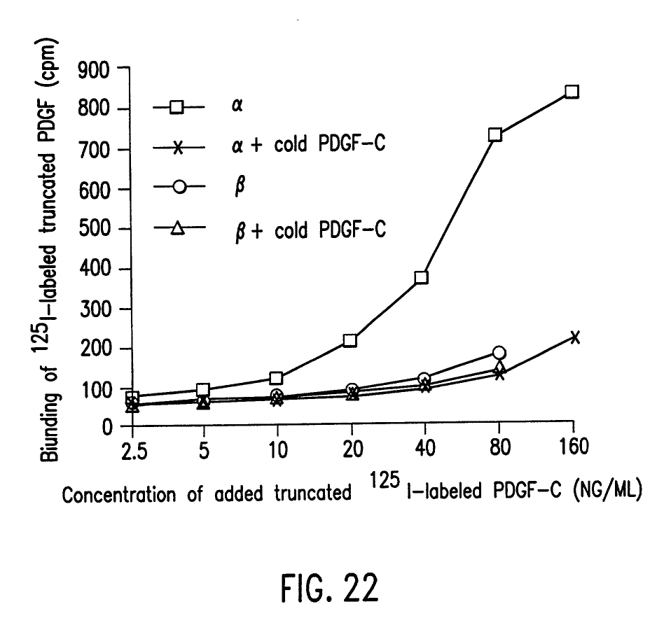

To assess the interactions between truncated PDGF-C and the PDGF alpha and beta receptors, truncated PDGF-C was tested for its capacity to bind to porcine aortic endothelial-1 (PAE-1) cells expressing PDGF alpha or beta receptors, respectively (Eriksson et al., EMBO J, 1992, 11 543-550). The binding experiments were performed essentially as described in Heldin et al. (EMBO J, 1988, 7 1387-1393). Five micrograms of truncated PDGF-C protein in ten microliters of sodium borate buffer was radiolabeled using the Bolton-Hunter reagent (Amersham) to a specific activity of 4×10⁵ cpm/ng. Different concentrations of radiolabeled truncated PDGF-C, with or without added unlabeled protein, in binding buffer (PBS containing 1 mg/ml of bovine serum albumin) was added to the receptor expressing PAE-1 cells plated in 24-well culture dishes on ice for 90 minutes. After three washes with binding buffer, cell-bound ¹²⁵I-labeled PDGF-C was extracted by lysis of cells in 20 mM Tris-HCl, pH 7.5, 10% glycerol, 1% Triton X-100. The amount of cell-bound radioactivity was determined in a gamma-counter. Non-specific binding was estimated by including a 100-fold molar excess of truncated PDGF-C in some experiments. All binding data represents the mean of triplicate analyses and the experimental variation in the experiment varied between 10-15%. As seen in FIG. 22, truncated PDGF-C binds to cells expressing PDGF alpha receptors, but not to beta receptor expressing cells. The binding was specific as radiolabeled PDGF-C was quantitatively displaced by a 100-fold molar excess of unlabeled protein.

### Example 8

### Protease Effects on Full Length PDGF-C

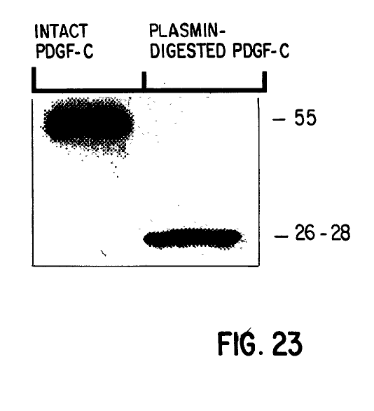

To demonstrate that full length PDGF-C can be activated by limited proteolysis to release the PDGF/VEGF homology domain from the CUB domain, the full length protein was digested with different proteases. For example, full length PDGF-C was digested with plasmin in 20 mM Tris-HCl (pH 7.5) containing 1 mM CaCl₂, 1 mM MgCl₂ and 0.01% Tween 20 for 1.5 to 4.5 hours at 37° C. using two to three units of plasmin (Sigma) per ml. The released domain essentially corresponded in size to the truncated PDGF-C species previously produced in insect cells. Plasmin-digested PDGF-C and undigested full length PDGF-C were applied to a SDS-PAGE gel under reducing conditions. After SDS-PAGE gel electrophoresis, the respective proteins were transferred to a nitrocellulose filter, and the filter was probed using a rabbit antipeptide antiserum to residues 230-250 in full length protein (residues GRKSRVVDLNLLTEEVRLYSC (SEQ ID NO:37) located in just N-terminal to the PDGF/VEGF homology domain). Bound antibodies were detected using enhanced chemiluminescence (ECL, Amersham Inc). FIG. 23 shows the immunoblot with a 55 kDa undigested full length protein and the plasmin-generated 26-28 kDa species.

### Example 9

### PDGF Alpha Receptors Binding of Plasmin-digested PDGF-C

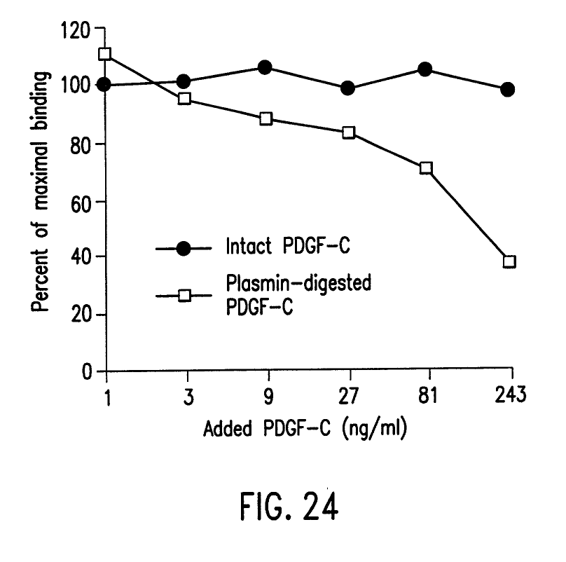

To assess the interactions between plasmin-digested PDGF-C and the PDGF alpha receptors, plasmin-digested PDGF-C was tested for its capacity to bind to porcine aortic endothelial-1 (PAE-1) cells expressing PDGF alpha receptors (Eriksson et al., EMBO J, 1992, 11 543-550). The receptor binding analyses were performed essentially as in Example 7 using 30 ng/ml of ¹²⁵I-labeled truncated PDGF-C as the tracer. As seen in FIG. 24, increasing concentrations of plasmin-digested PDGF-C efficiently competed for binding to the PDGF alpha receptors. In contrast, undigested full length PDGF-C failed to compete for receptor binding. These data indicate that full length PDGF-C is a latent growth factor unable to interact with PDGF alpha receptors and that limited proteolysis, which releases the C-terminal PDGF/VEGF homology domain, is necessary to generate an active PDGF alpha receptor ligand/agonist.

### Example 10

### Cloning and Expression of the Human PDGF-C CUB Domain

A human PDGF-C 430 bp cDNA fragment encoding the CUB domain (amino acid residues 23-159 in full length PDGF-C) was amplified by PCR using Deep Vent DNA polymerase (Biolabs) using standard conditions and procedures. The forward primer used was 5′ cgggatcccgaatccaacctgagtag3′ (SEQ ID NO:38). This primer includes a BamHI site (underlined) for in clone frame cloning. The reverse primer used was 5′ccggaattcctaatggtgatggtgatgatgtttgtcatcgtcgtcgacaatgttgta gtg3′ (SEQ ID NO:39). This primer includes an EcoRI site (underlined) and sequences coding for a C-terminal 6×His tag preceded by an enterokinase site. The amplified PCR fragment was subsequently cloned into a pACgp67A transfer vector. Verification of the correct sequence of the expression construct, CUB-pACgp67A, was by automatic nucleotide sequencing. The expression vectors were then co-transfected with BaculoGold linearized baculovirus DNA into Sf9 insect cells according to the manufacture's protocol (Pharmingen). Recombined baculovirus were amplified several times before beginning large scale protein production and protein purification according to the manual (Pharmingen).

Sf9 cells, adapted to serum free medium, were infected with recombinant baculovirus at a multiplicity of infection of about 7. Media containing the recombinant proteins were harvested 72 hours after infection and were incubated with Ni-NTA-Agarose beads (Qiagen) overnight at 4° C. The beads were collected in a column and after extensive washing with 50 mM sodium phosphate buffer pH 8, containing 300 mM NaCl (the washing buffer), the bound proteins were eluted with increasing concentrations of imidazole (from 100 mM to 400 mM) in the washing buffer. The eluted proteins were analyzed by SDS-PAGE using a polyacrylamide gel under reducing and non-reducing conditions.

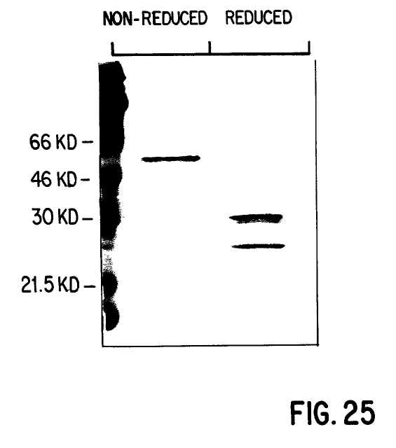

FIG. 25 shows the results from Coomassie blue staining of the gel. The human PDGF-C CUB domain is a disulfide-linked homodimer with a molecular weight of about 55 KD under non-reducing conditions, while two monomers of about 25 and 30 KD respectively are present under reducing conditions. The heterogeneity is probably due to heterogenous glycosylation of the two putative N-linked glycosylation sites present in the CUB domain at amino acid positions 25 and 55. A protein marker lane is shown to the left in the figure.

### Example 11

### Localization of PDGF-C Transcripts in Developing Mouse Embryos

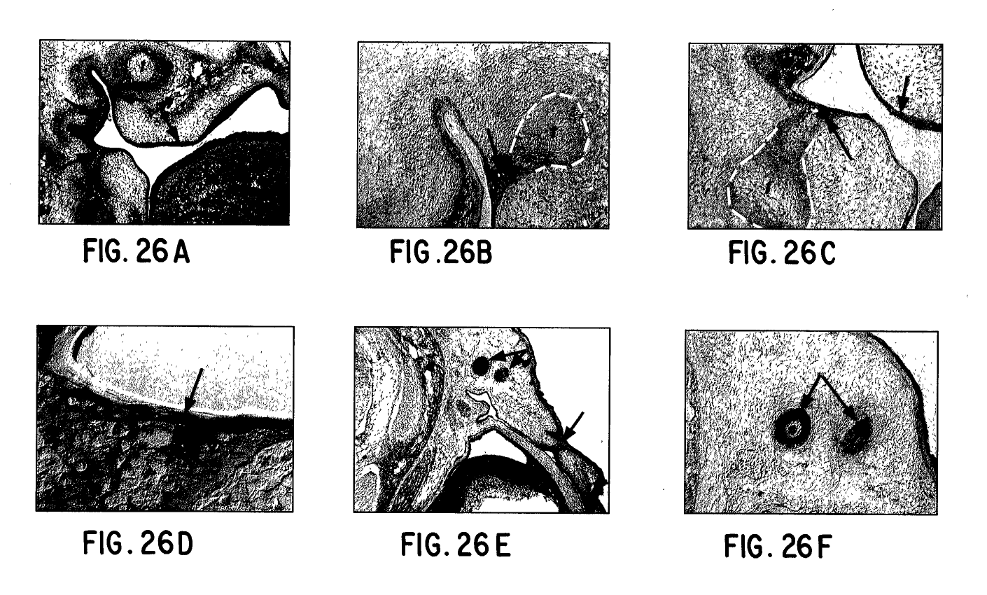

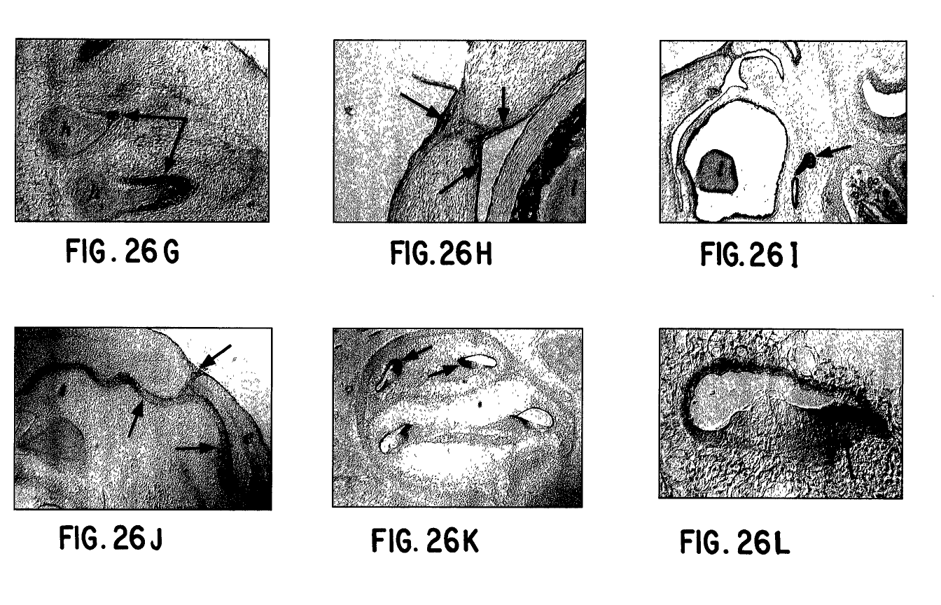

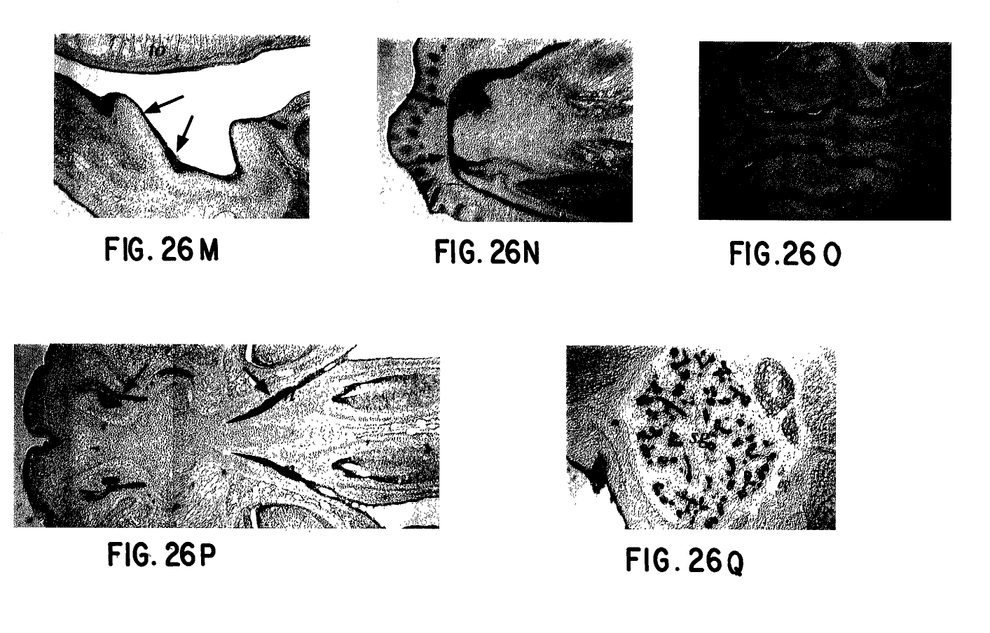

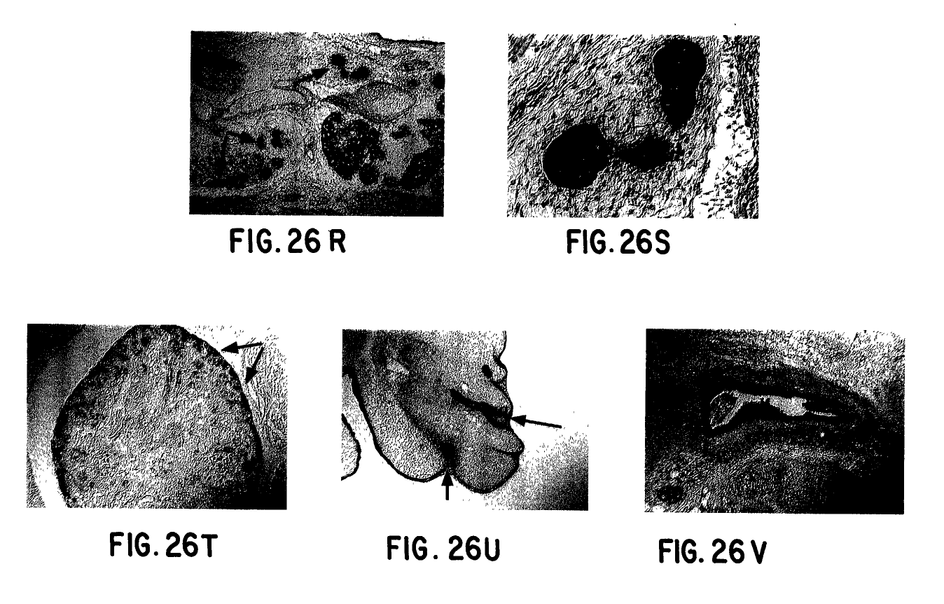

To gain insight into the biological function of PDGF-C, PDGF-C expression in mouse embryos was localized by non-radioactive in situ hybridization in tissue sections from the head (FIGS. 26A-26S) and urogenital tract (FIGS. 26T-26V) regions. The non-radioactive in situ hybridization employed protocols and PDGF-A and PDGFR-alpha probes are described in Boström et al., Cell, 1996 85 863-873, which is hereby incorporated by reference. The PDGF-C probe was derived from a mouse PDGF-C cDNA. The hybridization patterns shown in FIGS. 26A-26V are for embryos aged E16.5, but analogous patterns are seen at E14.5, E15.5 and E17.5. Sense probes were used as controls and gave no consistent pattern of hybridization to the sections.

FIG. 26A shows the frontal section through the mouth cavity at the level of the tooth anlagen (t). The arrows point to sites of PDGF-C expression in the oral ectoderm. Also shown is the tongue (to). FIGS. 26B-26D show PDGF-C expression in epithelial cells of the developing tooth canal. Individual cells are strongly labeled in this area (arrow in FIG. 26D), as well as in the developing palate ectoderm (right arrow in FIG. 26C). FIG. 26E shows the frontal section through the eye, where PDGF-C expression is seen in the hair follicles (double arrow) and in the developing eyelid. Also shown is the retina (r). In FIGS. 26F and 26G, the PDGF-C expression is found in the outer root sheath of the developing hair follicle epithelium. In FIG. 26H, PDGF-C expression is shown in the developing eyelid. There is an occurrence of individual strongly PDGF-C positive cells in the developing opening. Also shown is the lens (1). In FIG. 26I, PDGF-C expression in the developing lacrimal gland is shown by the arrow. In FIG. 26J, PDGF-C expression in the developing external ear is shown. Expression is seen in the external auditory meatus (left arrow) and in the epidermal cleft separating the prospective auricle (e). FIGS. 26K and 26L show PDGF-C expression in the cochlea. Expression is seen in the semi-circular canals (arrows in 26K). There is a polarized distribution of PDGF-C mRNA in epithelial cells adjacent to the developing hair cells (arrow in 26L). FIGS. 26M and 26N show PDGF-C expression in the oral cavity. Horizontal sections show expression in buccal epithelium (arrows in 26M) and in the forming cleft between the lower lip buccal and the gingival epithelium (arrows in 26N). Also shown is the tooth anlagen (t) and the tongue (to). FIGS. 26O and 26P show PDGF-C expression in the developing nostrils, shown on horizontal sections. PDGF-C expression appears strongest before stratification of the epithelium and the formation of the canal proper (arrows in 26O and 26P). Also shown is the developing nostrils (n). FIGS. 26Q-26S show PDGF-C expression in developing salivary glands and ducts. FIG. 26Q is the sublingual gland. FIGS. 26R and 26S show the maxillary glands, the salivary gland (sg) and the salivary duct (sd). FIGS. 26T-26V show the expression of PDGF-C in the urogenital tract. FIG. 26T shows the expression of PDGF-C in the developing kidney metanephric mesoderm. FIG. 26U shows the expression of PDGF-C in the urethra (ua) and in epithelium surrounding the developing penis. FIG. 26V shows the PDGF-C expression in the developing ureter (u).

### Example 12

### PDGF-C, PDGF-A and PDGFR-alpha Expression in the Developing Kidney

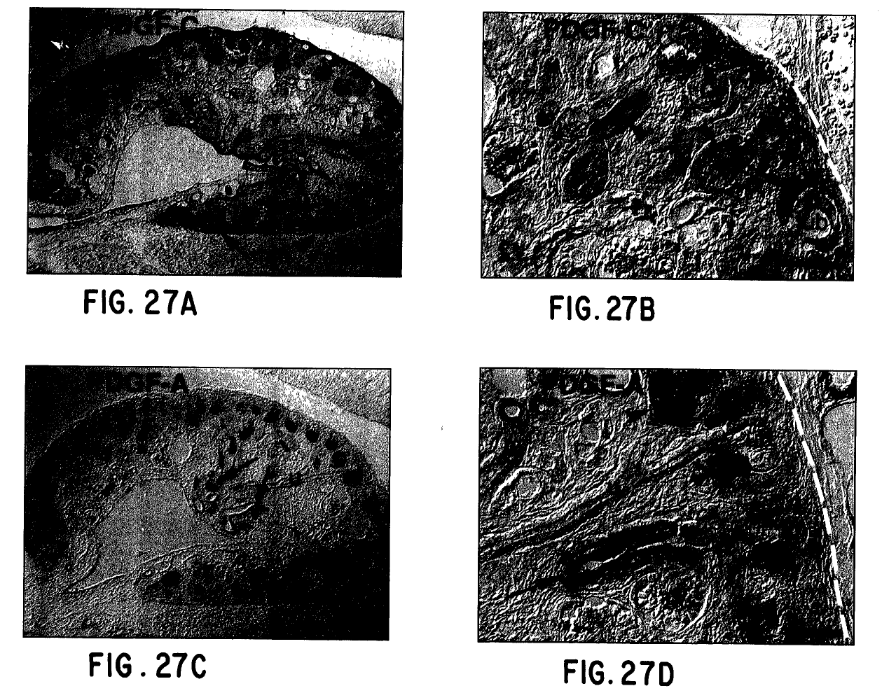

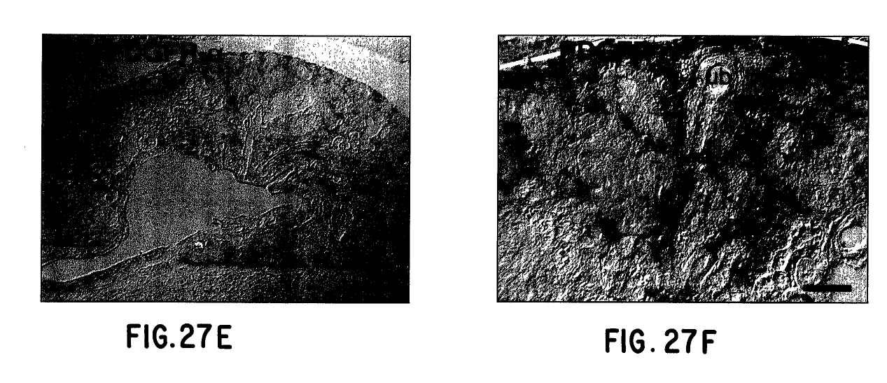

One of the strongest sites of PDGF-C expression is the developing kidney and so expression of PDGF-C, PDGF-A and PDGFR-alpha was looked at in the developing kidney. FIGS. 27A-27F show the results of non-radioactive in situ hybridization demonstrating the expression (blue staining in unstained background visualized using DIC optics) of mRNA for PDGF-C (FIGS. 27A and 27B), PDGF-A (FIGS. 27C and 27D) and PDGFR-alpha (FIGS. 27E and 27F) in E16.5 kidneys. The white hatched line in FIGS. 27B, 27D and 27F outlines the cortex border. The bar in FIGS. 27A, 27C and 27E represents 250 μm, and in FIGS. 27B, 27D and 27F represents 50 μm.

PDGF-C expression is seen in the metanephric mesenchyme (mm in FIG. 27A), and appears to be upregulated in the condensed mesenchyme (arrows in FIG. 27B) undergoing epithelial conversion as a prelude to tubular development, which is situated on each side of the ureter bud (ub). PDGF-C expression remains at lower levels in the early nephronal epithelial aggregates (arrowheads in B), but is absent from mature glomeruli (gl) and tubular structures.

PDGF-A expression is not seen in these early aggregates, but is strong in later stages of tubular development (FIGS. 24C and 24D). PDGF-A is expressed in early nephronal epithelial aggregates (arrowheads in FIG. 27D), but once the nephron is developed further, PDGF-A expression becomes restricted to the developing Henle's loop (arrow in FIG. 27D). The strongest expression is seen in the Henle's loops in the developing marrow (arrows in FIG. 27C). The branching ureter (u) and the ureter bud (ub) is negative for PDGF-A.

Thus, the PDGF-C and PDGF-A expression patterns in the developing nephron are spatially and temporally distinct. PDGF-C is expressed in the earliest stages (mesenchymal aggregates) and PDGF-A in the latest stages (Henle's loop formation) of nephron development.

PDGFR-alpha is expressed throughout the mesenchyme of the developing kidney (FIGS. 27E and 27F) and may hence be targeted by both PDGF-C and PDGF-A. PDGF-B expression is also seen in the developing kidney, but occurs only in vascular endothelial cells. PDGFR-beta expression takes place in perivascular mesenchyme, and its activation by PDGF-B is critical for mesangial cell recruitment into glomeruli.

These results demonstrate that PDGF-C expression occurs in close spatial relationship to sites of PDGFR-alpha expression, and are distinct from the expression sites of PDGF-A or PDGF-B. This indicates that PDGF-C may act through PDGFR-alpha in vivo, and may have functions that are not shared with PDGF-A and PDGF-B.

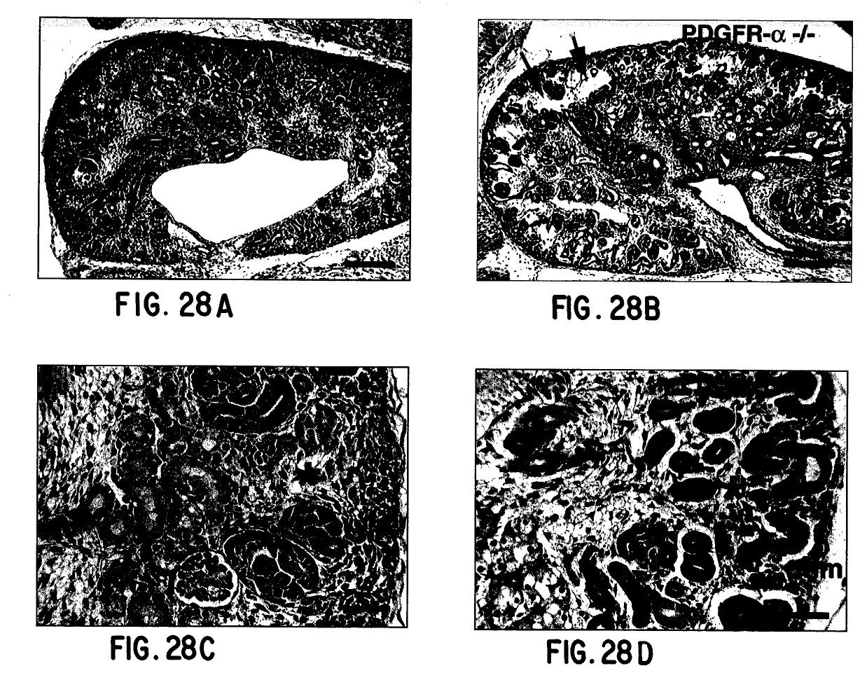

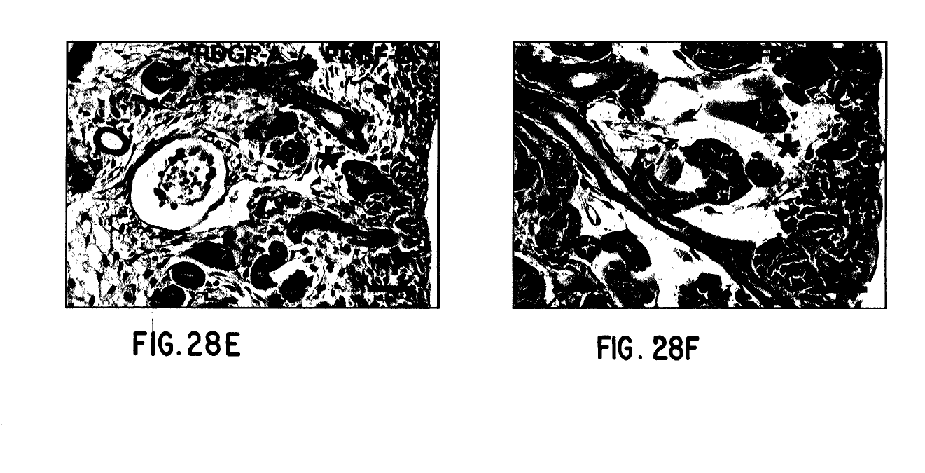

Since the unique expression pattern of PDGF-C in the developing kidney indicates a function as a PDGFR-alpha agonist separate from that of PDGF-A or -B, a comparison was made to the histology of embryonic day 16.5 kidneys from PDGFR-alpha knockout mice (FIGS. 28B and 28F) with kidneys from wildtype (FIGS. 28A and 28C), PDGF-A knockout (FIG. 28D) and PDGF-A/PDGF-B double knockout (FIG. 28E) mice. The bar in FIGS. 28A and 28B represents 250 mm, and in FIGS. 28C-28F represents 50 μm.

Heterozygote mutants of PDGF-A, PDGF-B and PDGFR-alpha (Boström et al., Cell, 1996 85 863-873; Levéen et al., Genes Dev., 1994 8 1875-1887; Soriano et al., Development, 1997 124 2691-70) were bred as C57Bl6/129sv hybrids and intercrossed to produce homozygous mutant embryos. PDGF-A/PDGF-B heterozygote mutants were crossed to generate double PDGF-A/PDGF-B knockout embryos. Due to a high degree of lethality of PDGF-A −/−embryos before E10 (Boström et al., Cell, 1996 85 863-873), the proportion of double knockout E16.5 embryos obtained in such crosses were less than 1/40. The histology of kidney phenotypes was verified on at least two embryos of each genotype, except the PDGF-A/PDGF-B double knockout for which only a single embryo was obtained.

It is interesting that there is lack of interstitial mesenchyme in the cortex of PDGFR-alpha −/−kidney (arrows in FIG. 28A and asterisk in FIG. 28F) and the presence of interstitial mesenchyme in all other genotypes (asterisks in FIG. 28C-E). The branching ureter (u) and the metanephric mesenchyme (mm) and its epithelial derivatives appear normal in all mutants. The abnormal glomerulus in the PDGF-A/PDGF-B double knockout reflect failure of mesangial cell recruitment into the glomerular tuft due to the absence of PDGF-B.

These results indicate that PDGFR-alpha knockouts have a kidney phenotype, which is not seen in PDGF-A or PDGF-A/PDGF-B knockouts, hence potentially reflecting loss of signaling by PDGF-C. The phenotype consists of the marked loss of interstitial mesenchyme in the developing kidney cortex. The cells lost in PDGFR-alpha −/−kidneys are thus normally PDGFR-alpha positive cells adjacent to the site of expression of PDGF-C.

### Example 13

### Chick Embryo Chorioallantoic Membrane (CAM) Assay for Angiogenic Activity

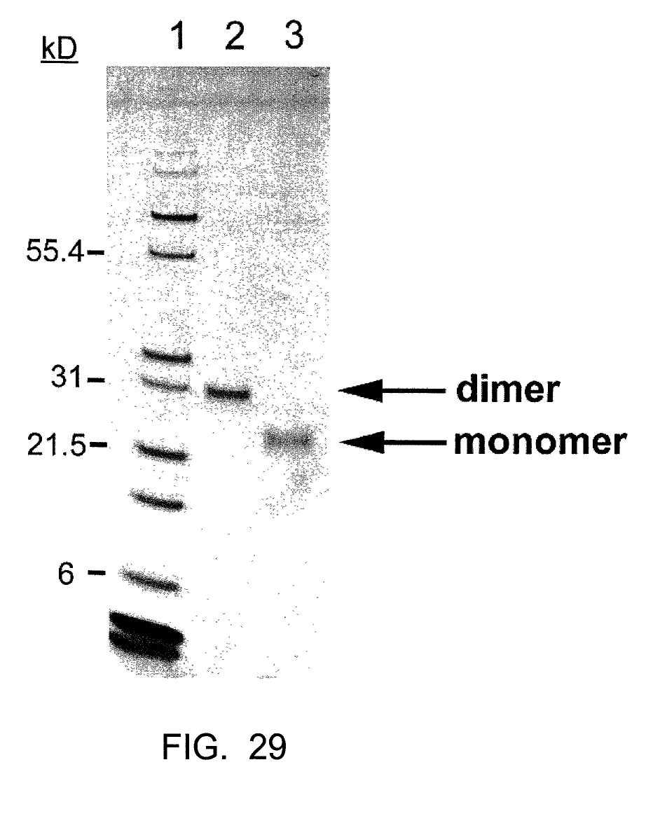

Recombinant human PDGF-CC core domain protein was expressed as described above (Cf. Li et al., Nat Cell Biol 2000 2 302-9) and purified to homogeneity. Two micrograms of the purified PDGF-CC were analyzed on a 4-12% gradient BisTris NUPAGE (Norex) polyacrylamide gel followed by staining with Coomassie Blue. The results are shown in FIG. 29. Dimeric (lane 2) and monomeric (lane 3) forms of PDGF-CC were detected under non-reducing and reducing/alkylating conditions, respectively. Molecular mass markers are indicated on the left (lane 1). Under non-reducing conditions the core domain of PDGF-CC appeared as dimers with the expected molecular mass of 31 kDa (lane 2). The dimeric forms of PDGF-CC were converted to monomers under reducing conditions in the presence of DTT (lane 3).

The chick embryo chorioallantoic membrane (CAM) assay was performed according to previously published methods (Cao et al., Proc Natl Acad Sci USA 1998 95 14389-94; Cao et al., Proc Natl Acad Sci USA 1999 96 5728-33). Three-day-old fertilized white Leghorn eggs (OVA Production, Sorgarden, Sweden) were cracked, and chick embryos with intact yolks were carefully placed in 20×100 mm plastic petri-dishes. After 6 days of incubation in 3% CO₂ at 37° C., a disk of methylcellulose containing 2.5 μg of truncated PCGF-C homodimer (PDGF-CC) or BSA alone dried on a nylon mesh (3×3 mm) was implanted on the CAM of individual embryos. The nylon mesh disks were made by desiccation of 10 gl of 0.45% methylcellulose in H₂O. After 4-5 days of incubation, embryos and CAMs were examined for the formation of new blood vessels in the field of the implanted disks using a stereoscope. Disks of methylcellulose containing 2.5 μg of BSA were used as negative controls. The experiments were carried out three times, and 9 embryos/sample were used for each experiment.

The CAM assay, which detects angiogenic activity of compounds during embryonic development, is one of the most widely used in vivo angiogenesis assays (Jain et al., Nat Med 1997 3 1203-8). The early embryos in this angiogenesis assay avoid immune reactions and inflammatory influences on growing vessels. To demonstrate that PDGF-CC could induce angiogenesis in vivo, the core domain of PDGF-CC protein was implanted onto the chick chorioallantoic membrane in the developing embryo.

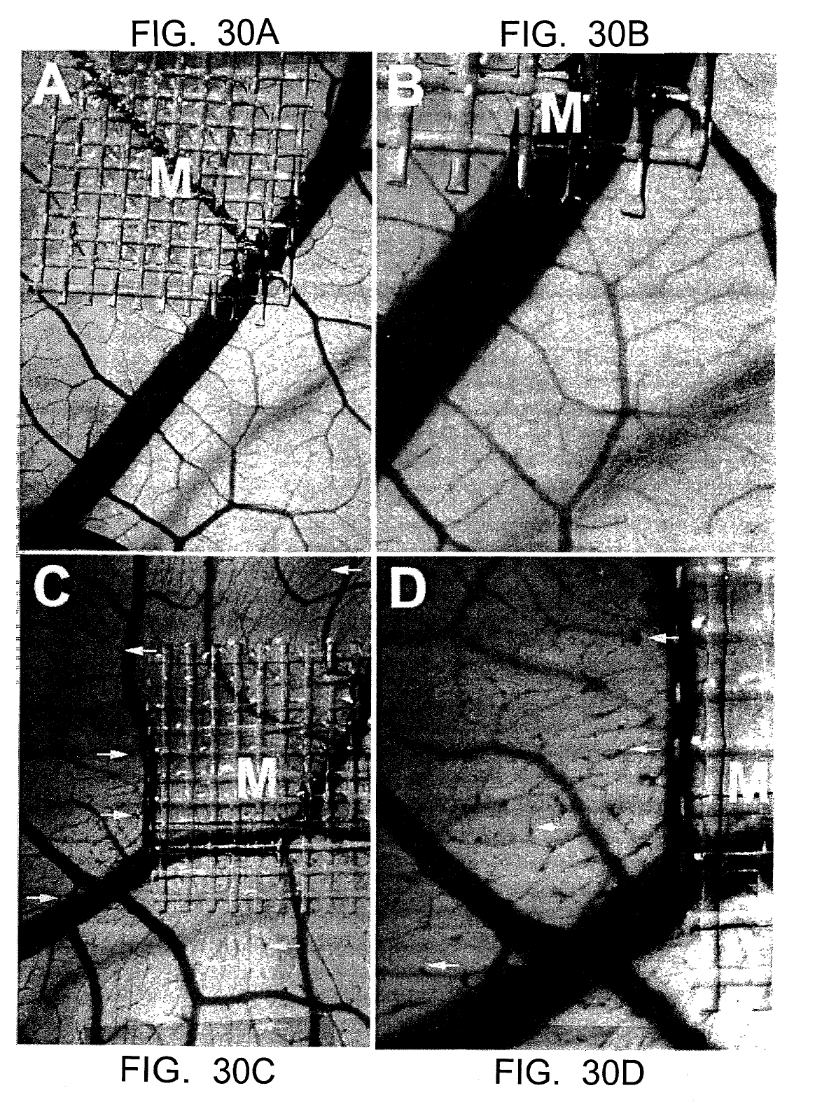

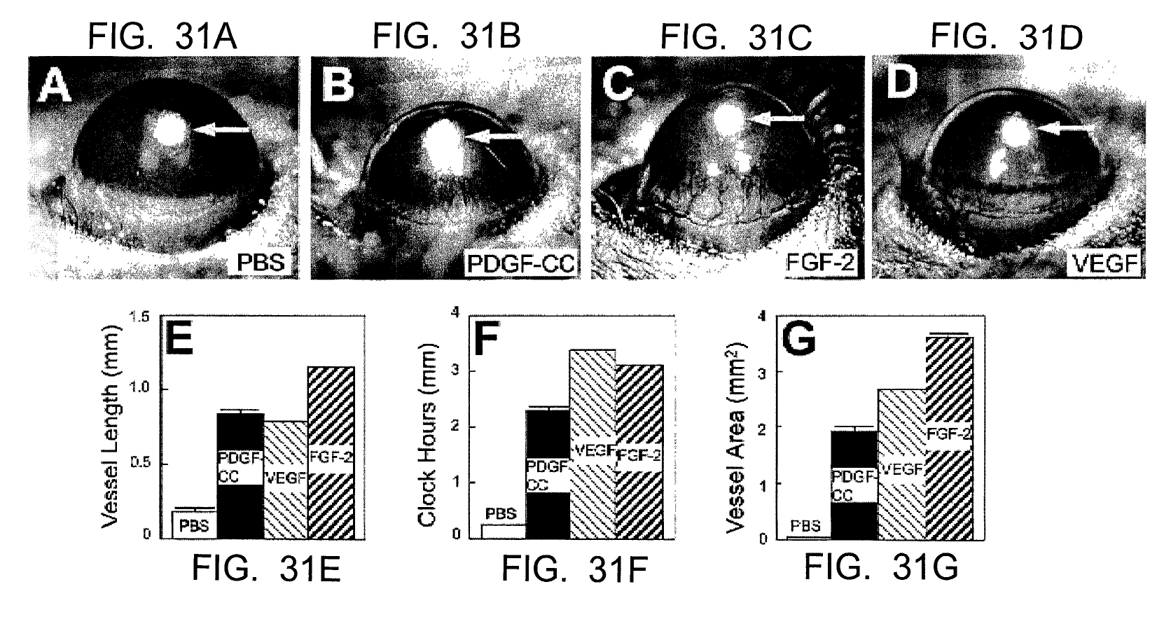

Nylon meshes (9 mm²) coated with 0.45% methylcellulose containing 2.5 μg of PDGF-CC or BSA were implanted on CAMs of 6-day-old chick embryos. After 5 days of implantation, the formation of new blood vessels was examined under a stereoscope. FIGS. 30A, 30B show a CAM with a methylcellulose mesh containing BSA alone, which served as a negative control. FIGS. 30C, 30D show an example of 2.5 μg of PDGF-CC-implanted CAM. New blood vessels and sprouts are marked with arrows in FIGS. 30C and 30D.

It can be seen that PDGF-CC at the dose of 2.5 μg/disk was able to stimulate microvessel growth in each implanted chick embryo. A significant increase of neovascularization with a high vessel density was observed in the surrounding areas of PDGF-CC implant. Notably, PDGF-CC induced the formation of new branches and induced vessel sprouts (small arrows in FIGS. 30C and 30D) from the existing vessels that grew toward the implanted disks. These vessel sprouts appeared as “red dots” budding from blood vessels adjacent to the implanted factors. In contrast, disks without growth factors did not seem to stimulate neovascularization in chick embryos (FIGS. 30A, 30B).

The results clearly demonstrate that the truncated PDGF-C homodimer exhibits marked angiogenic activity in vivo.

### Example 14

### Mouse Corneal Micropocket Assay for Angiogenic Activity

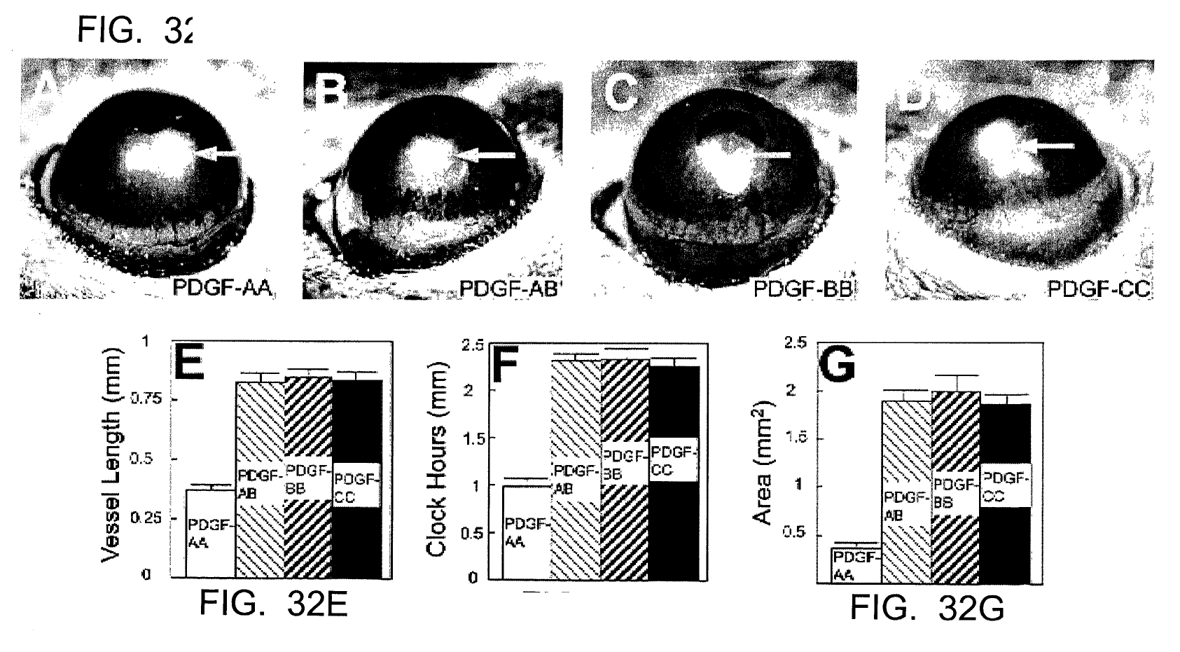

The mouse corneal micropocket assay was performed according to procedures previously described (Cao et al., Proc Natl Acad Sci USA 1998 95 14389-94; Cao et al., Nature 1999 398 381). Male 5-6 week-old C57BI6/J mice were acclimated and caged in groups of six or less. Animals were anaesthetized by injection of a mixture of dormicum and hypnorm (1:1) before all procedures. Corneal micropockets were created with a modified von Graefe cataract knife in both eyes of each male 5-6-week-old C57BI6/J mouse. A micropellet (0.35×0.35 mm) of sucrose aluminum sulfate (Bukh Meditec, Copenhagen, Denmark) coated with slow-release hydron polymer type NCC (IFN Sciences, New Brunswick, N.J.) containing various amounts of truncated PDGF-C homodimer (PDGF-CC) was surgically implanted into each cornal pocket. For comparison purposes corresponding amounts of PDGF-AA, PDGF-AB, PDGF-BB, VEGF₁₆₅ (all obtained commercially from R&D Systems of Minnepolis, Minn.) or FGF-2 (Pharmacia & Upjohn, Milan, Italy) were similarly implanted into corneal pockets of test mice. In each case, the pellet was positioned 0.6-0.8 mm from the corneal limbus. After implantation, erythromycin/ophthalmic ointment was applied to each eye. On day 5 after growth factor implantation, animals were sacrificed with a lethal dose of CO₂, and corneal neovascularization was measured and photographed with a slit-lamp stereomicroscope. In FIGS. 31 and 32, arrows point to the implanted pellets. The photographs represent 20×amplification of the mouse eye. Vessel length and clock hours of circumferential neovascularization were measured. Quantitation of corneal neovascularization is presented as maximal vessel length (FIG. 31E), clock hours of circumferential neovascnlarization (FIG. 31F), and area of neovascularization (FIG. 31G). Graphs represent mean values (±SEM) of 11-16 eyes (6-8 mice) in each group.

The corneal angiogenesis model is one of the most rigorous mammalian angiogenesis models that requires a putative compound to be sufficiently potent in order to induce neovascularization in the corneal avascular tissue. Potent angiogenic factors including FGF-2 and VEGF have profound effects in this system.

The angiogenic response of corneas stimulated by 160 ng of PDGF-CC was robust with a high number of capillaries (FIG. 31B). The newly formed as well as the limbal vessels were markedly dilated in the PDGF-CC-implanted corneas. The capillary vessel length of about 0.8 mm in corneas implanted with PDGF-CC was similar to that found in VEGF-induced vessels (FIGS. 31B, 31D and 31E).

The overall angiogenic response induced by PDGF-CC (FIG. 31B) was similar to that induced by FGF-2 (FIG. 31C), albeit less potent than FGF-2. Both PDGF-CC- and FGF-2-induced microvessels were well organized and separated (FIGS. 31B and 31C). In contrast, the VEGF-induced blood vessels (FIG. 31D) seemed to be leaky, hemorrhagic and likely to rupture. At the front edge, the VEGF-induced capillaries were fused to into disorganized and sinusoidal structures. Thus, angiogenic responses induced by PDGF-CC and VEGF are markedly different from those induced by VEGF but similar to those induced by FGF-2.

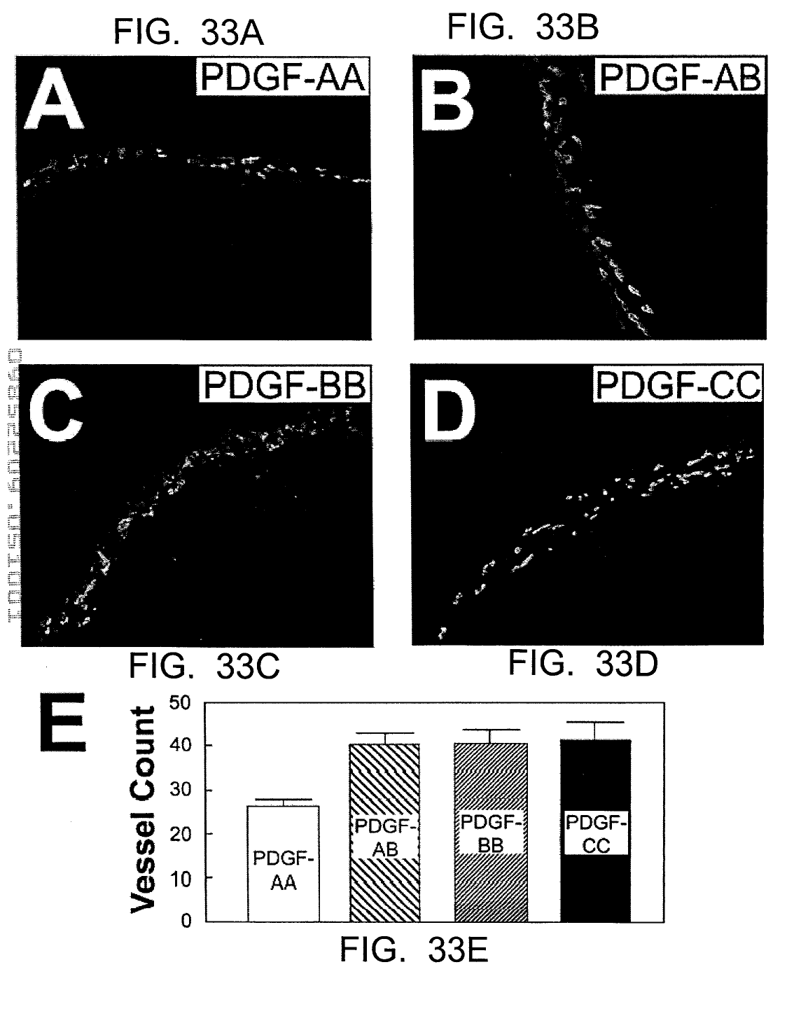

The growth factor-implanted mouse eyes were enucleated at day 6 after implantation and immediately frozen on dry ice and stored at −80° C. before use. Tissue sections of 12 gm were dissected by a cryostat and were immersed in acetone for 10 min. Tissue slices were washed with PBS, blocked with 30% rabbit serum in PBS for 20 min. and incubated for 1 hour with a monoclonal rat anti-mouse antibody against CD31 antigen (PharMingen). After washing with PBS, a secondary FITC-conjugated rabbit anti-rat IgG was incubated with the tissue sections for 1 hour. The immuno-stained signals were examined under a fluorescence microscope. Corneal microvessels were counted in at least 6 sections at 20×magnification. FIGS. 33A-D show histological sections of PDGF-AA (FIG. 33A), PDGF-AB (FIG. 33B), PDGF-BB (FIG. 33C) and PDGF-CC (FIG. 33D) implanted corneas which were incubated with an anti-CD31 antibody and stained with a FITC-conjugated secondary antibody. Microvessels are present in all sections. Vessel counts (FIG. 33E) per 20×field are presented as mean determinants (±SEM) of 6-8 serial sections in each group.

The results again clearly demonstrate that the truncated PDGF-C homodimer exhibits marked angiogenic activity in vivo.

As can be seen in FIG. 32D, truncated PDGF-C homodimer (PDGF-CC) is able to induce angiogenesis in the mouse cornea similar to other dimeric isoforms of PDGFs including PDGF-AA, PDGF-AB, and PDGF-BB. Homodimers of PDGF-BB (FIG. 32C) and PDGF-CC (FIG. 32D), and the heterodimer PDGF-AB (FIG. 32B) induced a similar angiogenic pattern in the mouse cornea. The measured vessel length (FIG. 32E), clock hours (FIG. 32F), and area of neovascularization (FIG. 32G) stimulated by the same amount of these three isoforms were indistinguishable from each other. Consistent with the area of vascularization, the immunohistological studies with the anti-CD31 antibody revealed that microvessel densities induced by PDGF-AB, PDGF-BB and PDGF-CC were virtually identical (FIG. 33B-E). In contrast, the vessel length (FIG. 32E) vessel clock hours (FIG. 32F), vascular area (FIG. 32G) and vessel density (FIGS. 33A and 33E) stimulated by PDGF-AA were significantly less than those induced by PDGF-AB, PDGF-BB or PDGF-CC (FIGS. 32 and 33). All four isoforms of the PDGFs stimulated blood vessels that were dilated (FIGS. 32A-32D).

The test results show that although PDGF-AA also induces angiogenesis in vivo, it does so to a lesser extent than PDGF-CC. It also has been shown that PDGF-AA lacks the ability to directly induce endothelial cell proliferation, migration, and tube formation in vitro (Smits et al., Growth Factors 1989 2 1-8); Marx et al., J Clin Invest 1994 93 131-9); Koyama et al., J Cell Physiol 1994 158 1-6); Sato et al., Am J Pathol 1993 142 1119-30); Plate et al., Lab Invest 1992 67 529-34). Because PDGF-CC, like PDGF-AA, only activates the PDGFR-A receptor, the different angiogenic activity of PDGF-CC in vivo must be regarded as unexpected.

In light of the foregoing test results, which demonstrate the in vivo angiogenesis inducing activity of PDGF-CC, treatments with PDGF-CC alone, or in combination with other angiogenic factors such as VEGF and FGF-2, provides an attractive approach for therapeutic angiogenesis of ischemic heart and limb disorders.

### Bioassays to Determine the Function of PDGF-C

Assays are conducted to evaluate whether PDGF-C has similar activities to PDGF-A, PDGF-B, VEGF, VEGF-B, VEGF-C and/or VEGF-D in relation to growth and/or motility of connective tissue cells, fibroblasts, myofibroblasts and glial cells; to endothelial cell function; to angiogenesis; and to wound healing. Further assays may also be performed, depending on the results of receptor binding distribution studies.

I. Mitogenicity of PDGF-C for Endothelial Cells

To test the mitogenic capacity of PDGF-C for endothelial cells, the PDGF-C polypeptide is introduced into cell culture medium containing 5% serum and applied to bovine aortic endothelial cells (BAEs) propagated in medium containing 10% serum. The BAEs are previously seeded in 24-well dishes at a density of 10,000 cells per well the day before addition of the PDGF-C. Three days after addition of this polypeptide the cells were dissociated with trypsin and counted. Purified VEGF is included in the experiment as positive control.

II. Assays of Endothelial Cell Function

a) Endothelial cell proliferation

Endothelial cell growth assays are performed by methods well known in the art, e.g. those of Ferrara & Henzel, Nature, 1989 380 439-443, Gospodarowicz et al., Proc. Natl. Acad. Sci. USA, 1989 86 7311-7315, and/or Claffey et al., Biochem. Biophys. Acta, 1995 1246 1-9.

b) Cell adhesion assay

The effect of PDGF-C on adhesion of polymorphonuclear granulocytes to endothelial cells is tested.

c) Chemotaxis

The standard Boyden chamber chemotaxis assay is used to test the effect of PDGF-C on chemotaxis.

d) Plasminogen activator assay

Endothelial cells are tested for the effect of PDGF-C on plasminogen activator and plasminogen activator inhibitor production, using the method of Pepper et al., Biochem. Biophys. Res. Commun., 1991 181 902-906.

e) Endothelial cell Migration assay

The ability of PDGF-C to stimulate endothelial cells to migrate and form tubes is assayed as described in Montesano et al., Proc. Natl. Acad. Sci. USA, 1986 83 7297-7301. Alternatively, the three-dimensional collagen gel assay described in Joukov et al., EMBO J., 1996 15 290-298 or a gelatinized membrane in a modified Boyden chamber (Glaser et al., Nature, 1980 288 483-484) may be used.

III. Angiogenesis Assay

The ability of PDGF-C to induce an angiogenic response in chick chorioallantoic membrane is tested as described in Leung et al., Science, 1989 246 1306-1309. Alternatively the rat cornea assay of Rastinejad et al., Cell, 1989 56 345-355 may be used; this is an accepted method for assay of in vivo angiogenesis, and the results are readily transferrable to other in vivo systems.

IV. Wound Healing

The ability of PDGF-C to stimulate wound healing is tested in the most clinically relevant model available, as described in Schilling et al., Surgery, 1959 46 702-710 and utilized by Hunt et al., Surgery, 1967 114 302-307.

V. The Haemopoietic System

A variety of in vitro and in vivo assays using specific cell populations of the haemopoietic system are known in the art, and are outlined below. In particular a variety of in vitro murine stem cell assays using fluorescence-activated cell sorter to purified cells are particularly convenient:

a) Repopulating Stem Cells

These are cells capable of repopulating the bone marrow of lethally irradiated mice, and have the Lin⁻, Rh^{h1}, Ly-6A/E⁺, c-kit⁺ phenotype. PDGF-C is tested on these cells either alone, or by co-incubation with other factors, followed by measurement of cellular proliferation by ³H-thymidine incorporation.

b) Late Stage Stem Cells

These are cells that have comparatively little bone marrow repopulating ability, but can generate D13 CFU-S. These cells have the Lin⁻, Rh^{h1}, Ly-6A/E⁺, c-kit⁺ phenotype. PDGF-C is incubated with these cells for a period of time, injected into lethally irradiated recipients, and the number of D13 spleen colonies enumerated.

c) Progenitor-Enriched Cells

These are cells that respond in vitro to single growth factors and have the Lin⁻, Rh^{h1}, Ly-6A/E⁺, c-kit⁺ phenotype. This assay will show if PDGF-C can act directly on haemopoietic progenitor cells. PDGF-C is incubated with these cells in agar cultures, and the number of colonies present after 7-14 days is counted.

VI. Atherosclerosis

Smooth muscle cells play a crucial role in the development or initiation of atherosclerosis, requiring a change of their phenotype from a contractile to a synthetic state. Macrophages, endothelial cells, T lymphocytes and platelets all play a role in the development of atherosclerotic plaques by influencing the growth and phenotypic modulations of smooth muscle cell. An in vitro assay using a modified Rose chamber in which different cell types are seeded on to opposite cover slips measures the proliferative rate and phenotypic modulations of smooth muscle cells in a multicellular environment, and is used to assess the effect of PDGF-C on smooth muscle cells.

VII. Metastasis

The ability of PDGF-C to inhibit metastasis is assayed using the Lewis lung carcinoma model, for example using the method of Cao et al., J. Exp. Med., 1995 182 2069-2077.

VIII. Migration of Smooth Muscle Cells

The effects of the PDGF-C on the migration of smooth muscle cells and other cells types can be assayed using the method of Koyama et al., J. Biol. Chem., 1992 267 22806-22812.

IX. Chemotaxis

The effects of the PDGF-C on chemotaxis of fibroblast, monocytes, granulocytes and other cells can be assayed using the method of Siegbahn et al., J. Clin. Invest., 1990 85 916-920.

X. PDGF-C in Other Cell Types

The effects of PDGF-C on proliferation, differentiation and function of other cell types, such as liver cells, cardiac muscle and other cells, endocrine cells and osteoblasts can readily be assayed by methods known in the art, such as ³H-thymidine uptake by in vitro cultures. Expression of PDGF-C in these and other tissues can be measured by techniques such as Northern blotting and hybridization or by in situ hybridization.

XI. Construction of PDGF-C Variants and Analogues

PDGF-C is a member of the PDGF family of growth factors which exhibits a high degree of homology to the other members of the PDGF family. PDGF-C contains eight conserved cysteine residues which are characteristic of this family of growth factors. These conserved cysteine residues form intra-chain disulfide bonds which produce the cysteine knot structure, and inter-chain disulfide bonds that form the protein dimers which are characteristic of members of the PDGF family of growth factors. PDGF-C interacts with a protein tyrosine kinase growth factor receptor.

In contrast to proteins where little or nothing is known about the protein structure and active sites needed for receptor binding and consequent activity, the design of active mutants of PDGF-C is greatly facilitated by the fact that a great deal is known about the active sites and important amino acids of the members of the PDGF family of growth factors.

Published articles elucidating the structure/activity relationships of members of the PDGF family of growth factors include for PDGF: Oestman et al., J. Biol. Chem., 1991 266 10073-10077; Andersson et al., J. Biol. Chem., 1992 267 11260-1266; Oefner et al., EMBO J., 1992 11 3921-3926; Flemming et al., Molecular and Cell Biol., 1993 13 4066-4076 and Andersson et al., Growth Factors, 1995 12 159-164; and for VEGF: Kim et al., Growth Factors, 1992 7 53-64; Pötgens et al., J. Biol. Chem., 1994 269 32879-32885 and Claffey et al., Biochem. Biophys. Acta, 1995 1246 1-9. From these publications it is apparent that because of the eight conserved cysteine residues, the members of the PDGF family of growth factors exhibit a characteristic knotted folding structure and dimerization, which result in formation of three exposed loop regions at each end of the dimerized molecule, at which the active receptor binding sites can be expected to be located.

Based on this information, a person skilled in the biotechnology arts can design PDGF-C mutants with a very high probability of retaining PDGF-C activity by conserving the eight cysteine residues responsible for the knotted folding arrangement and for dimerization, and also by conserving, or making only conservative amino acid substitutions in the likely receptor sequences in the loop 1, loop 2 and loop 3 region of the protein structure.

The formation of desired mutations at specifically targeted sites in a protein structure is considered to be a standard technique in the arsenal of the protein chemist (Kunkel et al., Methods in Enzymol., 1987 154 367-382). Examples of such site-directed mutagenesis with VEGF can be found in Pötgens et al., J. Biol. Chem., 1994 269 32879-32885 and Claffey et al., Biochem. Biophys. Acta, 1995 1246 1-9. Indeed, site-directed mutagenesis is so common that kits are commercially available to facilitate such procedures (e.g. Promega 1994-1995 Catalog., Pages 142-145).

The connective tissue cell, fibroblast, myofibroblast and glial cell growth and/or motility activity, the endothelial cell proliferation activity, the angiogenesis activity and/or the wound healing activity of PDGF-C mutants can be readily confirmed by well established screening procedures. For example, a procedure analogous to the endothelial cell mitotic assay described by Claffey et al., (Biochem. Biophys. Acta., 1995 1246 1-9) can be used. Similarly the effects of PDGF-C on proliferation of other cell types, on cellular differentiation and on human metastasis can be tested using methods which are well known in the art.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof.

What is claimed is:

1. An isolated nucleic acid molecule comprising a polynucleotide sequence having at least 85% identity with SEQ ID NO:2, SEQ ID NO:4 or SEQ ID NO:6

2. An isolated nucleic acid molecule according to claim 1, wherein the sequence identity is at least 90%.

3. An isolated nucleic acid molecule according to claim 1, wherein the sequence identity is at least 95%.

4. An isolated nucleic acid molecule according to claim 1, wherein said nucleic acid is a cDNA.

5. An isolated nucleic acid molecule according to claim 1, wherein said nucleic acid is a mammalian polynucleotide.

6. An isolated nucleic acid molecule according to claim 5, wherein said nucleic acid is a murine polynucleotide.

7. An isolated nucleic acid molecule according to claim 6, comprising SEQ ID NO:6.

8. An isolated nucleic acid molecule according to claim 5, wherein said nucleic acid is a human polynucleotide.

9. An isolated nucleic acid molecule according to claim 8, comprising SEQ ID NO:2 or SEQ ID NO:4.

10. An isolated nucleic acid molecule which encodes a polypeptide molecule comprising the amino acid sequence
PXCXXVXRCGGXXXCC (SEQ ID NO:1)
and having at least 85% identity with SEQ ID NO:3 or SEQ ID NO:5, or a fragment or analog thereof having the biological activity of PDGF-C.

11. An isolated nucleic acid molecule according to claim 10, wherein the amino acid sequence identity is at least 90%.

12. An isolated nucleic acid molecule according to claim 10, wherein the amino acid sequence identity is at least 95%.

13. An isolated nucleic acid molecule according to claim 10, which codes for a polypeptide which comprises a proteolytic site having the amino acid sequence RKSR or a structurally conserved amino acid sequence thereof.

14. A vector comprising a nucleic acid according to claim 1, which nucleic acid is operably linked with a promoter sequence.

15. A vector according to claim 14, wherein said vector is a eukaryotic vector.

16. A vector according to claim 14, wherein said vector is a prokaryotic vector.

17. A vector according to claim 14, wherein said vector is a plasmid.

18. A vector according to claim 14, wherein said vector is a baculovirus vector.

19. A method of making a vector which expresses a polypeptide comprising an amino acid sequence having at least 85% identity with SEQ ID NO:3 or SEQ ID NO:7, or fragment or analog thereof having the biological activity of PDGF-C, said method comprising incorporating an isolated nucleic acid according to claim 1 into said vector in operatively linked relation with a promoter.

20. A host cell transformed or transfected with a vector according to claim 14.

21. A host cell according to claim 20, wherein said host cell is a eukaryotic cell.

22. A host cell according to claim 20, wherein said host cell is a COS cell.

23. A host cell according to claim 20, wherein said host cell is a prokaryotic cell.

24. A host cell according to claim 20, wherein said host cell is a 293EBNA cell.

25. A host cell according to claim 20, wherein said host cell is an insect cell.

26. A host cell transformed or transfected with a vector comprising a nucleic acid sequence according to claim 1, operatively linked to a promoter, such that said host cell expresses a polypeptide comprising an amino acid sequence having at least 85% identity with SEQ ID NO:3 or SEQ ID NO:7, or a fragment or analog thereof having the biological activity of PDGF-C.

27. A means for amplifying a polynucleotide according to claim 1 in a test sample, said means comprising at least one pair of primers complementary to a nucleic acid according to claim 1.

28. A means for amplifying a polynucleotide according to claim 1 in a test sample, said means comprising a polymerase and at least one pair of primers complementary to a nucleic acid according to claim 1, for amplifying the polynucleotide by polymerase chain reaction in order to facilitate a sequence comparison of the polynucleotide with the nucleic acid according to claim 1.

29. An antibody specifically reactive with a polypeptide comprising an amino acid sequence having at least 85% identity with SEQ ID NO:3 or SEQ ID NO:7, or a fragment or analog thereof having the biological activity of PDGF-C, or a polypeptide produced by expression of a polynucleotide comprising a polynucleotide sequence having at least 85% identity with SEQ ID NO:2, SEQ ID NO:4 or SEQ ID NO:6, or of a polynucleotide which hybridizes under stringent conditions with SEQ ID NO:2, SEQ ID NO:4 or SEQ ID NO:6.

30. An antibody according to claim 29, wherein said antibody is a polyclonal antibody.

31. An antibody according to claim 29, wherein said antibody is a monoclonal antibody or a F(ab′)₂, F(ab′), F(ab) fragment or chimeric antibody.

32. An antibody according to claim 29, wherein said antibody is labeled with a detectable label.

33. An antibody according to claim 32, wherein said detectable label is radioactive isotope.

34. An antibody according to claim 31, wherein said monoclonal antibody is a humanized antibody.

35. A method of making a polypeptide comprising an amino acid sequence having at least 85% identity with SEQ ID NO:3 or SEQ ID NO:7, or a fragment or analog thereof having the biological activity of PDGF-C, or a polypeptide produced by expression of a polynucleotide comprising a polynucleotide sequence having at least 85% identity with SEQ ID NO:2, SEQ ID NO:4 or SEQ ID NO:6, or of a polynucleotide which hybridizes under stringent conditions with SEQ ID NO:2, SEQ ID NO:4 or SEQ ID NO:6, said method comprising the steps of:
culturing a host cell transformed or transfected with a vector comprising a polynucleotide encoding said polypeptide operably associated with a promoter sequence such that the nucleic acid sequence encoding said polypeptide is expressed; and
isolating said polypeptide from said host cell or from a growth medium in which said host cell is cultured.

36. A method of stimulating growth of connective tissue or wound healing in a mammal, said method comprising administering to said mammal an effective growth stimulating amount of a polypeptide comprising an amino acid sequence having at least 85% identity with SEQ ID NO:3 or SEQ ID NO:7, or a fragment or analog thereof having the biological activity of PDGF-C, or a polypeptide produced by expression of a polynucleotide comprising a polynucleotide sequence having at least 85% identity with SEQ ID NO:2, SEQ ID NO:4 or SEQ ID NO:6, or of a polynucleotide which hybridizes under stringent conditions with SEQ ID NO:2, SEQ ID NO:4 or SEQ ID NO:6.

37. A method of making a vector which expresses a polypeptide comprising an amino acid sequence having at least .85% identity with at least amino acid residues 230 to 345 of SEQ ID NO:3 or of SEQ ID NO:7, said method comprising incorporating an isolated nucleic acid molecule encoding said amino acid residues into said vector in operatively linked relation with a promoter.

38. A method for producing an active truncated form of PDGF-C, comprising the step of expressing an expression vector comprising a polypeptide-encoding polynucleotide as claimed in claim 37.

39. A method for regulating receptor-binding specificity of PDGF-C, comprising the steps of expressing an expression vector comprising a polynucleotide encoding a polypeptide comprising an amino acid sequence having at least 85% identity with SEQ ID NO:3 or SEQ ID NO:7, or a fragment or analog thereof having the biological activity of PDGF-C, or a polypeptide produced by expression of a polynucleotide comprising a polynucleotide sequence having at least 85% identity with SEQ ID NO:2, SEQ ID NO:4 or SEQ ID NO:6, or of a polynucleotide which hybridizes under stringent conditions with SEQ ID NO:2, SEQ ID NO:4 or SEQ ID NO:6, and supplying a proteolytic amount of at least one enzyme for processing the expressed polypeptide to generate the active truncated form of PDGF-C.

40. A method for selectively activating a polypeptide having a growth factor activity comprising the step expressing an expression vector comprising a polynucleotide encoding a polypeptide having a growth factor activity, a CUB domain and a proteolytic site between the polypeptide and the CUB domain, and supplying a proteolytic amount of at least one enzyme for processing the expressed polypeptide to generate the active polypeptide having a growth factor activity.

41. An isolated heterodimer comprising an active monomer of VEGF, VEGF-B, VEGF-C, VEGF-D, PDGF-C, PDGF-A, PDGF-B or PlGF and an active monomer of PDGF-C linked to a CUB domain.

42. An isolated heterodimer according to claim 41, further comprising a proteolytic site between the active monomer and the CUB domain linkage.

43. An isolated heterodimer comprising an active monomer of PDGF-C and an activated monomer of VEGF, VEGF-B, VEGF-C, VEGF-D, PDGF-C, PDGF-A, PDGF-B or PlGF linked to a CUB domain.

44. An isolated heterodimer according to claim 43, further comprising a proteolytic site between the activated monomer and the CUB domain linkage.

45. An isolated polynucleotide, comprising a polynucleotide sequence having at least 85% identity with SEQ ID NO:2, SEQ ID NO:4 or SEQ ID NO:6, or a polynucleotide which hybridizes under stringent conditions with SEQ ID NO:2, SEQ ID NO:4 or SEQ ID NO:6, and which encodes a sequence of amino acids comprising SEQ ID NO:1.

46. A method of promoting fibroblast mitogenesis in a mammal, comprising the step of administering to said mammal an effective fibroblast mitogenesis promoting amount of a polypeptide comprising an amino acid sequence having at least 85% identity with at least amino acid residues 230 to 345 of SEQ ID NO:3 or of SEQ ID NO:7.

47. A method of promoting fibroblast mitogenesis in a mammal, comprising administering to said mammal an effective frbroblast mitogenesis promoting amount of a polypeptide comprising an amino acid sequence having at least 85% identity with SEQ ID NO:3 or SEQ ID NO:7, or a fragment or analog thereof having the biological activity of PDGF-C, or a polypeptide produced by expression of a polynucleotide comprising a polynucleotide sequence having at least 85% identity with SEQ ID NO:2, SEQ ID NO:4 or SEQ ID NO:6, or of a polynucleotide which hybridizes under stringent conditions with SEQ ID NO:2, SEQ ID NO:4 or SEQ ID NO:6.

48. A method of inducing PDGF alpah receptor activation, comprising the step of adding a PDGF alpha-receptor stimulating amount of a polypeptide comprising an amino acid sequence having at least 85% identity with at least amino acid residues 230 to 345 of SEQ ID NO:3 or of SEQ ID NO:7.

49. A method of inducing PDGF alpha receptor activation, comprising the step of adding a PDGF alpha-receptor stimulating amount of a polypeptide comprising an amino acid sequence having at least 85% identity with SEQ ID NO:3 or SEQ ID NO:7, or a fragment or analog thereof having the biological activity of PDGF-C, or a polypeptide produced by expression of a polynucleotide comprising a polynucleotide sequence having at least 85% identity with SEQ ID NO:2, SEQ ID NO:4 or SEQ ID NO:6, or of a polynucleotide which hybridizes under stringent conditions with SEQ ID NO:2, SEQ ID NO:4 or SEQ ID NO:6.

50. A method of inhibiting tumor growth of a tumor expressing PDGF-C in a mammal, comprising administering to said mammal a PDGF-C inhibiting amount of a PDGF-C antagonist.

51. A method of identifying specific types of human tumors, comprising the step of taking a sample of the tumor and testing for the expression of PDGF-C.

52. The method of claim 51, wherein the specific types of tumors are selected from the group consisting of choriocarcinoma, Wilms tumor, megakaryoblastic leukemia, lung carcinoma and erythroleukemia.

53. A method for identifying an PDGF-C antagonist comprising:
admixing a substantially purified preparation of an activated truncated form of PDGF-C; and
monitoring, by any suitable means, an inhibition in the biological activity of PDGF-C.

54. A method for identifying an PDGF-C antagonist comprising:
admixing a substantially purified preparation of an full-length PDGF-C with a test agent; and
monitoring, by any suitable means, an inhibition in the cleavage of the CUB domain from PDGF-C.

55. A method for producing an activated truncated form of PDGF-C, comprising the steps of:
expressing an expression vector comprising a polynucleotide encoding a polypeptide comprising an amino acid sequence having at least 85% identity with SEQ ID NO:3 or SEQ ID NO:7, or a fragment or analog thereof having the biological activity of PDGF-C, or comprising a polynucleotide comprising a polynucleotide sequence having at least 85% identity with SEQ ID NO:2, SEQ ID NO:4 or SEQ ID NO:6, or a polynucleotide which hybridizes under stringent conditions with SEQ ID NO:2, SEQ ID NO:4 or SEQ ID NO:6, and
supplying a proteolytic amount of at least one enzyme for processing the expressed polypeptide to generate the activated truncated form of PDGF-C.

56. A method of inhibiting tissue remodeling during invasion of tumor cells into a normal population of cells, comprising administering to said mammal a PDGF-C inhibiting amount of a PDGF-C antagonist.

57. A method of treating fibrotic conditions in a mammal in need a such treatment, comprising administering to said mammal a PDGF-C inhibiting amount of a PDGF-C antagonist.

58. A method of claim 57, wherein the fibrotic conditions are found in the lung, kidney or liver.

59. A method of promoting angiogenesis in a bird or mammal, said method comprising administering to said bird or mammal an effective angiogenesis promoting amount of a polypeptide comprising a sequence of amino acids having at least 85% identity with at least amino acid residues 230 to 345 of SEQ ID NO:3 or of SEQ ID NO:7.

60. A method according to claim 59, wherein said polypeptide is administered in the form of a dimer.


SEQUENCE LISTING (39 sequences)

SEQ ID NO: 1 - protein, 16 aa, Homo sapiens
  UNSURE (2): Can be any amino acid residue
PXCLLVXRCGGXCXCC

SEQ ID NO: 2 - DNA, 2108 bp, Homo sapiens
  unsure (2002): can be a, c, g or t
ccccgccgtgagtgagctctcaccccagtcagccaaatgagcctcttcgggcttctcctggtgacatctgccctggccggccagagacgagggactcaggcggaatccaacctgagtagtaaattccagttttccagcaacaaggaacagaacggagtacaagatcctcagcatgagagaattattactgtgtctactaatggaagtattcacagcccaaggtttcctcatacttatccaagaaatacggtcttggtatggagattagtagcagtagaggaaaatgtatggatacaacttacgtttgatgaaagatttgggcttgaagacccagaagatgacatatgcaagtatgattttgtagaagttgaggaacccagtgatggaactatattagggcgctggtgtggttctggtactgtaccaggaaaacagatttctaaaggaaatcaaattaggataagatttgtatctgatgaatattttccttctgaaccagggttctgcatccactacaacattgtcatgccacaattcacagaagctgtgagtccttcagtgctacccccttcagctttgccactggacctgcttaataatgctataactgcctttagtaccttggaagaccttattcgatatcttgaaccagagagatggcagttggacttagaagatctatataggccaacttggcaacttcttggcaaggcttttgtttttggaagaaaatccagagtggtggatctgaaccttctaacagaggaggtaagattatacagctgcacacctcgtaacttctcagtgtccataagggaagaactaaagagaaccgataccattttctggccaggttgtctcctggttaaacgctgtggtgggaactgtgcctgttgtctccacaattgcaatgaatgtcaatgtgtcccaagcaaagttactaaaaaataccacgaggtccttcagttgagaccaaagaccggtgtcaggggattgcacaaatcactcaccgacgtggccctggagcaccatgaggagtgtgactgtgtgtgcagagggagcacaggaggatagccgcatcaccaccagcagctcttgcccagagctgtgcagtgcagtggctgattctattagagaacgtatgcgttatctccatccttaatctcagttgtttgcttcaaggacctttcatcttcaggatttacagtgcattctgaaagaggagacatcaaacagaattaggagttgtgcaacagctcttttgagaggaggcctaaaggacaggagaaaaggtcttcaatcgtggaaagaaaattaaatgttgtattaaatagatcaccagctagtttcagagttaccatgtacgtattccactagctgggttctgtatttcagttctttcgatacggcttagggtaatgtcagtacaggaaaaaaactgtgcaagtgagcacctgattccgttgccttgcttaactctaaagctccatgtcctgggcctaaaatcgtataaaatctggatttttttttttttttttgctcatattcacatatgtaaaccagaacattctatgtactacaaacctggtttttaaaaaggaactatgttgctatgaattaaacttgtgtcrtgctgataggacagactggatttttcatatttcttattaaaatttctgccatttagaagaagagaactacattcatggtttggaagagataaacctgaaaagaagagtggccttatcttcactttatcgataagtcagtttatttgtttcattgtgtacatttttatattctccttttgacattataactgttggcttttctaatcttgttaaatatatctatttttaccaaaggtatttaatattcttttttatgacaacttagatcaactatttttagcttggtaaatttttctaaacacaattgttatagccagaggaacaaagatggatataaaaatattgttgccctggacaaaaatacatgtatntccatcccggaatggtgctagagttggattaaacctgcattttaaaaaacctgaattgggaanggaanttggtaaggttggccaaancttttttgaaaataattaa

SEQ ID NO: 3 - protein, 345 aa, Homo sapiens [sequence omitted]

SEQ ID NO: 4 - DNA, 1536 bp, Homo sapiens
cgggtaaattccagttttccagcaacaaggaacagaacggagtacaagatcctcagcatgagagaattattactgtgtctactaatggaagtattcacagcccaaggtttcctcatacttatccaagaaatacggtcttggtatggagattagtagcagtagaggaaaatgtatggatacaacttacgtttgatgaaagatttgggcttgaagacccagaagatgacatatgcaagtatgattttgtagaagttgaggaacccagtgatggaactatattagggcgctggtgtggttctggtactgtaccaggaaaacagatttctaaaggaaatcaaattaggataagatttgtatctgatgaatattttccttctgaaccagggttctgcatccactacaacattgtcatgccacaattcacagaagctgtgagtccttcagtgctacccccttcagctttgccactggacctgcttaataatgctataactgcctttagtaccttggaagaccttattcgatatcttgaaccagagagatggcagttggacttagaagatctatataggccaacttggcaacttcttggcaaggcttttgtttttggaagaaaatccagagtggtggatctgaaccttctaacagaggaggtaagattatacagctgcacacctcgtaacttctcagtgtccataagggaagaactaaagagaaccgataccattttctggccaggttgtctcctggttaaacgctgtggtgggaactgtgcctgttgtctccacaattgcaatgaatgtcaatgtgtcccaagcaaagttactaaaaaataccacgaggtccttcagttgagaccaaasaccggtgtcaggggattgcacaaatcactcaccgacgtggccctggagcaccatgaggagtgtgactgtgtgtgcagagggagcacaggaggatagccgcatcaccaccagcagctcttgcccagagctgtgcagtgcagtggctgattctattagagaacgtatgcgttatctccatccttaatctcagttgtttgcttcaaggacctttcatcttcaggatttacagtgcattctgaaagaggagacatcaaacagaattaggagttgtgcaacagctcttttgagaggaggcctaaaggacaggagaaaaggtcttcaatcgtggaaagaaaattaaatgttgtattaaatagatcaccagctagtttcagagttaccatgtacgtattccactagctgggttctgtatttcagttctttcgatacggcttagggtaatgtcagtacaggaaaaaaactgtgcaagtgagcacctgattccgttgccttgcttaactctaaagctccatgtcctgggcctaaaatcgtataaaatctggatttttttttttttttttgctcatattcacatatgtaaaccagaacattctatgtactacaaacctggtttttaaaaaggaactatgttgctatgaattaaacttgtgtcatgctgataggacagactgga

SEQ ID NO: 5 - protein, 318 aa, Homo sapiens
GKFQFSSNKEQNGVQDPQHERIITVSTNGSIHSPRFPHTYPRNTVLVWRLVAVEENVWIQLTFDERFGLEDPEDDICKYDFVEVEEPSDGTILGRWCGSGTVPGKQISKGNQIRIRFVSDEYFPSEPGFCIHYNIVMPQFTEAVSPSVLPPSALPLDLLNNAITAFSTLEDLIRYLEPERWQLDLEDLYRPTWQLLGKAFVFGRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

SEQ ID NO: 6 - DNA, 1474 bp, Murinae gen. sp.
  unsure (1447): can be a, c, g or t
cacctggagacacagaagagggctctaggaaaaattttggatggggattatgtggaaactaccctgcgattctctgctgccagagccggccaggcgcttccaccgcagcgcagcctttccccgggctgggctgagccttggagtcgtcgcttccccagtgcccgccgcgagtgagccctcgccccagtcagccaaatgctcctcctcggcctcctcctgctgacatctgccctggccggccaaagaacggggactcgggctgagtccaacctgagcagcaagttgcagctctccagcgacaaggaacagaacggagtgcaagatccccggcatgagagagttgtcactatatctggtaatgggagcatccacagcccgaagtttcctcatacgtacccaagaaatatggtgctggtgtggagattagttgcagtagatgaaaatgtgcggatccagctgacatttgatgagagatttgggctggaagatccagaagacgatatatgcaagtatgattttgtagaagttgaggagcccagtgatggaagtgttttaggacgctggtgtggttctgggactgtgccaggaaagcagacttctaaaggaaatcatatcaggataagatttgtatctgatgagtattttccatctgaacccggattctgcatccactacagtattatcatgccacaagtcacagaaaccacgagtccttcggtgttgcccccttcatctttgtcattggacctgctcaacaatgctgtgactgccttcagtaccttggaagagctgattcggtacctagagccagatcgatggcaggtggacttggacagcctctacaagccaacatggcagcttttgggcaaggctttcctgtatgggaaaaaaagcaaagtggtgaatctgaatctcctcaaggaagaggtaaaactctacagctgcacaccccggaacttctcagtgtccatacgggaagagctaaagaggacagataccatattctggccaggttgtctcctggtcaagcgctgtggaggaaattgtgcctgttgtctccataattgcaatgaatgtcagtgtgtcccacgtaaagttacaaaaaagtaccatgaggtccttcagttgagaccaaaaactggagtcaagggattgcataagtcactcactgatgtggctctggaacaccacgaggaatgtgactgtgtgtgtagaggaaacgcaggagggtaactgcagccttcgtagcagcacacgtgagcactggcattctgtgtacccccacaagcaaccttcatccccaccagcgttggccgcagggctctcagctgctgatgctggctatggtaaagatcttactcgtctccaaccaaattctcagttgtttgcttcaatagccttcccctgcaggacttcaagtgtcttctaaaagaccagaggcaccaanaggagtcaatcacaaagcactgcaccg

SEQ ID NO: 7 - protein, 345 aa, Murinae gen. sp.
MLLLGLLLLTSALAGQRTGTRAESNLSSKLQLSSDKEQNGVQDPRHERVVTISGNGSIHSPKFPHTYPRNMVLVWRLVAVDENVRIQLTFDERFGLEDPEDDICKYDFVEVEEPSDGSVLGRWCGSGTVPGKQTSKGNHIRIRFVSDEYFPSEPGFCIHYSIIMPQVTETTSPSVLPPSSLSLDLLNNAVTAFSTLEELIRYLEPDRWQVDLDSLYKPTWQLLGKAFLYGKKSKVVNLNLLKEEVKLYSCTPRNFSVSIREELKRTDTIFWPGCLLVKRCGGNCACCLHNCNECQCVPRKVTKKYHEVLQLRPKTGVKGLHKSLTDVALEHHEECDCVCRGNAGG

SEQ ID NO: 8 - protein, 192 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSSERRKHLFVQDPQTCKCSCKNTDSRCKARQLELNERTCRCDKPRR

SEQ ID NO: 9 - protein, 170 aa, Homo sapiens [sequence omitted]

SEQ ID NO: 10 - protein, 188 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDSPRPLCPRCTQHHQRPDPRTCRCRCRRRSFLRCQGRGLELNPDTCRCRKLRR

SEQ ID NO: 11 - protein, 133 aa, Homo sapiens
MKLLVGILVAVCLHQYLLNADSNTKGWSEVLKGSECKPRPIVVPVSETHPELTSQRFNPPCVTLMRCGGCCNDESLECVPTEEVNVSMELLGASGSGSNGMQRLSFVEHKKCDCRPRFTTTPPTTTRPPRRRR

SEQ ID NO: 12 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 13 - protein, 358 aa, Homo sapiens
MYGEWGMGNILMMFHVYLVQGFRSEHGPVKDFSFERSSRSMLERSEQQIRAASSLEELLQIAHSEDWKLWRCRLKLKSLASMDSRSASHRSTRFAATFYDTETLKVIDEEWQRTQCSPRETCVEVASELGKTTNTFFKPPCVNVFRCGGCCNEEGVMCMNTSTSYISKQLFEISVPLTSVPELVPVKIANHTGCKCLPTGPRHPYSIIRRSIQTPEEDECPHSKKLCPIDMLWDNTKCKCVLQDETPLPGTEDHSYLQEPTLCGPHMTFDEDRCECVCKAPCPGDLIQHPENCSCFECKESLESCCQKHKIFHPDTCSCEDRCPFHTRTCASRKPACGKHWRFPKETRAQGLYSQENP

SEQ ID NO: 14 - protein, 211 aa, Homo sapiens [sequence omitted]

SEQ ID NO: 15 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 16 - protein, 182 aa, Homo sapiens
MPQFTDCVCRGSTGGEAVSPSVLPPSALPLDLLNNAITAFSTLEDLIRYLEPERWQLDLEDLYRPTWQLLGKAFVFGRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEEC

SEQ ID NO: 17 - protein, 182 aa, Murinae gen. sp.
MPQVTETTSPSVLPPSSLSLDLLNNAVTAFSTLEELIRYLEPDRWQVDLDSLYKPTWQLDCVCRGNAGGLGKAFLYGKKSKVVNLNLLKEEVKLYSCTPRNFSVSIREELKRTDTIFWPGCLLVKRCGGNCACCLHNCNECQCVPRKVTKKYHEVLQLRPKTGVKGLHKSLTDVALEHHEEC

SEQ ID NO: 18 - protein, 117 aa, Murinae gen. sp.
ERVVTISGNGSIHSPKFPHTYPRNMVLVWRLVAVDENVRIQLTFDERFGLEDPEDDICKYDFVEVEEPSDGSVLGRWCGSGTVPGKQTSKGNMIRIRFVSDEYFPSEPGFCIHYSII

SEQ ID NO: 19 - protein, 117 aa, Homo sapiens
ERIITVSTNGSIHSPRFPHTYPRNTVLVWRLVAVEENVWIQLTFDERFGLEDPEDDICKYDFVEVEEPSDGTILGRWCGSGTVPGKQISKGNQIRIRFVSDEYFPSEPGFCIHYNIV

SEQ ID NO: 20 - protein, 113 aa, Homo sapiens
CGETLQDSTGNFSSPEYPNGYSAHMHCVWRISVTPGEKIILNFTSLDLYRSRLCWYDYVEVRDGFWRKAPLRGRFCGSKLPEPIVSTDSRLWVEFRSSSNWVGKGFFAVYEAI

SEQ ID NO: 21 - protein, 112 aa, Homo sapiens
CGGDVKKDYGHIQSPNYPDDYRPSKVCIWRIQVSEGFHVGLTFQSFEIERMDSCAYDYLEVRDGHSESSTLIGRYCGYEKPDDIKSTSSRLWLKFVSDGSINKAGFAVNFFK

SEQ ID NO: 22 - protein, 113 aa, Homo sapiens
CGGFLTKLNGSITSPGWPKEYPPNKNCIWQLVAPTQYRISLQFDFFETEGNDVCKYDFVEVRSGLTADSKLHGKFCGSEKPEVITSQYNNMRVEPKSDNTVSKKGFKAHFFSE

SEQ ID NO: 23 - protein, 113 aa, Homo sapiens
GDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEI

SEQ ID NO: 24 - protein, 119 aa, Homo sapiens
CSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVL

SEQ ID NO: 25 - DNA, 19 bp, Homo sapiens
gaagttgaggaacccagtg

SEQ ID NO: 26 - DNA, 20 bp, Homo sapiens
cttgccaagaagttgccaag

SEQ ID NO: 27 - DNA, 19 bp, Murinae gen. sp.
cttcagtaccttggaagag

SEQ ID NO: 28 - DNA, 19 bp, Murinae gen. sp.
cgcttgaccaggagacaac

SEQ ID NO: 29 - DNA, 30 bp, Murinae gen. sp.
acgtgaattcagcaagttcagcctggttaa

SEQ ID NO: 30 - DNA, 30 bp, Murinae gen. sp.
acgtggatcctgagtatttcttccagggta

SEQ ID NO: 31 - protein, 22 aa, Homo sapiens
CKFQFSSNKEQNGVQDPQHERC

SEQ ID NO: 32 - protein, 21 aa, Homo sapiens
GRKSRVVDLNLLTEEVRLYSC

SEQ ID NO: 33 - DNA, 26 bp, Homo sapiens
cgggatcccgaatccaacctgagtag

SEQ ID NO: 34 - DNA, 61 bp, Homo sapiens
ggaattcctaatggtgatggtgatgatgtttgtcatcgtcatctcctcctgtgctccctct

SEQ ID NO: 35 - DNA, 29 bp, Homo sapiens
cggatcccggaagaaaatccagagtggtg

SEQ ID NO: 36 - DNA, 61 bp, Homo sapiens; identical to SEQ ID NO: 34

SEQ ID NO: 37 - protein, 21 aa, Homo sapiens
GRKSRVVDLNLLTEEVRLYSC

SEQ ID NO: 38 - DNA, 26 bp, Homo sapiens
  Forward PCR primer from the human PDGF-C 430 bp cDNA fragment encoding the CUB domain which includes a BamHI site
cgggatcccgaatccaacctgagtag

SEQ ID NO: 39 - DNA, 60 bp, Homo sapiens
  Reverse  PCR primer from the human PDGF-C 430 bpcDNA fragment encoding the CUB domain which includes a EcoRI site and sequences coding for a C-terminal 6X His tag preceded by an enterokinase site
ccggaattcctaatggtgatggtgatgatgtttgtcatcgtcgtcgacaatgttgtagtg